(19) United States
(12) Patent Application Publication
(10) Pub. No.: US 2025/0320526 A1
(43) Pub. Date: Oct. 16, 2025

(54) METHOD FOR PRODUCTION OF A EUKARYOTIC HOST CELL OR CELL LINE FOR LAMBDA-INTEGRASE-MEDIATED RECOMBINATION

(71) Applicants: NANYANG TECHNOLOGICAL UNIVERSITY, Singapore (SG); LAMBDAGEN PTE. LTD., Havelock 2 (SG)

(72) Inventors: Asim Azhar SIDDIQUI, Singapore (SG); Harshyaa MAKHIJA, Havelock 2 (SG); Peter DRÖGE, Singapore (SG)

(21) Appl. No.: 18/865,903

(22) PCT Filed: May 31, 2023
(86) PCT No.: PCT/SG2023/050387

§ 371 Date: Nov. 14, 2024

(30) Foreign Application Priority Data
May 31, 2022 (SG) 10202250058R

(51) Int. Cl. C12N 15/90; C12N 15/85

(52) U.S. Cl. CPC C12N 15/907; C12N 15/85

(57) ABSTRACT

The present invention relates generally to the field of site-specific DNA recombination mediated by lambda integrases, and more specifically to methods of producing eukaryotic cells and cell lines comprising a genomic landing pad for lambda integrase mediated recombination, as well as the eukaryotic cells themselves and subsequent methods of their use for lambda integrase mediated recombination and as bioreactors for cell therapies.

### CROSS-REFERENCE TO RELATED APPLICATION

This application claims the benefit of priority of Singapore Patent Application No. 10202250058R filed 31 May 2022, the content of which being hereby incorporated by reference in its entirety for all purposes.

### FIELD OF THE INVENTION

The present invention relates generally to the field of site-specific DNA recombination mediated by lambda integrases, and more specifically to methods of producing eukaryotic cells and cell lines comprising a genomic landing pad for lambda integrase mediated recombination, as well as the eukaryotic cells themselves and subsequent methods of their use for lambda integrase mediated recombination and as bioreactors for cell therapies.

### BACKGROUND OF THE INVENTION

Therapeutic products that are derived from living organisms are known as biotherapeutics and are the fastest-growing categories of products in the pharmaceutical industry including, but not limited to, monoclonal antibodies, signaling molecules and blood factors that are being produced in mammalian cell lines. Hence, the development and manufacturing of biotherapeutics hinge on genetically stable producer and/or tester cells capable of producing recombinant proteins efficiently. Furthermore, emerging cell encapsulation technologies have enabled possible new applications for mammalian producer cells as mini-bioreactors for in vivo cell-based therapies.

Mammalian cells have certain advantages over other expression systems such as those derived from bacterial, yeast or insect origin. They have the desired features to express large and complex proteins with proper folding and post translational modifications. Chinese Hamster Ovary (CHO) cells, an immortalized epithelial cell line, are the current workhorse of the biopharmaceutical industry resistant to human pathogen infection. Although CHO cells are used for cost-effective mass production of therapeutic proteins, some shortcomings do exist. The protein glycosylation pattern of CHO cells is different from that of human cells and there is a significant risk of genetic instability [1]. Furthermore, CHO cells produce their own glycans, such as α-gal and N-glycolylneuraminic acid, which are absent from human cells. As a result, recombinant proteins may fail to function or may trigger immune responses [2]. In addition, transgene silencing and productivity loss have been attributed to DNA methylation and histone modifications in CHO cells [3,4]. Therefore, alternative cell-based systems are being sought including those of human origin. In this context, Expi293F cells are derived from immortalized human embryonic kidney cells (HEK293), hence offering human-specific post-translational modifications. These cells can grow in suspension cultures at high density to produce high levels of proteins from episomal or chromosomal transgenes [5]. HEK293 cells have a significant history of use in the development of cell and gene therapy products[6], and GMP-qualified HEK293/Expi293F cells are available[7].

Expression from episomal transgenes appears to be fast and simple and can result in high yields of biotherapeutics. However, this method exhibits batch-to-batch variation, yield depreciation with time, and high costs due to repeated gene transfers and the need for selection pressure [8]. Furthermore, the functional efficacy of variants of certain biologics is difficult to study with episomal transgenes due, e.g., to variations in transfection efficiencies.

An alternative to overcome these limitations are expression systems based on stable, genome-integrated transgenes [9]. Transgene integration can occur either randomly or sequence-specifically. Randomly integrated transgenes require screening of a large number of clones for the most efficient producer cell lines, which is a costly and time-consuming process, particularly under GMP conditions. Additionally, it is difficult to predict consistent transgene expression and the number and stability of the integrated transgenes [10]. To overcome these shortcomings, site-specific transgene integration approaches have been developed. Various well-characterized genome engineering tools and tested genomic harbour sites, that are either endogenous or artificially introduced, are being used as so-called landing pads for transgenes.

Multiple genome-editing tools like zinc finger nucleases (ZFNs), clustered regularly interspaced short palindromic repeats associated protein RNA guided nucleases (e.g. CRISPR-Cas9 system) and transcription-activator like effector nucleases (TALENs), are being used for site-specific transgene insertion[11-13]. These programmable endonucleases introduce DNA double-strand breaks at a selected locus in the genome, and during the process of repairing this break, the cellular machinery may insert the transgene expression cassette at the break site by employing homologous recombination pathways. Therefore, both the exogenous endonuclease and the cellular repair mechanism are critical to the efficiency of this method [14].

Recombinase-Mediated Cassette Exchange (RMCE) using site-specific recombinase systems such as Cre-lox, Flp-FRT, Bxb1-attP/B and ϕDC31-attP/B have also been used as genome engineering toolsClick or tap here to enter text. These enzymes can perform precise DNA recombination reactions at their respective cognate sites without a need for host factors and can lead to DNA segment insertions, deletions, or inversions [15]. In RMCE, two different recombinases (e.g. Cre and Flp) are often employed to insert the transgene construct into an artificial genomic landing pad that carries the respective pair of recombination target sequences. The landing pad locus in the host cell chromatin should be accessible for both the recombinases and incoming transgenes. In addition, it must be genetically stable for sustained, high expression of transgenes.

A number of these functional hotspots have been identified in CHO and in human cells [16]. In particular, a critical step in the generation of a master producer or tester cell line is the selection of the genomic locus where the artificial docking site (landing pad) should be inserted. Landing pads usually contain selection markers and recognition sites for site-specific DNA recombinases, which enable the precise insertion of transgene expression cassettes with minimal off-target events. The selection of the “best” genomic landing site can be achieved by computational and experimental strategies, or a combination thereof, and a number of functional hot spots in the CHO cell genome have been identified in this manner [16]. These hot spots are selected primarily based on genetic stability and sustained, high-yield transgene expression.

Recently, another editing tool based on A-phage integrase has been engineered for human genome manipulation especially for large transgene insertion reactions. The integrase was genetically modified by directed evolution to generate an enhanced, so-called IntC3 variant for mammalian cells [17], that works efficiently in the targeting of a novel endogenous human target sequence [18].

Most genome engineering approaches that are aimed to increase production of biotherapeutics have been applied to CHO cells. However, while the above-mentioned strategies and tools for CHO master cell engineering have been developed over thirty years, there is still a void when it comes to the generation of human master cell lines which can be the preferred choices for a number of biopharmaceutical and biomedical applications, e.g. cell therapies with encapsulated mini bioreactors.

Therefore, there is still need in the art for methods of producing eukaryotic cells and cell lines comprising a genomic landing pad for lambda integrase mediated recombination to address the drawbacks of existing approaches. In particular, there is a need in the art for the production of human master cell lines for transgene insertion and subsequent use in a number of biopharmaceutical and biomedical applications.

### SUMMARY OF THE INVENTION

In one aspect, the present application relates to a method for production of a eukaryotic host cell comprising a landing pad for A-integrase-mediated recombination, the method comprising:
(i) transfecting a bacterial plasmid into a eukaryotic host cell at suitable conditions to induce said transfection, wherein the bacterial plasmid comprises a landing pad comprising:
   a. a RNA polymerase promoter, more preferably EF-1α promoter;
   b. a modified lambda integrase recombination sequence attP or derivative thereof, downstream of the promoter, wherein the modified phage lambda integrase recombination sequence has been modified to remove ATG start codons; and
   c. a first fluorescent marker gene, preferably mCherry, downstream of the modified lambda integrase recombination sequence and operably linked to the RNA polymerase promoter;
(ii) culturing the transfected eukaryotic host cell under conditions that allow expression of the first fluorescent marker gene;
(iii) identifying a eukaryotic host cell into which the bacterial plasmid has stably integrated based on the detectable expression of the first fluorescent marker gene, preferably by flow cytometry; and
(iv) isolating the identified eukaryotic host cell to obtain the eukaryotic host cell or cell line comprising the genomic landing pad for λ-integrase-mediated recombination.

In various embodiments, the eukaryotic host cell is a higher eukaryotic host cell, preferably a mammalian host cell, more preferably a human cell, more preferably an embryonic kidney 293 (HEK 293) cell such as a human Expi293F cell.

In various embodiments, the eukaryotic host cell is a human cell, more preferably a human Expi293F cell, wherein the RNA polymerase promoter is a EF-1α promoter; wherein the modified lambda integrase recombination sequence attP comprises or consists of a nucleotide sequence according to SEQ ID NO:5 or a derivative thereof, and is downstream of the promoter; and wherein the first fluorescent marker gene is downstream of the modified lambda integrase recombination sequence attP sequence and is operably linked to the EF-1α promoter.

In various embodiments, the landing pad further comprises one or more additional recombination sequences such as loxP and/or FRT and/or attP.

In various embodiments, step (iv) comprises serial dilution or single cell FACS of the identified eukaryotic host cell to obtain the clonal eukaryotic host cell line comprising the genomic landing pad for λ-integrase-mediated recombination.

In various embodiments, the method further comprises generating a eukaryotic host cell line comprising the genomic landing pad, preferably the eukaryotic host cell line is a monoclonal cell line.

In various embodiments, the eukaryotic host cell line exhibits homogenous and stable long-term expression levels of the first fluorescent marker gene, preferably confirmed by flow cytometry analysis, in the absence of selection pressure.

In various embodiments, the method further comprises, after step (iv), screening the isolated eukaryotic host cells for competency of λ-integrase-mediated recombination.

In various embodiments, the method further comprises, after step (iv), confirming the eukaryotic host cell of step (iv) contains a single copy of landing pad by Southern blotting analysis.

In various embodiments, the method further comprises, after step (iv), confirming the integration of the landing pad into the genome of the eukaryotic host cell, by PCR analysis.

In another aspect, the invention relates to a eukaryotic host cell or cell line obtained by the methods disclosed herein.

In another aspect, the invention relates to a method of λ-integrase-mediated insertion of a DNA sequence of interest into a eukaryotic host cell, the method comprising:
(i) providing a first circular DNA molecule comprising a lambda integrase recombination partner sequence of attP, and a DNA sequence of interest comprising a selection marker gene, preferably a hygromycin resistance gene, and a second fluorescent marker gene, preferably eGFP, operably linked to a constitutive promoter;
(ii) providing a second circular DNA molecule comprising a nucleotide sequence encoding a lambda integrase (IntC3) having the amino acid sequence set forth in SEQ ID NO:6 or a functional variant or fragment thereof;
(iii) co-transfecting a landing pad eukaryotic cell disclosed herein with the first circular DNA molecule of (i) and the second circular DNA molecule of (ii) at suitable conditions to induce said co-transfection; and
(iv) inducing the expression of the lambda integrase to facilitate recombination and stable integration of the first circular DNA construct into the landing pad comprised in the eukaryotic host cell;
(v) identifying a eukaryotic host cell comprising a landing pad into which the first circular DNA construct has stably integrated based on the detectable expression of the selection marker gene and second fluorescent marker gene, and the absence of the detectable expression of the first fluorescent marker gene.

In various embodiments, step (iv) results in the creation of two genomic recombination junction sequences flanking the DNA sequence of interest.

In various embodiments, the first fluorescent marker gene is downstream of the right genomic recombination junction sequence, and the RNA polymerase promoter is upstream of the left genomic recombination junction sequence.

In various embodiments, step (iv) comprises culturing the eukaryotic host cell in a selection medium under conditions that allows growth of eukaryotic host cell expressing the selection marker gene and subsequently analysing the expression levels of the second fluorescent marker gene, and the first fluorescent marker gene, by using flow cytometry.

In various embodiments, the method further comprises, after step (v), confirming the stable integration of the first circular DNA construct into the landing pad of the eukaryotic host cell, by PCR analysis.

In various embodiments, the method further comprises, after step (v), digesting the genomic DNA of the eukaryotic host cell with one or more restriction enzymes and analysing the digested fragments by using Southern blot.

In various embodiments, the method further comprises isolating the identified eukaryotic host cell comprising a landing pad into which the first circular DNA construct has stably integrated, by serial dilution.

In various embodiments, in the first circular DNA molecule, the selection marker gene is downstream of the lambda integrase recombination partner sequence of attP, and the second fluorescent marker gene is downstream of the selection marker gene.

In various embodiments, the second fluorescent marker gene is comprised in an expression cassette with the constitutive promoter suitable for controlling expression of the second fluorescent marker gene, preferably Chicken β-actin promoter, and optionally the expression cassette comprises a second selection marker gene, preferably a puromycin resistance gene.

In various embodiments, the second circular DNA molecule further comprises a nucleotide sequence encoding for an integration host factor, preferably single chain integration host factor 2 (scIHF2).

In various embodiments, the lambda integrase and integration host factor are comprised in an expression cassette.

In various embodiments, the DNA sequence of interest comprises one or more additional genes.

In various embodiments, the one or more additional genes comprises two genes, wherein the two genes are orientated head-to-tail (CW) or head-to-head (CCW) relative to each other.

In various embodiments, the one or more additional genes comprise monoclonal antibody IgG PD-1 heavy and light chain genes, wherein the two IgG PD-1 genes are orientated head-to-tail (CW) or head-to-head (CCW) relative to each other.

In various embodiments, the expression of genes comprised in the DNA sequence of interest is stable and sustained for at least two weeks in the absence of selection pressure.

In various embodiments, the method further comprises, after step (iv), encapsulating the eukaryotic host cell, preferably using a cellulose sulfate-based encapsulation protocol.

In another aspect, the invention relates to a transgenic eukaryotic host cell or cell line obtained by the methods disclosed herein.

### BRIEF DESCRIPTION OF THE DRAWINGS

Various embodiments of the invention will be better understood with reference to the detailed description when considered in conjunction with the non-limiting examples and the accompanying drawings.

FIG. 1 shows a schematic diagram of the steps for the insertion of the single copy landing pads according to various embodiments, followed by steps for the A-Integrase mediated insertion of a DNA of interest into the landing pad according to various embodiments.

FIG. 2 shows a schematic diagram of the predicted recombination construct and positions of PCR primers: A&B (Left junction) and C&D (Right junction), which flank the resulting hybrid recombination junction attR and attL. Ccba refers to the Chicken beta actin promoter.

FIG. 3 shows the PCR confirmation of attP×attB recombination resulting in attR and attL sites at the left (0.577 kb) and right (1.366 kb) junctions, respectively (lane L—DNA ladder; lane 1—attB-Hygro-EGFP transfected Clone 17 (bulk cells) with primers A and B (0.57 kb product); lane 2—Clone 17 parental control cells with primers A and B; lane 3—Water control with primers A and B; lane 4—Vacant lane; lane 5—Clone 17 parental control with primers C and D; lane 6—attB-Hygro-EGFP transfected Clone 17 (bulk cells) with primers C and D (1.36 kb product); lane 7—Water control with primers C and D).

FIG. 4 shows the DNA sequencing analysis and results confirming that recombination between the genomic attP and attB on the target vector was mediated by Int-C3, and recombination resulted in two hybrid recombination junctions attR and attL. The highlighted section refers to the confirmed recombination junction sequences.

FIG. 5 shows a schematic diagram of the predicted recombination product with a 5.3 kb genomic insertion within the genomic landing pad, with probes complementary to eGFP or mCherry gene to identify DNA fragments digested by restriction enzymes via Southern blotting.

FIG. 6 shows Southern blot analysis of two monoclonal mCherry⁺ lines carry a single copy of the landing pad (lanes 3 and 9) when compared to parental Expi293F cells as negative control (lanes 1 and 7), with recombination between the target vector and genomic landing pad resulted in the expected 5.3 kb genomic fragment (lanes 2 and 8). Controls for the functionality of the probes included linearized attB-hygro-EGFP target vector (lane 4; 7.5 kb) and linearized landing pad-containing mCherry vector (lanes 5 and 6; 5.2 kb).

FIG. 7 shows eGFP expressing cell populations analyzed by flow cytometry at day 0 and day 14 for two monoclonal mCherry lines (clone 17 and 29). Dot plots representing mCherry positive/negative and eGFP positive/negative cell populations. Q1 shows cells expressing mCherry marker; Q2 shows cells expressing both mCherry and GFP; Q3 shows cells which express no fluorescent marker; Q4 shows cells expressing GFP.

FIG. 8 shows the generation of landing-pad inserted clones: (A) An illustration of pEF_attP_mCherry and of the experimental strategy for creating EF_attP_mCherry or landing pad inserted clonal Expi293F cells followed by intC3 facilitated attP×attB recombination and targeted integration of pattB_HygroR_eGFP; (B) Screening of mCherry positive clones. Expi293F cells were sorted for mCherry positive fluorescence after transfection with pEF_attP_mCherry. Once a stable population had been obtained, single cell clones were obtained by dilution. Single cell clones were next analyzed by flow cytometry. Clone 17 (black border) was selected for further experiments.

FIG. 9 shows the targeted integration of pattB_HygroR_eGFP landing pad: (A) Schematic diagram showing predicted recombination construct and positions of PCR primers: 39_EF_fwd and 238_Hygro_rev (Left junction) and 201_ori_fwd and 66_mCherry_rev (Right junction); (B) PCR confirmation of attP×attB recombination resulting in attR and attL sites at the left (0.577 kb) and right (1.366 kb) junctions, respectively. PCR was performed with genomic DNA as a template from a green negative, a green positive colony and clone 17 with the mentioned primers. Genomic DNA from bulk targeted and antibiotic selected cells was used as positive control and no template DNA as water control.

Ladder denotes 1 kb DNA ladder; (C) Flow cytometric analysis of the selected colony. Dot plots representing mCherry negative and eGFP negative Expi293F cells in the lower left quadrant in the first panel, mCherry positive and eGFP negative clone 17 cells in the upper left quadrant (Q1) in the second panel and mCherry negative and eGFP positive cells from green positive colony in the lower right quadrant (Q4) in the third panel.

FIG. 10 shows the DNA sequencing analysis of junction PCRs according to FIG. 9, confirming the site of insertion of pattB_HygroR_eGFP landing pad by IntC3.

FIG. 11 shows EGFP and mCherry expressing cell populations analyzed by flow cytometry, for parental Expi293F cells as negative control, after monoclonal mCherry line (clone 17) cell populations co-transfected with and without IntC3 expression plasmid.

FIG. 12 shows a single copy of the landing pad in chromosome 2 of clone 17 as described in Example 2; (A) Schematic representation of pattB_HygroR_eGFP integrated construct with positions of BsrGI restriction sites and of the ˜5.3 kb predicted product after digestion; (B) Southern blot confirmation of single landing pad site. Southern blot was performed with BsrGI digested genomic DNA from Expi293F, clone 17 and green positive colony cells and incubated with a mCherry gene probe followed by an eGFP gene probe after stripping the same blot. As a positive control 0.5 million copies of pEF_attP_mcherry (5.217 kb) and pattB_HygroR_eGFP (7.550 kb) were used after linearization by BsrGI; (C) Schematic drawing of the landing pad site in the SH3RF3 intron of chromosome 2 of clone 17 depicting Nhel and Agel restriction sites with the primers used for nested PCR after inverse PCR and junction PCR; (D) Detection of the specific site of landing pad insertion by inverse PCR. Inverse PCR and nested PCR were performed, after Nhel (for left junction or EF promoter side) or Agel (for right junction or mCherry side) digestion of clone 17 genomic DNA, with EF_rev_104 and mCherry_fwd_597 primers and nested PCR products were resolved on an agarose gel. DNA bands marked with an arrow were excised and extracted DNA was sequenced to identify the site of the landing pad insertion in the genome; (E) Genomic location confirmation by junction PCR. Junction PCR was performed with clone 17 genomic DNA using C17_gnmc_fwd and 255_pUC_ori_rev primers for the left junction or mCherry_fwd_597 and C17_gnmc_rev primers for the right junction. Amplified products were sequenced to confirm the site of insertion. Ladder denotes 1 kb DNA ladder.

FIG. 13 shows a schematic diagram of the pEF_attP_mCherry cassette integration resulting from a DNA double strand break in the intron with loss of only six nucleotides (AATTCA), and an inverse PCR analysis after genomic DNA digestion with restriction enzymes.

FIG. 14 shows the DNA sequencing analysis of junction PCRs confirming the site of insertion of pEF_attP_mCherry in the SH3RH3 intron and break points in the plasmid. The highlighted sequences indicate the recombination junctions each comprised of plasmid and genomic sequences.

FIG. 15 shows the targeted integration of IgG genes containing plasmids at the landing pad in clone 17: (A) a schematic diagram of attP×attL recombination between the landing pad and anti-PD1 IgG heavy and light chain genes containing plasmid with either CW or CCW orientations. Predicted integrated constructs are depicted with the primers used to confirm integration by junction PCR analysis; (B) PCR confirmation of the left and right junctions. PCR was performed with genomic DNA from different subclones of clones 6, 8, 12, 19 and 23 using either 39_EF_fwd and Amp_rev_498 primers for the left junction with an expected 1.289 kb product or 231_Puro_rev and 66_mCherry_rev primers for the right junction with an expected 1.101 kb product. Bands obtained after resolution on an agarose gel were later confirmed by sequencing. Clones 6B1 and 23A4, marked by black border, were further used for protein expression. Ladder denotes 1 kb DNA ladder.

FIG. 16 shows the analysis of junction PCRs and confirmation of pure cell sub-clones from both target vectors from clones 6 (CW) and 23 (CCW) being obtained, without antibiotic selection. Clones 6 and 23, marked by black border. Ladder denotes 1 kb DNA ladder.

FIG. 17 shows the DNA sequencing analysis of junction PCRs confirming the site of insertion of pattP_HygroR_PD1 landing pad by IntC3.

FIG. 18 shows (A) a schematic diagram of the inserted PD-1 transgene constructs in both orientations (CW or CCW) and PCR sequencing analysis on the two selected sub-clones #6B1 and #23A4 confirming that both clones had the correct internal sequence indicative of their transgene orientations (CW or CCW) without cross-contamination; and (B) cell populations analyzed by flow cytometry of cell sub-clones #17, #6B1 and #23A4 were also analysed by flow cytometry and sub-clones #6B1 and #23A4 were found to be more than 98% single eGFP+(Q4).

FIG. 19 shows IgG expression and purification from landing pad targeted clones: (A) Schematic diagram of IgG genes carrying plasmids, with either CW or CCW arrangement, integrated at the landing pad in clone 17. Sphl restriction sites are shown that would yield a 5.995 kb product after digestion of the genomic DNA clones with both CW or CCW arrangement of the transgenes; (B) Southern blot confirmation of the single copy integration of IgG transgenes for both orientations. Southern blot was performed with Sphl digested genomic DNA from clone 17, 6B1 (CW) and 23A4 (CCW) cells and analyzed with an IgG light chain gene probe. As a positive control, 0.5 million copies of targeting plasmid with CW arrangement (10.793 kb) were used after digestion with Sphl, yielding an 8.468 kb fragment when hydridized to the light chain gene probe; and (C) Purification of secreted IgG. Secreted IgG was purified from both clone 6B1 and 23A4 media by protein G agarose resin and an equal volume (30 μl) from each eluant was resolved on SDS-PAGE.

FIG. 20 shows cell populations analyzed by flow cytometry at day 0 and day 14 for Expi293F cells and sub-clones #17, #6B1 and #23A4: (A) flow cytometry of cells from a pattB_HygroR_eGFP targeted colony before and after 14 days of continuous culture were found to maintain homogeneity at >90%; and (B) FACS analysis showing sustained and homogenous (>98% eGFP+) in PD-1 antibody expressing sub-clones #6B1 and #23A4 after 14 days of culture without selection.

FIG. 21 shows secretion of IgG from encapsulated clone 6B1 cells: (A) Representative image of capsule 4 in bright field and fluorescence showing homogenous eGFP expression in encapsulated cells. Encapsulation of clone 6B1 cells. Cells were encapsulated in Cell-in-a-Box® and transferred to a 24-well plate with one capsule per well. This was followed by visualization under a fluorescence microscope 2 weeks after encapsulation. Scale bars represent 1 mm at 4× magnification; and (B) Measurement of secreted IgG from capsule 4. 20 days after encapsulation, the media in the well with capsule 4 was replaced with fresh media, which was defined as Day 0. Samples were then collected on Day 0, Day 2 and Day 3. The concentration of secreted IgG was estimated by ELISA in triplicate. Data presented here is the mean with standard deviation.

FIG. 22 is a schematic diagram showing that Cre and Flp recombinases expressed from transfected mRNA can excise undesirable DNA segments. For simplicity, a promoter operably linked to the eGFP is not shown, but would be understood to be included in practice.

FIG. 23 shows a graphical schematic diagram of the process steps in creating cellulose-based mini-bioreactors using the engineered human cell lines.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” “About”, as used herein in connection with numerical values refers to the referenced numerical value ±10% or ±5%. In case of conflict, the present specification, including explanations of terms, will prevail.

Reliable cell-based platforms to test and/or produce biologics in a sustainable manner are important for the biotech industry. Conventional strategies and tools for Chinese Hamster Ovary master cell engineering have been developed over thirty years, whereby most genome engineering approaches that are aimed to increase the production of biotherapeutics have been applied to CHO cells. Accordingly, there is still a void when it comes to the generation of alternative master cell lines, preferably eukaryotic cell lines, for a number of biopharmaceutical and biomedical applications, e.g. cell therapies with encapsulated mini bioreactors.

The present application provides a novel, general strategy for establishing eukaryotic cell lines which contain a single copy genomic landing pad for use in methods for A-Integrase-mediated site-specific DNA recombination and transgene insertion.

Utilizing λ integrase, a sequence-specific DNA recombinase, a versatile transgenesis platform has been developed for site-specific transgenesis involving a fully characterized single genomic locus as a single copy artificial landing pad for λ-integrase-mediated transgene insertion in a host cell. The single genomic landing pad host cell line was generated to be effectively and safely targeted with multi transgene constructs at a ‘safe harbour site’. Landing pad host cells that have been successfully targeted and transgene inserted, can be easily selected due to a phenotypic change. In this regard, the genomic landing pads disclosed herein are of great value to generate tester master cell lines as well as in the context of safe mini bioreactors for in vivo cell therapies.

Methods for Producing a Eukaryotic Cell and/or Cell Line for λ-Integrase-Mediated Recombination

Accordingly, in various embodiments there are provided methods for producing a eukaryotic cell and/or cell line for λ-integrase-mediated recombination and transgene insertion, said cell line being obtained from a eukaryotic host cell comprising a landing pad stably integrated into the eukaryotic host cell's genome for λ-integrase-mediated site-specific recombination of a DNA sequence of interest.

The methods disclosed herein comprise the step of transfecting a bacterial plasmid into a eukaryotic host cell at suitable conditions to induce said transfection, wherein the bacterial plasmid comprises a landing pad construct or cassette.

The term “transfection” as used herein means the uptake of exogenous or heterologous RNA or DNA by a cell. A cell has been “transfected” by exogenous or heterologous RNA or DNA when such RNA or DNA has been introduced inside the cell.

The term “host cell” as used herein refers to a living cell into which a DNA sequence of interest is to be or has been introduced. The living cell includes both a cultured cell and a cell within a living organism. In various embodiments, host cells can be engineered to incorporate a desired gene on its chromosome or in its genome. The method described herein may be performed in all eukaryotic host cells. In various embodiments, the eukaryotic host cell is a higher eukaryotic host cell. The term “higher eukaryotic cell” as used herein refers to eukaryotic cells that are not cells from unicellular organisms. In other words, a higher eukaryotic cell is a cell from (or derived from, in case of cell cultures) a multicellular eukaryote such as a human cell line or another mammalian cell line. Particularly, the term generally refers to mammalian cells, human cell lines and insect cell lines. More particularly, the term refers to vertebrate cells, even more particularly to mammalian cells or human cells.

In various embodiments, the eukaryotic host cell is a mammalian cell. The mammalian cell lines can include, but are not limited to a human, simian, murine, mice, rat, monkey, rabbit, rodent, hamster, goat, bovine, sheep or pig cell lines. In various embodiments, the eukaryotic host cell is a human cell. In various embodiments, the eukaryotic host cell is a cell from a cell line including, but are not limited to Chinese hamster ovary (CHO) cells, murine myeloma cells such as NSO and Sp2/0 cells, COS cells, Hela cells and human embryonic kidney (HEK-293) cells or derivatives thereof.

In various embodiments, the eukaryotic host cell is a human embryonic kidney (HEK-293) cell, more preferably a human Expi293F cell. In particular, Expi293F cells may be used as transgenic master cell lines with modular features as a basis for biopharmaceutical testing/production and innovative therapeutic applications such as transplantable cell-encapsulated mini bioreactors.

The term “bacterial plasmid” as used herein refers to a circular DNA molecule capable of replication in a bacterial host cell. A bacterial plasmid may contain an appropriate origin of replication (ori), which is a sequence of DNA sufficient to enable the replication of the plasmid in a host bacterial cell and a bacterial backbone sequence. A bacterial plasmid may also contain a selectable marker sequence within said bacterial backbone sequence, which encodes a selectable marker conferring cellular resistance to antibiotics such as ampicillin, kanamycin, chloramphenicol, and tetracycline. In various embodiments, the bacterial plasmid encodes a selectable marker conferring cellular resistance to ampicillin (i.e. AmpR sequence). In various embodiments, the method disclosed herein may further comprise providing a bacterial plasmid comprising the landing pad construct and a bacterial backbone sequence.

The term “landing pad” as used herein refers to a nucleic acid sequence or construct that allows for site-specific recombination with another genetic element, such as a plasmid or vector, mediated by a lambda (λ) integrase. The landing pad generally functions as the integration site for a DNA sequence of interest, and optionally the corresponding regulatory factors, into the genetic locus of a host cell.

In various embodiments, the landing pad may comprise a RNA polymerase promoter, a lambda (λ) integrase recombination sequence and a fluorescent marker gene.

In various embodiments, the landing pad may comprise from 5′ to 3′ direction: the RNA polyrmerase promoter, the lambda (λ) integrase recombination sequence and the fluorescent marker gene, such that the lambda (λ) integrase recombination sequence is flanked by the other two elements with the RNA polymerase promoter being positioned upstream (towards the 5′ direction) of the lambda (λ) integrase recombination sequence, and the fluorescent marker gene being positioned downstream (towards the 3′ direction) of lambda (λ) integrase recombination sequence.

The term “downstream” as used herein refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription. The term “immediately downstream” may be used to specify that the nucleotide sequence immediately follows and is directly next to the reference nucleotide sequence in the 3′ direction, with no other intervening nucleotide sequence or genetic element therein between. The term “upstream” as used herein refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription. The term “immediately upstream” may be used to specify that the nucleotide sequence immediately precedes and is directly next to the reference nucleotide sequence in the 5′ direction, with no other intervening nucleotide sequence or genetic element therein between.

In various embodiments, the “RNA polymerase promoter” is any promoter that is highly capable and efficient at initiating transcription and producing a large amount of RNA from a gene and corresponding proteins, and may be termed as a “strong promoter”. Specifically, the term “RNA polymerase promoter” refers to a region of DNA that may be upstream from the start of transcription, and that may be involved in recognition and binding of RNA polymerase and other proteins to initiate transcription. The “RNA polymerase promoter” may be derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters. Many promoters known in the art can be used for expression in host cells. Examples include, but are not limited to, the promoter of the mouse metallothionein I gene sequence; the TK promoter of Herpes virus; the SV40 early promoter: the yeast gall gene sequence promoter, the CMV promoter, the EF-1 promoter, the actin promoter, the phosphoglycerate kinase promoter, the ubiquitin promoter and the thymidine kinase promoter, the ecdysone-responsive promoter(s), tetracycline-responsive promoter, and the like.

In various embodiments, the RNA polymerase promoter is a viral RNA polymerase promoter selected from a CMV promoter, SV40 promoter or a TK promoter.

In various embodiments, the RNA polymerase promoter is a eukaryotic RNA polymerase promoter. In various embodiments, the eukaryotic RNA polymerase promoter may be a eukaryotic RNA polymerase II promoter, more preferably a EF-1α promoter.

Lambda integrase recombination sequences are recombination substrates for the lambda integrase and may be referred to as att sequences or sites. The term “lambda integrase” as used herein refers to any lambda-derived integrases that possess endonuclease and ligase activities. The lambda integrase may be referred to as a bacteriophage lambda integrase. As known in the art, the phage lambda integrase belongs, like Cre and Flp, to the integrase family of the sequence-specific conservative DNA recombinases and catalyses the integrative recombination between two different recombination att sites. An attsequence is the recognition site where binding, cleavage, and strand exchange are performed by the lambda integrase and any associated accessory proteins thereof. The landing pads disclosed herein contain recombination sequences for lambda Integrase or functional variants that utilize corresponding recombination partner sequences comprised in circular DNA molecules, such as vectors, for the site-specific insertion of a DNA sequence of interest into said genomic landing pads. The att sequences or sites may include but are not limited to attB, attP, attL and attR.

AttB comprises 21 nucleotides and was originally isolated from the E. coli genome (Mizuuchi, M. and Mizuuchi, K. (1980) Proc. Natl. Acad. Sci. USA, 77, pp. 3220). On the other hand attP having 243 nucleotides is much longer and occurs naturally in the genome of the bacteriophage lambda (Landy, A. and Ross, W. (1977) Science, 197, pp. 1147). The recombination between attB and attP leads to the formation of two new recombination sequences, namely attL and attR, which may serve as substrate and recognition sequences for a further recombination reaction, the excision reaction. A comprehensive summary of the bacteriophage lambda integration is given in Landy, A. (1989) Annu Rev. Biochem., 58, pp. 913.

Respective pairs of these att sites (i.e. partner sequences) may be selected from attP, attB, attR and attL. In various embodiments, the pairs of att sites may be selected from attP and attB, attP and attL, or attB and attB.

Furthermore, recombination-competent derivatives of the att sites may also be selected for use in the methods disclosed herein. In this regard, the methods disclosed herein may be carried out not only with the naturally occurring (i.e. wild-type) attB, attP, attL and/or attR sequences but also with derivatives of attB, attP, attL and/or attR sequences. In various embodiments, the attP sequence may comprise or consist of a nucleotide sequence according to SEQ ID NO:1 or a derivative thereof, the attB sequence may comprise or consist of a nucleotide sequence according to SEQ ID NO:2 or a derivative thereof, the attL sequence may comprise or consist of a nucleotide sequence according to SEQ ID NO:3 or a derivative thereof, and the attR sequence may comprise or consist of a nucleotide sequence according to SEQ ID NO:4 or a derivative thereof.

The term “wild-type” or “naturally occurring” as used herein, has the meaning commonly understood by those skilled in the art, which means a typical form of organisms, strains, genes, or features that distinguish it from mutants, derivatives or variant forms when it exists in nature, it can be isolated from natural sources and has not been deliberately modified.

The term “derivative” as used herein relates to attB, attP, attL and attR sequences having one or more substitutions, preferably two, three, four, five or six in the overlap region and/or core region in contrast to naturally occurring (i.e. wild-type) attB, attP, attL and attR sequences. The term “derivative” relates to any functional fragments thereof and nucleotide sequences in eukaryotic cells supporting sequence-specific recombination, e.g. at/H identified in the human genome. The term “derivative” in general includes sequences derived from attB, attP, attL or attR sequences suitable for realizing the intended use of the present invention, which means that the derivative sequences mediate sequence-specific recombination events driven by the lambda integrase (wild-type or mutants).

In various embodiments, the landing pad comprises a single lambda (λ) integrase recombination sequence. In various embodiments, the lambda integrase recombination sequence is modified to remove ATG start codons.

In various embodiments, the single lambda integrase recombination sequence in the landing pad is an attP sequence or a derivative thereof, wherein the attP sequence has a 5′-3‘ POP’ structure with P and P′ describing the left and right arms at the attP site and O describes the homologous core.

In various embodiments, the lambda integrase recombination sequence attP is modified to remove ATG start codons, that is, all ATG start codons are removed in the sequence of attP. As such, in various embodiments, the landing pad comprises a modified lambda integrase recombination sequence attP that lacks an ATG start codon, or derivatives thereof which also lack an ATG start codon. In various embodiments, the modified lambda integrase recombination sequence attP or derivative thereof lacks an ATG start codon in the (P—O—P′) nucleotide sequence. Advantageously, the lack of these translational ATG start codons allows for the expression of the fluorescent marker gene from the upstream RNA polymerase promoter of the landing pad.

In various embodiments, the modified lambda integrase recombination sequence attP comprises or consists of the nucleotide sequence set forth in SEQ ID NO: 5 or derivatives thereof.

In various embodiments, the landing pad may also comprise at least one additional recombination site (e.g., loxP or FRT or attP site) that is compatible with a recombinase other than lambda integrase, such as Cre or Flp or Bxb1 recombinases, respectively. Accordingly, in various embodiments, the landing pad may additionally comprise a loxP, a FRT, and/or an attP site alone or in combination for Cre, Flp, or Bxb1 recombinases, respectively. In particular, a loxP and/or a FRT site may flank the promoter and fluorescent marker gene of the landing pad, such that Cre and Flp recombinases can then excise undesirable DNA segments following transgene integration. Both Cre and Flp can catalyse intermolecular recombination, and may recombine a circular DNA molecule carrying a copy of their respective recombination sequences with a corresponding recombination sequence in the landing pad. In various embodiments, the landing pad comprises both a loxP and FRT sequence, whereby the FRT sequence is upstream of the promoter and the loxP sequence is downstream of the fluorescent marker gene.

The term “fluorescent marker gene” disclosed herein refers to a gene or nucleotide sequence whose expression in a transfected host cell can be detected or made visible. In various embodiments, the fluorescent marker gene is operably linked to the RNA polymerase promoter, and is selected from green fluorescent protein (GFP) or enhanced green fluorescent protein (eGFP), yellow fluorescent protein (YFP) or enhanced yellow fluorescent protein (eYFP), red fluorescent protein (RFP), mCherry, mRaspberry, mPlum, mTomato, dsRed, and luciferase.

In various embodiments, the fluorescent marker gene is operably linked to the RNA polymerase promoter. In various embodiments, the fluorescent marker gene is mCherry and is operably linked to the RNA polymerase promoter.

The term “operably linked” as used herein refers to the relationship between two or more nucleotide sequences that interact physically or functionally. For example, a promoter or regulatory nucleotide sequence is said to be operably linked to a nucleotide sequence that codes for an RNA or a protein if the two sequences are situated such that the regulatory nucleotide sequence will affect the expression level of the coding or structural nucleotide sequence. “Regulatory nucleotide sequences” as used herein refer to nucleotide sequences that influence the timing and level/amount of transcription, RNA processing or stability, or translation of the associated coding sequence. Regulatory sequences may include promoters; translation leader sequences; introns; enhancers; 25 stem-loop structures; repressor binding sequences; termination sequences; and polyadenylation recognition sequences. Particular regulatory sequences may be located upstream and/or downstream of a coding sequence operably linked thereto.

In various embodiments, the landing pad further comprises regulatory sequences to enhance the expression of the fluorescent marker gene, such as polyA sequence, T2A coding sequence, IRES (Internal Ribosome Entry Site), etc. In various embodiments, the landing pad further comprises a poly(A) sequence comprising 20 to about 400 adenosine nucleotides. In various embodiments, the poly(A) sequence is downstream of the fluorescent marker gene.

Accordingly, in various embodiments, the landing pad corprises or consists of:
a RNA polymerase promoter;
a modified lambda integrase recombination sequence attP or derivative thereof, downstream of the promoter, wherein the modified lambda integrase recombination sequence has been modified to remove ATG start codons, preferably the ATG start codons are removed in the 5′-3′ (P—O—P′) nucleotide sequence of attP; and
a first fluorescent marker gene, downstream of the modified lambda integrase recombination sequence and operably linked to the promoter.

In various embodiments, the ianding pad comprises or consists of:
an EF-1α promoter;
a modified lambda integrase recombination sequence attP comprising or consisting of the nucleotide sequence set forth in SEQ ID NO: 5 or derivatives thereof; and
a mCherry gene, downstream of the modified lambda integrase recombination sequence attP and operably linked to the EF-1α promoter.

The preparation and provision of the bacterial plasmid comprising the landing pad disclosed herein, as well as methods to transfect eukaryotic host cell with the bacterial plasmids for integrating the landing pad into the genome of the host cell are well known in the art and can be routinely applied by those skilled in the art. Transfection conditions, reagents and mediums can be selected by those skilled in the art based on the host organism used by resorting to general knowledge and techniques known in the art.

In various embodiments, the eukaryotic host cell may be adherent or non-adherent. That is, the eukaryotic host cell may be adherent cells that naturally adhere to a solid substrate, or may be non-adherent cells that may be maintained as cells in a suspension of freely growing cells by cultivation in an appropriate cell culture system. In various embodiments, where the host cell is a non-adherent host cell, the transfection step may comprise adding a mix of Lipofectamine and the plasmid to the host cells and incubated overnight under antibiotics free growth medium. In various embodiments, where the host cell is an adherent host cell, the adherent host cell may be initially grown to a desired confluence level (e.g. 70-90%) and then adding a mix of Lipofectamine and the plasmid to the host cells and incubated under antibiotics free growth medium.

In various embodiments, the bacterial plasmid carrying the landing pad disclosed herein is randomly integrated into the genome of the eukaryotic host cell which may be mediated by internal cellular recombination events. However, it is also possible to integrate the landing pad by any other mechanisms into the genome of the eukaryotic host cell such as homologous recombination mediated by programmable designer nucleases (TALEN, ZincFingers, or CRISPR-Cas). Integration of said bacterial plasmid can also be achieved via sequence-specific recombination using sites different from those being integrated, e.g., by using loxP/FRT sequences. As will be readily appreciated by the skilled person, at least the landing pad (i.e. promoter, recombination site and fluorescent marker) may be integrated into the genome of the eukaryotic host cell, whereby the presence or absence of the rest of the bacterial plasmid and genetic elements (other than those of the landing pad) may or may not be also integrated.

In various embodiments, the bacterial plasmid, or at least the landing pad, may be integrated into an intron sequence of the host cell genome. For example, in various embodiments wherein the eukaryotic host cell is a human Expi293F cell, the bacterial plasmid, or at least the landing pad, may be integrated into the third intron of the SH3 Domain Containing Ring Finger 3 gene (SH3RF3) on chromosome 2.

However, it will be appreciated that the bacterial plasmid may be integrated into the third intron of the SH3 Domain Containing Ring Finger 3 gene (SH3RF3) on chromosome 2 in eukaryotic, preferably human, cells or cell lines other than those derived from human Expi293F.

The method disclosed herein further comprises the step of cultivating or culturing the transfected host cell under conditions that allow expression of the fluorescent marker gene comprised in the landing pad.

Methods for culturing the transfected host cells are well-known in the art and can be routinely applied by those skilled in the art. Culturing conditions, reagents and mediums can be selected by those skilled in the art based on the host organism used by resorting to general knowledge and techniques known in the art. In various embodiments, the culturing step comprises incubating the transfected host cell in expression, suspension and/or growth media to allow expression of the fluorescent marker gene.

In various embodiments, the culturing step is carried out for at least two days.

In various embodiments, the culturing step comprises passaging the transfected eukaryotic host cell one or more times in the absence of selection pressure, whereby the transfected eukaryotic host cell is cultured in an antibiotic-free media. In other words, the transfected host cell is not cultured and grown in a selection medium and is not cultured in the presence of a selection agent, such as an antibiotic.

The method disclosed herein further comprises the step of identifying a eukaryotic host cell into which the bacterial plasmid has stably integrated based on the detectable expression of the first fluorescent marker gene. In particular, the step of identifying comprises selecting a host cell into which at least the landing pad (i.e. promoter, recombination site and fluorescent marker) has stably integrated, whereby the presence or absence of the rest of the plasmid and genetic elements (other than those of the landing pad) may or may not be also integrated in the host cell genome.

The expression level of the fluorescent marker gene may be correlated with the transcription activity at the genomic site of integration of the landing pad, whereby cells showing a high expression level at site of integration, cell robustness, and good growth characteristics, e.g., in a bioreactor, can be identified effectively. The level of expression of the fluorescent marker gene can be determined by methods well known in the art. For example, mRNA transcribed from the introduced gene sequence can be quantified by Northern blot hybridization, ribonuclease RNA protection, in situ hybridization to cellular RNA or by PCR. Proteins encoded by a selected sequence can be quantified by various methods, e.g., by ELISA, by Western blotting, by radioimmunoassays, by immunoprecipitation, by assaying for the biological activity of the protein, by immunostaining of the protein followed by FACS analysis. By such a method desirable candidates of a cell line for subsequent insertion of a DNA sequence of interest may be obtained.

As will be appreciated, in various embodiments where the bacterial plasmid, or at least the landing pad has been randomly integrated into the host cell's genome, the result is a heterogenous and diverse population of cells, necessitating a screening process to identify and isolate a suitable single cell clone carrying a single genomic copy of the landing pad disclosed herein and exhibits desirable expression levels of the fluorescent marker gene.

Accordingly, the expression of the fluorescent marker gene allows the screening and detection of successful integration of the landing pad into the eukaryotic host cell genome, whereby the fluorescent marker gene is operably linked to the upstream RNA polyrnerase prornoter. Expression of the fluorescent marker gene identifies a host cell carrying the landing pad construct. Accordingly, the fluorescent marker gene functions in providing an applicable screening strategy for detecting the integration of functional single copy landing pads into the cell genome for A-Integrase.

In various embodiments, the expression of the fluorescent marker gene may be detected and optionally quantified by flow cytometry. More particularly, eukaryotic host cells with the bacterial plasmid integrated into the genome is identified by fluorescently activated cell sorting (FACS). In various embodiments, FACS may be repeated at seven-day intervals. Based on the detected and optionally quantified expression levels of the fluorescent marker gene, a eukaryotic host cell expressing the fluorescent marker gene may be obtained.

In various embodiments, the identified eukaryotic host cell may be comprised in a bulk cell population of eukaryotic host cells expressing the fluorescent marker gene. In various embodiments, eukaryotic host cells expressing the fluorescent marker gene may be enriched by repeating the fluorescently activated cell sorting (FACS) of the bulk cell population three or more times.

In various embodiments, the population of eukaryotic host cells obtained exhibit a homogenous expression level of the fluorescent marker gene as determined by flow cytometry analysis. In this regard, eukaryotic host cells exhibiting homogenous expression levels of the fluorescent marker gene, indicate the presence of a single population of cells with a single copy of the landing pad. In contrast, host cells exhibiting two or more distinct expression levels of the fluorescent marker gene, indicate the presence of two or more populations of host cells with distinct fluorescent marker gene expression levels with a different copy number of the landing pad.

In various embodiments, where the eukaryotic host cell is a non-adherent cell, the method disclosed herein may further comprise, after the identifying step, culturing the identified eukaryotic host cells in an adherent cell culture media such that the eukaryotic host cells are adapted to adherent growth. In various embodiments, where the host cell is an adherent cell, the method disclosed herein may further comprise, after the identifying step, culturing the identified host cells in an adherent cell culture media.

The method disclosed herein further comprises the step of isolating the identified eukaryotic host cells for generating a eukaryotic cell line comprising the landing pad for lambda integrase-mediated recombination. In various embodiments, the eukaryotic cell line is a monoclonal cell line.

In various embodiments, the isolating step comprises serial dilution of the cell population of eukaryotic host cells identified as expressing the fluorescent marker gene, more preferably expressing a homogenous expression level of the fluorescent marker gene. In various embodiments, the serial dilution may result in the obtaining of a monoclonal eukaryotic host cell line comprising the genomic landing pad for λ-integrase-mediated recombination.

Methods of serially diluting the cell population of identified host cells are well-known in the art and can be routinely applied by those skilled in the art. Diluting conditions, reagents and mediums can be selected by those skilled in the art based on the host organism used by resorting to general knowledge and techniques known in the art. In particular, the adherent or suspension cell culture of the bulk cell population of eukaryotic host cells identified as expressing the fluorescent marker gene may be serially diluted to obtain single cell colonies that may be expanded to generate a homogenous population of a monoclonal cell line comprising the landing pad, more preferably a single genomic copy of the landing pad, for λ-integrase-mediated recombination.

In various embodiments, the isolating step comprises single cell FACS of eukaryotic host cells identified as expressing the fluorescent marker gene, more preferably expressing a homogenous expression level of the fluorescent marker gene. In various embodiments, the single cell FACS may result in the obtaining of a monoclonal eukaryotic host cell line comprising the genomic landing pad for λ-integrase-mediated recombination.

Accordingly, in various embodiments, there is provided a method for production of a eukaryotic cell and/or cell line comprising a genomic landing pad for λ-integrase-mediated recombination, the method comprising:
(i) transfecting a bacterial plasmid into a eukaryotic host cell at suitable conditions to induce said transfection, wherein the bacterial plasmid comprises a landing pad comprising:
   a. an RNA polymerase promoter, preferably a eukaryotic RNA polymerase promoter, more preferably EF-1α promoter;
   b. a modified lambda integrase recombination sequence attP or derivatives thereof, downstream of the promoter, wherein the modified phage lambda integrase recombination sequence has been modified to remove ATG start codons; and
   c. a first fluorescent marker gene, preferably mCherry, downstream of the modified lambda integrase recombination sequence and operably linked to the promoter;
(ii) culturing the transfected eukaryotic host cell under conditions that allow expression of the first fluorescent marker gene;
(iii) identifying a eukaryotic host cell into which the bacterial plasmid has stably integrated based on the detectable expression of the first fluorescent marker gene, preferably by using flow cytometry; and
(iv) isolating the identified eukaryotic host cell to obtain the eukaryotic cell or cell line comprising the genomic landing pad for λ-integrase-mediated recombination.

As a result of the methods disclosed herein, a eukaryotic cell or cell line comprising a genomic landing pad for λ-integrase-mediated recombination, can be produced in the absence of selection pressure, whereby the method does not comprise a step of culturing, growing or maintaining the host cell in a selection medium and is not cultured in the presence of selection agent, such as an antibiotic.

In various embodiments, the isolated eukaryotic host cells may be screened for competency of A-integrase-mediated recombination. In particular, the eukaryotic host cell and/or cell line isolated may be expanded and screened to confirm the stable genomic integration of the landing pad using specific PCR primer combinations and PCR sequence analysis based on the sequences of the promoter and fluorescent marker gene. In particular, isolated single cells or cell colonies may be expanded to generate a homogenous population of a monoclonal cell line comprising the landing pad. The single cells or cell colonies may be harvested and grown in 24-well culture plates and then expanded to 6-well plates, Genomic DNA may then be isolated from these expanded cell clones and checked for integration of the landing pad into the host cell's genome.

Primer pairs used may be designed using conventional methods known in the art, based on the sequences of the promoter and fluorescent marker gene of the landing pad, with the resulting amplified products being sequenced to confirm the site of insertion.

In various embodiments, the Genomic DNA of the isolated and expanded eukaryotic host cells may be purified and digested by means of an endonuclease activity, preferably by means of one or more restriction enzymes. A restriction enzyme is an enzyme that cuts DNA at or near specific recognition nucleotide sequences known as restriction sites. Restriction enzymes can be used in the laboratory e.g. to cleave DNA molecules into smaller fragments for molecular cloning and gene characterization.

Restriction enzymes are commonly classified into four types, which differ in their structure and whether they cut their DNA substrate at their recognition site, or if the recognition and cleavage sites are separate from one another. To cut DNA, all restriction enzymes make two incisions, one per each sugar-phosphate backbone (i.e. each strand) of the DNA double helix. A restriction site, also called restriction enzyme recognition site, is a nucleotide sequence recognized by a restriction enzyme. A restriction site is typically a short, preferably palindromic nucleotide sequence, e.g. a sequence comprising 4 to 8 nucleotides. A restriction site is preferably specifically recognized by a restriction enzyme. Recognition sequences in DNA differ for each restriction enzyme, producing differences in the length, sequence and strand orientation (5′ end or the 3′ end) of a sticky-end “overhang” of an enzyme restriction. The choice of the endonuclease and the isolation method to apply in the context of the methods disclosed herein are within the knowledge of the person of average skill in the art.

In various embodiments, the restriction enzymes, may include, but are not limited to Nhel, HindIII, BgrGl, Pvul, SgrAI, Bsml, Bmtl, Agel, EcoRI and Mfel. In various embodiments, the restriction enzymes are Agel and Nhel, whereby the promoter comprises a single restriction site for Agel and the fluorescent marker gene a single restriction site for Nhel.

Accordingly, in various embodiments, the method further comprises isolating and digesting the genomic DNA of the eukaryotic host cell with one or more restriction enzymes with the digested products amplified by PCR.

In various embodiments, the digested genomic DNA of the eukaryotic host cell may be anlysed by using using specific PCR primer combinations and PCR sequence analysis. In particular, primer pairs used were designed with respect to the promoter and fluorescent marker gene with the resulting amplified products being sequenced to confirm the site of insertion. The PCR may comprise inverse and/or nested PCR. In various embodiments, the PCR amplified products may be resolved by electrophoresis on agarose gels and amplified bands sequenced.

In particular, after digestion and PCR, amplified products may be obtained which contains flanking genomic sequences located 5′ of the promoter and 3′ of the integrated fluorescent marker gene coding sequence. Sequencing of these PCR products reveal the genomic locus of the integration site of the landing pad. Subsequent nucleotide sequence alignments may be carried out to further reveal the genomic locus of the landing pad.

In addition, Southern blotting may be employed to analyse the eukaryotic host cell and/or cell line isolated. In various embodiments, the digested genomic DNA of the eukaryotic host cell may be analysed by Southern blotting. The Southe^{r}n blot analysis may confirm a single copy of the genomic landing pad is present in the eukaryotic host cell. In particular, the digested genomic DNA of the eukaryotic host cell may be incubated with a probe for the fluorescent marker gene. As will be appreciated, suitable probes may be designed and prepared using well-known techniques and the knowledge of the skilled person. For example, an mCherry probe (fluorescent marker gene) may be generated using mCherry probe fwd and rev primers. The probe-target hybrids on the Southern blots may be detected by a chemiluminescent assay. A single genomic copy of the landing pad after random integration is indicated by a single restriction band in Southern blots.

Accordingly, there is also provided a eukaryotic host cell or cell line, preferably a monoclonal eukaryotic cell line, obtained by the methods disclosed herein.

In various embodiments, the eukaryotic host cell exhibits a homogenous expression level of the fluorescent marker gene as determined by flow cytometry analysis. In various embodiments, the eukaryotic host cell or cell line comprises a single genomic copy of the landing pad. In various embodiments, the eukaryotic host cell line exhibits homogenous and stable long-term expression levels of the fluorescent marker gene, preferably confirmed by flow cytometry analysis, in the absence of selection pressure. In this context, “long-term” refers to a period of at least two weeks up to one or more months, where expression levels are sustained in the absence of selection pressure.

In particular, in various embodiments, there is provided a eukaryotic host cell or cell line comprising a genomic landing pad for λ-integrase-mediated recombination, wherein the genomic landing pad comprises:
a. a RNA polymerase promoter, preferably an eukaryotic RNA polymerase promoter, more preferably EF-1α promoter;
b. a modified lambda integrase recombination sequence attP or derivatives thereof, downstream of the promoter, wherein the modified phage lambda integrase recombination sequence has been modified to remove ATG start codons; and
c. a first fluorescent marker gene, preferably mCherry, downstream of the modified lambda integrase recombination sequence and operably linked to the promoter.

All embodiments disclosed above in relation to the methods disclosed herein for the production of a eukaryotic cell comprising a genomic landing pad for λ-integrase-mediated recombination, similarly apply to the eukaryotic host cell disclosed herein or eukaryotic host cell line obtained therefrom, and vice versa.

### Methods for λ-Integrase-Mediated Insertion

The eukaryotic host cell or cell line containing the landing pad integrated in to the genome of said cell/cell line may be referred to herein as “landing pad eukaryotic cell or cell line”.

As will be appreciated, the landing pad eukaryotic cell or cell line may then be used in the following described methods or may be generally used for λ-integrase-mediated insertion, particularly transgene insertion, as described herein. Such uses thus also form part of the present invention.

Accordingly, in various embodiments, there is also provided methods for λ-integrase-mediated insertion of a DNA sequence of interest into the landing pad eukaryotic host cell or cell line disclosed herein.

The term “DNA sequence of interest” as used herein refers to any DNA sequence, the manipulation of which may be deemed desirable for any reason (e.g., conferring improved qualities and/or quantities, expression of a protein of interest in a host cell, expression of a ribozyme), by one of ordinary skill in the art. Such DNA sequences include, but are not limited to, coding sequences of structural genes (e.g., reporter genes, fluorescent marker gene, selection marker genes, oncogenes, drug resistance genes, growth factor genes), and non-coding sequences which do not encode an mRNA or protein product (e.g., promoter sequences, polyadenylation sequences, termination sequences, enhancer sequences, small interfering RNAs, short hairpin RNAs, antisense RNAs, microRNAs, long non-coding RNAs).

The DNA sequence of interest comprise genes, or transgenes, which may or may not be operably linked to one or more expression control sequences, such as a promoter, an enhancer, an operator, a termination signal, a 3′-UTR, or a 5′-UTR, an insulator.

In various embodiments, the DNA sequence of interest may comprise one or more selection marker genes. In various embodiments, the DNA sequence of interest may comprise two selection marker genes. In various embodiments, the DNA sequence of interest may comprise a promoter-free selection marker gene that is not operably linked to a promoter.

The term “selection marker gene” as used herein refers to a gene that only allows cells carrying the gene to be specifically selected for or against in the presence of a corresponding selection agent. For example, selectable genes commonly used with eukaryotic cells include the genes for aminoglycoside phosphotransferase (APH), hygromycin phosphotransferase (HYG), dihydrofolate reductase (DHFR), thymidine kinase (TK), glutamine synthetase, asparagine synthetase, and genes encoding resistance to neomycin (G418), hygromycin, puromycin, histidinol D, bleomycin and phleomycin.

In various embodiments, the DNA sequence of interest may comprise a first selection marker gene, preferably a hygromycin resistance gene. The hygromycin resistance gene may be promoter-free and not operably linked to a promoter.

In various embodiments, the DNA sequence of interest may comprise a fluorescent marker gene that is different to the fluorescent marker gene comprised in the landing pad. In this regard, the fluorescent marker gene comprised in the landing pad may be termed as a first fluorescent marker gene, and the fluorescent marker gene comprised in the DNA sequence of interest may be termed as a second fluorescent marker gene. In various embodiments, the second fluorescent marker gene is selected from green fluorescent protein (GFP) or enhanced green fluorescent protein (eGFP), yellow fluorescent protein (YFP) or enhanced yellow fluorescent protein (eYFP), red fluorescent protein (RFP), mCherry, mRaspberry, mPlum, mTomato, dsRed, and luciferase. In various embodiments, the second fluorescent marker gene is green fluorescent protein (GFP) or enhanced green fluorescent protein (eGFP).

In various embodiments, the second fluorescent marker gene is operably linked to a constitutive promoter suitable for controlling expression of the second fluorescent marker gene. In this regard, the DNA sequence of interest may comprise a second fluorescent marker gene and a constitutive promoter.

Many constitutive promoters are known in the art and can be used for expression in host cells of genes and particularly reporter genes. Examples of constitutive promoters include, but are not limited to, the promoter of the mouse metallothionein I gene sequence: the TK promoter of Herpes virus; the SV40 early promoter: the yeast gall gene sequence promoter, the CMV promoter, the EF-1 promoter, the actin promoter, the phosphoglycerate kinase promoter, the ubiquitin promoter and the thymidine kinase promoter, the ecdysone-responsive promoter(s), tetracycline-responsive promoter, and the like. In various embodiments, the constitutive promoter is a CMV promoter, SV40 promoter, EF-1 promoter, or an actin promoter. In various embodiments, the constitutive promoter is an actin promoter, preferably a Chicken β-actin promoter. In various embodiments, the constitutive promoter is the same or different to the RNA polymerase promoter of the landing pad.

In various embodiments, the second fluorescent marker gene and constitutive promoter may be comprised in an expression cassette. In various embodiments, the first selection marker gene, is positioned upstream of the expression cassette, wherein the second fluorescent marker gene is positioned upstream of the constitutive promoter.

In various embodiments, the selection marker gene (i.e. first selection marker gene) and the expression cassette are arranged in opposite orientations to one another. The term “orientation” as used herein in connection with expression cassettes, refers to the directional characteristic of a given cassette or structure. For example, arrows illustrated in the figures disclosed herein indicate the direction of transcription of the elements. In various embodiments, the constitutive promoter is positioned at the 3′ end of the expression cassette, and transcription of the encoding nucleic acid sequence runs from the 3′ terminus to the 5′ terminus of the sense strand, making it a directional cassette. Since virtually all expression cassettes are directional in this sense, those skilled in the art can easily determine the orientation of a given expression cassette in relation to another element, for example, the first selection marker gene. In various embodiments, the first selection marker gene may be orientated in a 5′-3′ orientation and the expression cassette may be orientated in a 3′-5′ orientation, whereby the 5′-3′ orientation may be the same direction as the origin of replication within the first circular DNA sequence.

In various embodiments, the expression cassette further comprises a selection marker gene, preferably a puromycin resistance gene. This selection marker gene may be termed as a second selection marker gene and is different to the first selection marker gene (i.e. hygromycin resistance gene) that is promoter-free. In various embodiments, the second selection marker gene is positioned upstream of the constitutive promoter. In various embodiments, the DNA sequence of interest comprises a first selection marker gene, and an expression cassette comprising a second fluorescent marker gene, and optionally a second selection marker gene, operably linked to a constitutive promoter. In various embodiments, the second fluorescent marker gene and second selection marker gene both operably linked to a constitutive promoter.

In various embodiments, the DNA sequence of interest comprises a first selection marker gene, an expression cassette comprising a second fluorescent marker gene, and optionally a second selection marker gene, operably linked to a constitutive promoter, and one or more additional genes. The one or more additional genes refer to genes that are “additional” to and different from the fluorescent marker gene and selection marker gene(s). In various embodiments, the one or more additional genes is positioned downstream of the first selection marker gene and upstream of the expression cassette.

These one or more additional genes may encode a protein or gene product that is desirably secreted from the eukaryotic host cells. In various embodiments, the one or more additional genes may include biosimilars and/or therapeutic genes that encode molecules that provide some therapeutic benefit, including proteins (e.g., secreted proteins, membrane-associated proteins (e.g., receptors), structural proteins, cytoplasmic proteins, and the like) functional RNAs (antisense, hammerhead ribozymes), and the like. Such genes include genes encoding an antibody.

The term “antibody”, as used herein, refers to a protein consisting of one or more polypeptide chains substantially encoded by all or part of the known immunoglobulin genes. Known immunoglobulin genes, for example in humans, include the kappa (κ), lambda (λ), and heavy chain genetic loci, which together comprise the multitude of variable region genes, and the constant region genes mu (μ), delta (δ), gamma (γ), epsilon (ε), and alpha (α) which encode the IgM, IgD, IgG (IgG1, IgG2, IgG3, and IgG4), IgE, and IgA (IgA1 and IgA2) isotypes respectively. The term “antibody”, as used herein is meant to include full length antibodies and antibody fragments, and may refer to a natural antibody from any organism, an engineered antibody, or an antibody generated recombinantly for experimental, therapeutic, or other purposes. The antibody fragments or variants referred to herein do however always include the heavy chain and light chain variable regions as disclosed herein. Accordingly, such fragments and variants include the known scFv fragments or scFv antibodies. The terms “antibody” and “immunoglobulin” are used interchangeably herein to relate to polypeptides encoded by immunoglobulin genes. The term “IgG” as used herein refers to a polypeptide belonging to the class of antibodies that are substantially encoded by a recognized immunoglobulin gamma gene.

In various embodiments, the antibody is an IgG antibody, for example an IgG1 antibody or an IgG1 kappa antibody. Such an antibody typically comprises two identical heavy chains and two identical light chains, both having the Ig domains detailed below. By “immunoglobulin (Ig) domain” herein is meant a region of an immunoglobulin that exists as a distinct structural entity as ascertained by one skilled in the art of protein structure. Ig domains typically have a characteristic β-sandwich folding topology. The known Ig domains in the IgG class of antibodies are VH, Cγ1, Cγ2, Cγ3, VL, and CL. VH and VL refer to the variable regions of the heavy (VH) and light (VL) chain and are herein defined by reference to their amino acid sequence. Cγ1, Cγ², and Cγ3 refer to the Ig domains of the constant part of the heavy chain, i.e. the domains more generally referred to as CH1, CH2 and CH3. The N-terminus of the CH1 or Cγ1 domain is linked to the C-terminus of the VH domain. CL relates to the constant part of the light chain and is linked to the C-terminus of the VL domain. The linkage is typically by a peptide bond. The full light chain thus comprises in N- to C-terminal orientation a VL and a CL domain. The full heavy chain thus comprises in N- to C-terminal orientation a VH, CH1, CH2 and CH3 domain. An IgG antibody comprises two full light chains and two full heavy chains.

In various embodiments, the DNA sequence of interest comprises one or more IgG genes, more preferably PD-1 genes, more preferably PD-1 genes encoding the PD-1 Heavy chain (HC) and PD-1 light chain (LC). More particularly, the DNA sequence of interest comprises monoclonal antibody PD-1 heavy and light chain genes.

In various embodiments, the DNA sequence of interest comprises two additional genes, wherein the two additional genes are orientated head-to-tail (CW), or head-to-head (CCW) relative to each other. In various embodiments, the DNA sequence of interest comprises monoclonal antibody IgG PD-1 heavy and light chain genes, wherein the two IgG PD-1 genes are orientated head-to-tail (CW) or head-to-head (CCW) relative to each other, with no other element between or separating the two PD-1 genes.

The term “head-to-tail” is used hereir to describe the orientation of two gene sequences in relation to each other, where the two gene sequences are positioned n a head-to-tail orientation when the 5′ end of the coding strand of one gene sequence is adjacent to the 3′ end of the coding strand of the other gene sequences, whereby the direction of transcription of each gene sequence proceeds in the same direction as that of the other gene sequence. The term “head-to-tail” may be abbreviated (5′)-to-(3′) and may also be indicated by the symbols (>>) or (5′>3′5′>3′). The term “head-to-head” as used herein describes the orientation of two gene sequences in relation to each other, whereby the two gene sequences are positioned in a head-to-head orientation when the 3′ end of the coding strand of one gene sequence is adjacent to the 3′ end of the coding strand of the other gene sequence, whereby the direction of transcription of each gene sequence proceeds towards the other gene sequence. The term “head-to-head” may be abbreviated (3′) to-(3′) and may also be indicated by the symbols (><) or (5>3′3′<5′)

The DNA sequence of interest may be designed for stable integration into a target genomic sequence of the eukaryotic host cell. The term “stably integrating a DNA sequence of interest into a target genomic DNA sequence of a eukaryotic host cell”, as used herein, refers to the stable integration of the DNA sequence of interest into the genomic landing pad of the eukaryotic host cell disclosed herein. The stably integrated DNA sequence of interest will thus be heritable to the progeny of a thus landing pad eukaryotic host cell. Said stable integration may be performed in vitro, ex vivo, or in vivo.

For λ-integrase-mediated insertion of the DNA of interest, genetically stable targeting of the genomic landing pads is carried out through the use of recombination partner sequences for lambda Integrase in conjunction with activities of λ-Integrase proteins and optionally co-factors inside eukaryotic host cells.

Accordingly, the methods for λ-integrase-mediated insertion disclosed herein comprise the step of providing a first circular DNA molecule comprising a lambda integrase recombination partner sequence of the lambda integrase recombination sequence comprised in the landing pad, and a DNA sequence of interest. That is, the lambda integrase recombination partner sequence of the first circular DNA molecule is compatible with, and capable of pairing with, the lambda integrase recombination sequence comprised in the genomic landing pad of the landing pad eukaryotic host cell or cell line disclosed herein.

In various embodiments, the lambda integrase recombination partner sequence comprised in the first circular DNA molecule is selected from wild-type attB sequence (SEQ ID NO:2) and/or wild-type attL sequence (SEQ ID NO:3) or derivatives thereof. The attB sequence has a 5′-3‘ BOB’ structure with B and B′ describing the left and right arms at the attB site and O describes the homologous core. The attL sequence is a hybrid of the attP and attB sequences.

In various embodiments, the first circular DNA molecule comprises the lambda integrase recombination partner sequence attB or attL, or both attB and attL or derivatives thereof.

In various embodiments, the lambda integrase recombination partner sequence of attP is positioned upstream of the DNA sequence of interest in the first circular DNA molecule.

In various embodiments, the DNA sequence of interest may also comprise at least one additional recombination site (e.g., loxP or FRT site) that is compatible with Cre and Flp recombinases.

Accordingly, in various embodiments, the DNA sequence of interest may comprise a loxP and/or a FRT site for Cre and/or Flp recombinases. In particular, in various embodiments where the DNA sequence of interest comprises one or more transgenes, the loxP and/or FRT site may flank the one or more transgenes, such that Cre and Flp recombinases can then excise undesirable DNA segments (other than the one or more transgenes) following transgene integration. In various embodiments, the FRT sequence is upstream of the one or more transgenes and the loxP sequence is downstream o the one or more transgenes.

The first circular DNA molecule may be a plasmid, vector, cosmid, bacterial artificial chromosome (BAC), bacteriophage, viral vector or hybrids thereof. In various embodiments, the first circular DNA molecule is a vector, which may be termed as a target vector.

“Vectors” are understood for purposes herein as elements—made up of nucleic acids—that contain a nucleic acid contemplated herein as a characterizing nucleic acid region. They enable said nucleic acid to be established as a stable genetic element in a species or a cell line over multiple generations or cell divisions. In particular when used in bacteria, vectors are special plasmids, i.e. circular genetic elements. Included among the vectors are, for example, those whose origins are bacterial plasmids, viruses, or bacteriophages, or predominantly synthetic vectors or plasmids having elements of widely differing derivations. Using the further genetic elements present in each case, vectors are capable of establishing themselves as stable units in the relevant host cells over multiple generations. They can be present extrachromosomally as separate units, or can be integrated into a chromosome resp. into chromosomal DNA. The term “target vectors” as used herein refer to vectors carrying the DNA sequence of interest and lambda integrase recombination partner sequence that targets the genomic landing pad for recombination.

Expression vectors encompass nucleic acid sequences which are capable of replicating in the host cells. In various embodiments, the vectors described herein thus also contain regulatory elements that control expression of the nucleic acids. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. In the present case at least one promoter for expression of a nucleic acid as contemplated herein is made available and used for expression thereof. Expression vectors can furthermore be regulated, for example by way of a change in culture conditions or when the host cells containing them reach a specific cell density, or by the addition of specific substances, in particular activators of gene expression. The expression vector may be based on plasmids well known to person skilled in the art such as pBR322, puC16, pBluescript® and the like. Thus, the expression vector may be termed as an expression plasmid.

The methods for λ-integrase-mediated insertion disclosed herein further comprises the step of providing a second circular DNA molecule comprising a nucleotide sequence encoding a lambda integrase or a functional variant or fragment thereof.

The term “lambda integrase” as used herein refers to any phage lambda-derived integrase that possesses site-specific recombination activities. As known in the art, the phage lambda integrase belongs, like Cre and Flp, to the tyrosine integrase family of the sequence-specific conservative DNA recombinases and catalyses the integrative recombination between two different recombination att sites.

In various embodiments, the integrase used in the method disclosed herein is a specific mutant of lambda integrase known in the art, namely the one disclosed WO2016022075A1, which is hereby incorporated by reference, and termed “IntC3”. Said IntC3 mutant integrase has the amino acid sequence set forth in SEQ ID NO:6.

Accordingly, the second circular DNA construct may comprise a nucleotide sequence encoding a lambda integrase having the amino acid sequence set forth in SEQ ID NO:6 or a functional variant or fragment thereof.

The nucleotide sequence can be DNA molecules or RNA molecules. They can exist as an individual strand, as an individual strand complementary to said individual strand, or as a double strand. With DNA molecules in particular, the sequences of both complementary strands in all three possible reading frames are to be considered in each case. Also to be considered is the fact that different codons, i.e. base triplets, can code for the same amino acids, so that a specific amino acid sequence can be coded by multiple different nucleic acids. As a result of this degeneracy of the genetic code, all nucleic acid sequences that can encode one of the above-described amino acid sequence are included in this subject of the invention. The skilled artisan is capable of unequivocally determining these nucleic acid sequences, since despite the degeneracy of the genetic code, defined amino acids are to be associated with individual codons. The skilled artisan can therefore, proceeding from an amino acid sequence, readily ascertain nucleic acids coding for that amino acid sequence. In addition, in the context of nucleic acids sequences, one or more codons can be replaced by synonymous codons. For example, every organism, e.g. a host cell of a production strain, possesses a specific codon usage. “Codon usage” is understood as the translation of the genetic code into amino acids by the respective organism. Bottlenecks in protein biosynthesis can occur if the codons located on the nucleic acid are confronted, in the organism, with a comparatively small number of loaded tRNA molecules. Also, its codes for the same amino acid, the result is that a codon becomes translated in the organism less efficiently than a synonymous codon that codes for the same amino acid. Because of the presence of a larger number of tRNA molecules for the synonymous codon, the latter can be translated more efficiently in the organism.

By way of methods commonly known today such as, for example, chemical synthesis or the polymerase chain reaction (PCR) in combination with standard methods of molecular biology or protein chemistry, a skilled artisan has the ability to manufacture, on the basis of known DNA sequences and/or amino acid sequences, the corresponding nucleic acids all the way to complete genes. Such methods are known, for example, from Sambrook, J., Fritsch, E. F., and Maniatis, T, 2001, Molecular cloning: a laboratory manual, 3rd edition, Cold Spring Laboratory Press.

The term “functional variant”, as used herein in relation to the integrase, relates to integrases that differ from the amino acid sequence set forth in SEQ ID NO:6 by one or more amino acid substitutions, additions or deletions but retain the functionality of the reference sequence. In such variants the amino acid positions that define the reference integrase C3, namely the positions 43F, 319G, and 336V may be invariable. The term also encompasses variants that comprise the sequence set forth in SEQ ID NO:6 but comprise N- and/or C-terminal extensions of 1 or more amino acids. Generally, the term “variant” covers such integrases that have at least 80%, or at least 90% sequence identity with the sequence set forth in SEQ ID NO:6 over their entire length, preferably at least 80, 81, 82, 83, 84, 85, 86, 87, 88, 89, 90, 91, 92, 93, 94, 95, 96, 97, 98, or 99% sequence identity. In these variants, the positions 43F, 319G, and 336V may still be invariable. The identity of nucleic acid sequences or amino acid sequences is generally determined by means of a sequence comparison. This sequence comparison is based on the BLAST algorithm that is established in the existing art and commonly used (cf. e.g. Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

The term “functional fragment” or “fragment”, as used herein in relation to the integrase, relates to integrases that differ from the amino acid sequence set forth in SEQ ID NO:6 by a deletion of one or more amino acids from its C- and/or N-terminus. Said fragments preferably retain full functionality. In various embodiments, such fragment differs from the reference sequence and that they lack 1-20 amino acids from their N- and/or C-terminus, for example 1-15 amino acids or 1-10 amino acids or 1-5 amino acids.

In various embodiments, the expression of the lambda integrase described herein, may be constitutive or stringently controlled by an inducible expression system such as the Tet on/off system that is well-known in the art.

The lambda integrases described herein may be able to perform the recombination reaction with or without a co-factor, such as IHF. The addition of the co-factor gene, in particular scIHF2, can have beneficial effects in that it may substantially reduce the lag phase in cultivation and thus can shorten incubation times needed to reach the desired cell density. As such, in various embodiments, the second circular DNA construct further comprises a nucleotide sequence encoding for an integration host factor (IHF), preferably single chain integration host factor 2 (scIHF2). In various embodiments, the lambda integrase and co-factor (e.g. IHF) are comprised in an expression cassette that is stably integrated into the genome of the host cell. In such embodiments, the expression of both, the lambda integrase and co-factor, may be stringently controlled by the same inducible expression control sequence.

In various embodiment, the second circular DNA construct comprises a nucleotide sequence encoding the lambda integrase, preferably IntC3, and an IHF, preferably scIHF2.

In various embodiments, the second circular DNA molecule is a plasmid, more particularly an expression plasmid.

The method for λ-integrase-mediated insertion further comprises the step of co-transfecting the landing pad eukaryotic host cell or cell line disclosed herein with the first circular DNA molecule and the second circular DNA molecule at suitable conditions to induce said co-transfection. The co-transfection leads to the presence of both, the first and second DNA molecules in the eukaryotic host cell(s).

Methods to co-transfect eukaryotic host cells are well known in the art and can be routinely applied by those skilled in the art. Transfection conditions, reagents and mediums can be selected by those skilled in the art based on the host organism used by resorting to general knowledge and techniques known in the art.

The method for λ-integrase-mediated insertion further comprises the step of inducing the expression of the lambda integrase to facilitate recombination and stable integration of the first circular DNA molecule into the landing pad of the host cell. Said induction can be done by adding an agent or compound that induces the expression control sequence for the integrase coding sequence.

The expression of the lambda integrase leads to the presence of both, the integrase and the first circular DNA molecule in the eukaryotic host cell. Their presence facilitates recombination of the two lambda integrase recombination sequences in the genomic landing pad and the first circular DNA molecule. Said recombination event results in the integration of the first circular DNA molecule into the genomic landing pad of the eukaryotic host cell leading to expression of the second fluorescent marker gene, and the cessation of the expression of the first fluorescent marker gene.

Moreover, the recombination event results in the creation of two genomic recombination junction sequences flanking the inserted DNA sequence of interest, wherein the two genomic recombination junction sequences are termed the right and left genomic recombination junction sequences. In various embodiments, following recombination and insertion, the first fluorescent marker gene is downstream of the right genomic recombination junction sequence, and the RNA polymerase promoter is upstream of the left genomic recombination junction sequence. Essentially, the RNA polymerase promoter is at or adjacent to the left genomic recombination junction sequence, and the first fluorescent marker gene is at or adjacent to the right genomic recombination junction sequence.

In various embodiments, the two genomic recombination junction sequences flanking the inserted DNA sequence of interest may be attR and attL sequences, where the two lambda integrase recombination partner sequences are attP and attB sequences, so that after site-specific recombination the integrated DNA sequence of interest is flanked by POB′ (attR) and BOP′ (attL) sequences. In various embodiments, the two genomic recombination junction sequences flanking the inserted DNA sequence of interest may be attP and attL sequences, where the two lambda integrase recombination partner sequences are attP and attL sequences.

Accordingly, intermolecular recombination occurs between the two lambda integrase recombination sequences (i.e. recognition sites) on different molecules, namely the genomic landing pad and the first circular DNA molecule, which leads to the fusion of the genomic landing pad with the first circular DNA molecule, more particularly the DNA sequence of interest.

By means of the intermolecular recombination between the two lambda integrase recognition partner sequences, as mediated by the lambda integrase, a eukaryotic host cell or cell line is obtained comprising the genomic integration of the first circular DNA molecule within the genomic landing pad.

Without wishing to be bound to any particular theory, it is believed that the relative concentrations of the first circular DNA molecule and the lambda integrase as well as the various parameters of the reaction condition can be optimized by routine experimentation to favour intermolecular recombination over intramolecular recombination.

This intermolecular recombination results in the RNA polymerase promoter of the landing pad being positioned upstream of the genomic recombination left junction sequence, and the first fluorescent marker gene being positioned downstream of the genomic recombination right junction sequence, causing the first fluorescent marker gene to no longer being operably linked to a promoter sequence and its expression is ceased. Hence, successful intermolecular recombination between the lambda integrase recombination partner sequences (e.g. attP and attB and/or attL) will generate eukaryotic host cells expressing the second fluorescent marker gene but not the first fluorescent marker gene.

In this regard, following intermolecular recombination, the second fluorescent marker gene represents a positive fluorescent marker gene, whereby the gain of expression of the second fluorescent marker gene that was not present in the parent eukaryotic host cell identifies a successful recombination event and product. Similarly, the first fluorescent marker gene represents a negative fluorescent marker gene (i.e. negative selection), whereby the loss or lack of expression of the fluorescent marker gene that was present in the parent eukaryotic host cell identifies a successful recombination event and product.

Accordingly, the method further comprises the step of identifying eukaryotic host cells comprising genomic landing pads into which the first circular DNA construct has stably integrated based on the detectable expression of the second fluorescent marker gene, and the absence of the detectable expression of the first fluorescent marker gene. The expression of the fluorescent marker genes may be detected by any conventional means known in the art. In various embodiments, the expression levels of the first and/or second fluorescent marker gene may be carried out by using flow cytometry.

The identifying step may further comprise culturing the transfected eukaryotic host cells under conditions selective for the selection marker gene comprised in the DNA sequence of interest. This ensures that only those cells that have been successfully transfected and DNA sequences of interest integrated into the genomic landing pad are grown. In particular, the transfected eukaryotic host cells may be cultured in a selection medium under conditions that allow the growth of eukaryotic host cells expressing the selection marker gene. As such, in various embodiments, the method further comprises the step of identifying eukaryotic host cells comprising genomic landing pads into which the first circular DNA construct has stably integrated based on the detectable expression of one or more selection marker genes and the second fluorescent marker gene, and the absence of the detectable expression of the first fluorescent marker gene.

Accordingly, there is provided a method of λ-integrase-mediated insertion of a DNA sequence of interest into a landing pad eukaryotic host cell, the method comprising:
(i) providing a first circular DNA molecule comprising a lambda integrase recombination partner sequence of attP and a DNA sequence of interest comprising a selection marker gene and a second fluorescent marker gene operably linked to a constitutive promoter;
(ii) providing a second circular DNA molecule comprising a nucleotide sequence encoding a lambda integrase (IntC3) having the amino acid sequence set forth in SEQ ID NO:6 or a functional variant or fragment thereof;
(iii) co-transfecting a eukaryotic host cell or cell line comprising a genomic landing pad described herein, with the first circular DNA molecule of (i) and the second circular DNA molecule of (ii) at suitable conditions to induce said co-transfection; and
(iv) inducing the expression of the lambda integrase to facilitate recombination and stable integration of the first circular DNA construct into the genomic landing pad of the eukaryotic host cell;
(v) identifying eukaryotic host cells comprising genomic landing pads into which the first circular DNA construct has stably integrated based on the detectable expression of the selection marker gene and second fluorescent marker gene, and the absence of the detectable expression of the first fluorescent marker gene.

After step (v), eukaryotic host cells identified as expressing the selection marker gene (e.g. hygromycin resistance) and the second fluorescent marker gene (e.g. eGFP) while not expressing the first fluorescent marker gene (e.g. mCherry), may be isolated, expanded and screened for confirming and verifying the stable genomic integration of the first circular DNA construct into the landing pad using specific PCR primer combinations and PCR sequence analysis based on the lambda integrase recombination sequences (i.e. att site pair) utilized as mentioned above. In particular, each primer pair used may be designed with respect to the left junction and the right junction with the resulting amplified products being sequenced to confirm the site of insertion.

The isolating of the eukaryotic host cells may comprise serial dilution to obtain single cell colonies that may be expanded to generate a homogenous population of cells, more particularly a monoclonal cell line, comprising the λ-integrase-mediated insertion of the DNA of interest. The single cell colonies may be picked and grown in 24-well culture plates and then expanded to 6-well plates. Genornc DNA may then be isolated frorn one or more of these cel: clones and checked for attP site integration into the genormic landing pad.

Accordingly, in various embodiments, the method of λ-integrase-mediated insertion further comprises isolating and digesting the genomic DNA of the eukaryotic host cell with one or more restriction enzymes and the digested products analysed by one or more conventional methods such as PCR, Southern blotting, electrophoresis and sequence analysis or a combinations thereof.

In various embodiments, the genomic DNA of the isolated and expanded eukaryotic host cell may be purified and digested by means of an endonuclease activity, preferably by means of one or more restriction enzymes. The choice of the endonuclease and the isolation method is within the knowledge of the person of average skill in the art.

In various embodiments, the restriction enzymes, may include, but are not limited to Nhel, HindIII, BgrGl, Pvul, SgrAl, Bsml, Bmtl, Agel, EcoRI and Mfel.

In various embodiments, the digested genomic DNA of the eukaryotic host cell may be analysed by using using specific PCR primer combinations and PCR sequence analysis based on the lambda integrase recombination sequences (i.e. att site pair) utilized as mentioned above. In particular, each primer pair used may be designed with respect to the left junction and the right junction with the resulting amplified products being sequenced to confirm the site of insertion, as well as the size of the insert. The PCR may comprise a combination of inverse and nested PCR, whereby PCR amplified products may be resolved by electrophoresis on agarose gels and amplified bands sequenced.

In various embodiments, the digested genomic DNA of the eukaryotic host cell may be analysed by Southern blotting. The Scuthern blot analysis may confirm a single copy integration of the DNA sequence of interest, within the landing pad. In particular, the digested genomic DNA of the eukaryotic host cell may be incubated with a probe for both the first and second fluorescent marker genes, and/or the DNA sequence of interest. As will be appreciated, suitable probes may be designed and prepared using well-known techniques and the knowledge of the skilled person. For example, an mCherry probe (first fluorescent marker gene) may use mCherry probe fwd and rev primers, an eGFP probe (second fluorescent marker gene) may use a eGFP probe fwd and rev primers, and an anti-PD1 IgG light chain probe (transgene probe) may use a PD1 LC probe fwd and rev primers. The probe-target hybrids on the Southern blots may be detected by a chemiluminescent assay.

As shown in the following working examples, the functionality of transgenic eukaryotic host cells generated was determined by the expression o IgG transgenes and secretion of functional monoclonal IgG, whereby a GMP-grade homologous Exp 293F cell line can be generated which may have potential use as transplantable mini bioreactors that represent safe and cost-effective alternatives to current clinical practice.

Further, as a proof-of-concept, a broad utility with expression constructs for anti PD-1 monoclonal antibodies was demonstrated which showed that the orientation of heavy and light chain transcription units profoundly affected antibody expression levels. Large transgenic vectors carrying heavy and light chain anti PD-1 monoclonal antibody transgenes in different orientations with respect to each other were inserted, thus permitting direct comparisons of PD-1 antibody expression yields from otherwise isogenic cells. The PD-1 protein is present on the surface of T cells and binds to the PD-1 ligand (PD-L1) expressed on cancer cells resulting in the inhibition of cancer cell killing by the immune cells. Monoclonal anti-PD-1 antibodies impede this interaction by binding to PD-1 as a promising novel anti-cancer strategy.

Accordingly, in various embodiments, the DNA sequence of interest may comprise PD-1 genes, more preferably IgG PD-1 genes encoding the PD-1 Heavy chain (HC) and PD-1 light chain (LC). In various embodiments, the DNA sequence of interest comprises two PD-1 genes, wherein the two PD-1 genes are orientated head-to-tail (CW) or head-to-head (CCW) relative to each other.

For downstream applications, it is important that the eukaryotic cells harbouring transgenes remain stable over long periods in the absence of any selection pressures.

Importantly, transgene instability and variation in expression were not observed in the absence of selection pressure, thus enabling reliable long-term biotherapeutics testing or production. Therefore, the landing pads disclosed herein for lambda integrase can be targeted with multi-transgene constructs and offers future modularity involving additional genome manipulation tools to generate sequential or nearly seamless insertions.

Accordingly, in various embodiments, the expression of the DNA sequence of interest, more preferably the one or more additional genes, is stable and sustained for at least 5, 6, 7, 8, 9, 10, 11, 12, 13 or 14 days in the absence of selection pressure. In various embodiments, the expression of genes within the DNA sequence of interest, is stable and sustained for at least 14 days in the absence of selection pressure.

In contrast to existing methods, the methods disclosed herein attain the capability of generating novel safe harbor sites in the genome of eukaryotic cells for artificial gene docking sequences without the application of selection pressure. This allows for the desired sustained and uniform transgene expression after delivery of said transgenes (DNA sequence of interest) by lambda-Integrase-mediated recombination from a single genomic locus. Other advantages may include the technical ease and cost effectiveness of the A-Integrase genome insertion platform and the potential inclusion of seamless vectors. Furthermore, unlike existing methods developed mainly for CHO cells, the present invention may be tailored for use in human cells with a broad scope of applications.

In addition, it has been demonstrated that the exemplified PD-1 expressing cells can be encapsuled into bio-compatible mini-bioreactors and continue to secrete antibodies, thus providing a basis for future cell-based applications for more effective and affordable therapies. Accordingly, the methods disclosed herein were shown to generate PD-1 antibody-expressing cells capable of being encapsulated to create cellulose-based mini bioreactors producing PD-1 antibodies for possible future allogeneic cell-based therapies.

Accordingly, in various embodiments, the method further comprises encapsulating the eukaryotic host cell obtained from step (v), preferably using a cellulose sulfate-based encapsulation protocol. Gels or capsules used for said encapsulating or embedding of cells may be made of either biological or synthetic polymers. Examples of gels or capsules used for such purposes include Matrigel (manufactured by Corning), PuraMatrix (manufactured by 3D Matrix), VitroGel 3D (manufactured by The Well Bioscience), collagen gel (manufactured by Nitta Gelatin Co., Ltd.), and alginate gel. (manufactured by PG Research Co., Ltd.), Cell-in-a-Box (manufactured by Austrianova), and the like, which are commercially available as hydrogel or embedding culture kits for cell culture can be used.

The eukaryotic host cell and cell line containing the genomic landing pad with an integrated DNA sequence of interest may be referred to herein as “transgenic eukaryotic host cell or cell line”.

Accordingly, there is also provided a transgenic eukaryotic host cell or cell line, preferably a monoclonal cell line, obtained by the methods disclosed herein.

All embodiments disclosed above in relation to the methods disclosed herein for λ-integrase-mediated insertion of a DNA sequence of interest, similarly apply to the transgenic eukaryotic host cell disclosed herein or transgenic eukaryotic host cell line obtained therefrom, and vice versa.

These cells may then be encapsulated and used as transplantable mini bioreactors. Such uses thus also form part of the present invention.

The present invention is further illustrated by the following examples. However, it should be understood, that the invention is not limited to the exemplified embodiments.

### WORKING EXAMPLES

### Materials and Methods

Expi293F cell culture: Expi293F cells were cultured in suspension Expi293 Expression Medium (Gibco, Life technologies) with 100 Units/ml of Penicillin and Streptomycin (Gibco, Life technologies) in 125 ml flasks in an orbital shaker incubator at 125 rpm and 37° C. with >80% relative humidity and 8% CO₂. A cell density of 3 million cells per ml was maintained and the seeding cell density was 0.3 million cells per ml. These cells were adapted to adherent culture following a previously reported protocol [49]. In brief, 2 million cells from the suspension culture were plated in Dulbecco's Modified Eagle Medium (DMEM) supplemented with 10% foetal bovine serum (FBS), 1% L-glutamine and 100 Units/ml of Penicillin and Streptomycin (Gibco, Life Technologies) at 37° C. under 5% CO₂ in humidified conditions in a 10 cm tissue culture plate (TPP, Switzerland). For readaptation of cells from adherent to suspension culture, cells were detached using Trypsin-EDTA (Gibco, Life Technologies) from a confluent 150 cm² tissue culture flask (TPP, Switzerland), and 15 million cells were suspended in 25 ml Expi293 Expression Media and placed in a 125 ml flask in an orbital shaker incubator. For selection of recombinants, hygromycin B (Invitrogen, Life Technologies) was used at 500 μg/ml. After selection, pure clones were expanded and transferred to flasks containing Expi293F expression media to further readapt them to suspension culture as previously described [19].

Plasmids: Standard molecular cloning protocols were used to construct the plasmids used in this study. Q5® High-Fidelity DNA Polymerase (NEB) was used for PCR amplifications and E. coli DH5α was used for plasmid preparation. The construction of the Int expression vectors (pCMVssInt-h/218 and -Int-C3) has been described previously [20]. pEF_attP_mCherry was prepared by replacing the Neo_IRES_dTomato cassette with the mCherry_loxP cassette (PCR amplified using mCherry_BamHI and SV40_LoxPmCherry_HindIII primers listed in Table 1) in pEF_attP_Neo_IRES_dTomato between the BamHI and HindIII sites.

The pattB_HygroR_eGFP plasmid was constructed by cloning the eGFP expression cassette into the Nael site in pattB_HygroR by homologous recombination cloning using the ClonExpress Ultra One Step Cloning Kit (Vazyme, Nanjing, China) using the manufacturer's protocol. The pattB_HygroR_PD1 HC and LC_PuroR_eGFP plasmids were constructed and obtained from e-Zyvec (Loos, France).

Transfections: For suspension cultures, 5×10⁵ Expi293F cells were plated in 2 mIs of Expi293 Expression Medium per well of a 6-well plate (TPP, Switzerland). Cells were transfected with lug of pEF_attP_mCherry using Lipofectamine 2000 (Invitrogen, Life technologies) using DNA: Lipofectamine ratios of 1 μg: 3 μl. For each transfection per well, complexes were prepared by mixing DNA and Lipofectamine reagent separately diluted in 100 μl of Opti-MEM medium (Life technologies) and incubating for 20 min at room temperature. The transfection mix was then added drop wise onto the cells and incubated overnight. The next day, the medium was changed after centrifugation at 400 g for 4 minutes. After 2 days, cells were suspended in 20 ml Expi293 Expression Media and transferred to 125 ml suspension culture flasks.

For transfection of adherent Expi293F cells, 3×10⁵ cells were seeded in 2 mIs DMEM growth medium with 10% FBS per well of a 6-well plate (TPP, Switzerland) a day before transfection to obtain 70-90% confluence at the time of transfection. Transfections for targeting in adherent cells were carried out with Lipofectamine 2000 or 3000 (Invitrogen, Life technologies) using DNA: Lipofectamine ratios of 1 μg: 3 μl as described above, under antibiotics free DMEM growth medium with 10% FBS and transfection was allowed to proceed for 4-6 h before replacing with fresh growth medium.

Antibiotic selection and screening for targeted cell clones in adherent culture: Forty-eight hours post transfection in adherent cells, selection with Hygromycin B in growth medium at 500 μg/ml (and for plasmids carrying the IgG genes also Puromycin at 1 μg/ml) was initiated. The selection medium was replaced every two days until colonies formed. At this stage, colonies were picked by carefully scraping patches of cells with a pipette tip and transferred to 24-well tissue culture plates for clonal expansion. The clones were sequentially expanded from 24-well to 6-well tissue culture plates. Genomic DNA was extracted using the DNeasy Blood & Tissue Kit (Qiagen, GmbH) as per the manufacturer's protocol. Clones were further maintained without antibiotic selection in the culture media.

Clonal cell lines generation: Clonal cell lines were generated by serial dilution of the cells in adherent culture. For suspension cell culture (mCherry⁺bulk population), cells were first adapted to adherent culture and then serially diluted in the 96-well plates. In the first well, 2000 cells were added in 200 μl DMEM growth media with 10% FBS and 100 μl media was added in other 7 wells of the column. Cells were serially diluted in 1:2 dilution till the 8^{th} well by transferring 100 μl media from the first well to the second well and so on. Next 50 μl media was added in the remaining wells of the plate and cells from the 8 wells of first column were transferred to the corresponding 8 wells of the next column by transferring 50 μl media. The process was repeated till the last column. Later, 50 μl media was added in each well and cells were maintained for 3 to 5 days until single colonies appeared. The selected colonies were further subjected to the same process again to get a homogenous population. For targeted cells, same process was repeated with the picked colonies.

Identification of recombination events by PCR screening: PCR of DNA extracted from the clones (adherent or suspension cultures) was performed using GoTaq Flexi DNA polymerase (Promega) to amplify genomic recombination junctions using the primers listed in the figure descriptions and 500 ng of genomic DNA from each recombinant clone or parental cells as a template in 50 μl reactions. The thermal cycling parameters for the PCR was as follows: initial denaturation at 95° C. for 2 min, 35 cycles of denaturation at 95° C. for 1 min, annealing at 55° C. for 1 min and extension at 72° C. for 1 min per kb, and a final step of 72° C. for 5 min. The PCR products were analysed by electrophoresis in 0.8% agarose (Seakem Agarose, Lonza, USA) gels in 0.5×TBE (Tris-Boric acid-EDTA buffer) containing 0.5 μg/ml ethidium bromide. PCR-generated products were compared with DNA standard markers and digitally documented under UV illumination (Quantum Vilber Lourmat, Germany). PCR-amplified products were analysed by sequencing.

Flow cytometry: A FACS Calibur Flow Cytometer (Becton Dickson) and CELL Quest software (Becton Dickson) were used to quantify mCherry+ and eGFP+ cells. Cells were harvested and suspended in the corresponding media. A dot plot of side scatter (SSC) versus forward scatter (FSC) was used to gate live cells to separate them from aggregated and dead cells. For further analysis, mCherry versus FSC and mCherry versus eGFP plots were constructed for gated cells. Data was analysed using BD FACSDiva™ software, and mCherry-/mCherry+ and eGFP−/eGFP+ cells for each sample were indicated (as %) in each quadrant.

Southern blot analysis: Southern blot probes were prepared using the PCR DIG Probe Synthesis Kit (Roche) as per manufacturer's protocol. For the mCherry probe, mCherry_probe_fwd and 116_mCherry_rev primers were used, for the eGFP probe, 241_eGFP_probe_fwd and 242_eGFP_probe_rev primers were used and for the anti-PD1 IgG light chain probe, PD1 LC_probe_fwd and PD1 LC_probe_rev primers were used.

Genomic DNA was purified using the DNeasy Blood & Tissue Kit (Qiagen, GmbH). 20 μg of genomic DNA was subjected to restriction digestion using 50 U of the respective enzyme in 200 μl overnight at 37° C. DNA was ethanol precipitated and dissolved in 20 μl TE buffer (pH 8.0). Target vectors were linearized with single cutter restriction enzymes and diluted to 10⁷, 10⁸ copies per μl. Digested genomic DNA samples were resolved overnight on a 0.8% agarose gel in 1×TAE (Tris-acetate-boric acid) buffer, with 1 kb DNA ladder (Thermo Scientific) and 1 μl of positive control samples. After transfer to the positively charged nylon membrane (Roche) by capillary transfer, Southern blotting employing the respective probes as indicated, was performed using the DIG-High Prime DNA Labelling and Detection Starter Kit II (Roche) as per the manufacturers' protocol. The probe-target hybrids on the blots were detected by chemiluminescent assay using ChemiDoc MP (Biorad).

Inverse PCR: Inverse PCR and subsequent nested PCRs were performed using GoTaq Flexi DNA polymerase (Promega).

Genomic DNA was purified from isolated clones and 2 μg genomic DNA was digested with the restriction enzymes Nhel, HindIII, Pvul, SgrAl, Bsml, Bmtl, Agel, EcoRI and Mfel overnight. The digested genomic DNA was purified by PCR purification kit (Qiagen). T4 DNA ligase (NEB) was used to self-ligate 250 ng of digested genomic DNA using 1 μl of enzyme in a 250 ul reaction volume to promote self-ligation with overnight incubation. Next day, ligated DNA was again purified using the PCR purification kit (Qiagen) and inverse PCR was performed with eluted DNA as follows: initial denaturation at 95° C. for 2 min, 35 cycles of denaturation at 95° C. for 1 min, annealing at 55° C. for 1 min and extension at 72° C. for 8 min, and a final step of 72° C. for 10 min using the primers EF_rev_474 and 67_mCherry_fwd listed in Table 1. Next, nested PCR was performed with 2 μl of inverse PCR product in a 50 μl reaction using same conditions with the EF_rev_104 and mCherry_fwd_597 primers listed in Table 1. PCR products were resolved on 0.8% agarose gels and amplified bands were sequenced.

IgG purification and SDS-PAGE: Cells from clones 6B1 and 23A4 were seeded at 0.5 million cells per ml density in 30 ml Expi293 Expression Medium in 125 ml culture flask and allowed to grow for five to seven days until dead cells appeared. IgG secreted from clone 6B1 and 23A4 was isolated from the culture media after pelleting the cells at 500×g for 5 minutes. The NAb™ Protein G Spin Kit (Thermo Scientific) was used following the manufacturer's protocol. In brief, collected media was incubated with resin overnight in a cold room. Media was removed by centrifugation the next day and the resin was washed with the provided buffer followed by elution in the given solution. IgG was eluted in different fractions and the IgG concentration was measured using a NanoDrop™ 2000/2000c Spectrophotometer (Thermo Scientific). 15 μl from each eluted sample was prepared with 4×loading dye and resolved in Invitrogen Bolt 4-12% Bis-Tris Plus precast gels (Thermo Fisher Scientific).

Cell Encapsulation: IgG secreting clone 6B1 cells were micro-encapsulated using the Cell-in-a-Box® kit from Austrianova Singapore following the manufacturer's protocol. In short, about 0.8 million cells were suspended and mixed well into 1 ml of a proprietary sodium cellulose sulphate solution (Solution 1), drawn up into a syringe and a fine, blunt-ended needle added. The cell/SCS mixture was dropped into a constantly stirring gelation bath made up of a second polymer (Solution 2) at the rate of 1-2 drops per second. More than 30 capsules were obtained and incubated in Solution 2 for 5 minutes with constant stirring in order to create a stable membrane around the capsule. This step was followed by three PBS washes and three culture media washes. The capsules were then transferred by serological pipette into culture dishes and placed in an incubator with fresh media. The porous nature of the membrane allowed the capsules to be cultured over several days whilst the cells divided inside until the capsules were full with thousands of cells.

IgG binding assays by ELISA: The amount of IgG secreted from encapsulated cells into media was estimated using a Human IgG ELISA kit (abcam) as per the manufacturer's manual. In brief, 100 μl media from different samples of encapsulated cells was added into the wells of a 95-well plate provided in the kit and incubated for 2.5 hours followed by several washing steps and incubation in biotinylated IgG solution for 1 hour. After further washing, plates were incubated with HRP-streptavidin solution for 45 minutes. This was followed by further washing and incubation with TMB substrate reagent for 30 minutes. A microplate reader was used to quantify emissions at 450 nm after adding stop solution. All incubations were performed at room temperature with constant shaking.

Biotinylated recombinant human PD-i ((Sino Biological #10377-H08H) was immobilized on the neutravidin (ThermoFisher scientific, #31000)-coated ELISA plate, to which the anti-PD-i antibodies at the indicated concentrations were added. Following incubation and washes, peroxidase-conjugated F(ab′)2 Fragment Goat Anti-Human IgG (JACKSON ImmunoResearch, #i109-036-098) at i1:3000 dilution was added to each well and the samples were incubated at RT for an hour. The wells were then washed and the substrate TMB (SurModics, #TMBW-i1000-0i) was added. The reactions were stopped by adding half volume of 1M HCl and the absorbance at 450 nm was measured on a microplate reader.

TABLE 1
List of nucleotide and amino acid sequences.

| SEQ ID NO: | Description | Sequence |
|---|---|---|
| 1 | Wild-type attP | TCTGTTACAGGTCACTAATACCATCTAAGTAGTTGATTCAT |
| | Source: | AGTGACTGCATATGTTGTGTTTTACAGTATTATGTAGTCTG |
| | bacteriophage | TTTTTTATGCAAAATCTAATTTAATATATTGATATTTATATCA |
| | lambda | TTTTACGTTTCTCGTTCAGCTTTTTTATACTAAGTTGGCATT |
| | | ATAAAAAAGCATTGCTTATCAATTTGTTGCAACGAACAGGT |
| | | CACTATCAGTCAAAATAAAATCATTATTTGATTTC |
| 2 | Wild-type attB | CTGCTTTTTTATACTAACTT |
| | Source: E.coli | |
| 3 | Wild-type attL | CTGCTTTTTTATACTAAGTTGGCATTATAAAAAAGCATTGC |
| | Source: E.coli | TTATCAATTTGTTGCAACGAACAGGTCACTATCAGTCAAAA |
| | | TAAAAATCATTATTTGATTTC |
| 4 | Wild-type attR | TCTGTTACAGGTCACTAATACCATCTAAGTAGTTGATTCAT |
| | Source: E.coli | AGTGACTGCATATGTTGTGTTTTACAGTATTATGTAGTCTG |
| | | TTTTTTATGCAAAATCTAATTTAATATATTGATATTTATATCA |
| | | TTTTACGTTTCTCGTTCAGCTTTTTTATACTAACTTG |
| 5 | Modified attP | TCTGTTACAGGTCACTAATACCATCTAAGTAGTTGATTCAT |
| | Bold indicates | AGTGACTGCATATCTTGTGTTTTACAGTATTAATTAGTCTG |
| | nucleotide | TTTTTTATCCAAAATCTAATTTAATATATTGATATTTATATCA |
| | substitutions from | TTTTACGTTTCTCGTTCAGCTTTTTTATACTAAGTTGGCATT |
| | the wild-type attP | ATAAAAAAGCATTGCTTATCAATTTGTTGCAACGAACAGGT |
| | Source: Synthetic | CACTATCAGTCAAAATACAATCATTATTTGATTTC |
| 6 | C3 lambda | MGRRRSHERRDLPPNLYIRNNGYYCYRDPRTGKEFGLGRD |
| | integrase mutant | RRFAITEAIQANIELFSGHKHKPLTARINSDNSVTLHSWLDRY |
| | (IntC3) | EKILASRGIKQKTLINYMSKIKAIRRGLPDAPLEDITTKEIAAML |
| | Source: Synthetic | NGYIDEGKAASAKLIRSTLSDAFREAIAEGHITTNHVAATRAA |
| | | KSKVRRSRLTADEYLKIYQAAESSPCWLRLAMELAWTGQRV |
| | | GDLCKMKWSDIVDGYLYVEQSKTGVKIAIPTALHIDALGISMK |
| | | ETLDKCKEILGGETIIASTRREPLSSGTVSRYFMRARKASGLS |
| | | FEGDPPTFHELRSLSARLYGKQISDKFAQHLLGHKSVTMAS |
| | | QYRDDRGREWDKIEIK |
| 7 | 39_EF_fwd | GTCCAGGCACCTCGATTAGTTCTC |
| 8 | 238_Hygro_rev | CGAAGCTGAAAGCACGAGATTCTTC |
| 9 | 201_ori_fwd | GATAAGTCGTGTCTTACCGGGTTG |
| 10 | 66_mCherry_rev | CTTGTAGATGAACTCGCCGTCCTG |
| 11 | mCherry_probe_fwd | ATGGTGAGCAAGGGCGAGGAGG |
| 12 | 116_mCherry_rev | GCTTCAAGTAGTCGGGGATGTCG |
| 13 | 241_eGFP_probe_fwd | CCGACCACATGAAGCAGCACG |
| 14 | 242_eGFP_probe_rev | GTTCTGCTGGTAGTGGTCGGCG |
| 15 | EF_rev_474 | AATGGCTAGAGACTTATCGAAAGCAGC |
| 16 | 69_mCherry_fwd | CGACATCCCCGACTACTTGAAGC |
| 17 | 67_mCherry_fwd | CGACATCCCCGACTACTTGAAGC |
| 18 | EF_rev_104 | TACACGACATCACTTTCCCAGTTTACCC |
| 19 | mCherry_fwd_597 | GTCAACATCAAGTTGGACATCACCTCC |
| 20 | C17_gnmc_fwd | GTAGTTTGGGTTTGTTTGTCTCCAGC |
| 21 | 255_pUC_ori_rev | CTTTCTCATAGCTCACGCTGTAGG |
| 22 | C17_gnmc_rev | GAAGTGAAAGGGAAGACAAGAACATGG |
| 23 | Amp_rev_498 | CGTCGTTTGGTATGGCTTCATTCAG |
| 24 | 235_Puro_rev | GAGGAAGAGTTCTTGCAGCTCGGTG |
| 25 | 231_Puro_rev | GGAACCGCTCAACTCGGCCATGC |
| 26 | PD1LC_fwd | TGACGGTACTGGGACAACCTAAAGC |
| 27 | PD1HC_fwd | GACTACGGGGCAGGTCCTTACTACT |
| 28 | PD1LC_probe_fwd | CCAAAGCGTCCTCACTCAACC |
| 29 | PD1LC_probe_rev | GTAACAGACGGAGCCGCTTTAGG |
| 30 | mCherry_BamHI | CTATGGATCCCCACCATGGTGAGCAAGGGCGAG |
| 31 | Sv40_LoxPmCherry_ | TGCCAAGCTTATAACTTCGTATAGCATACATTATACGAAGT |
| | HindIII | TATCCCGATCCAGACATGA |

### Results

### Example 1

Strategy for Insertion of Functional Single Copy Artificial Docking Sites for Sustained and Homogeneous Transgene Expression from the Human Genome

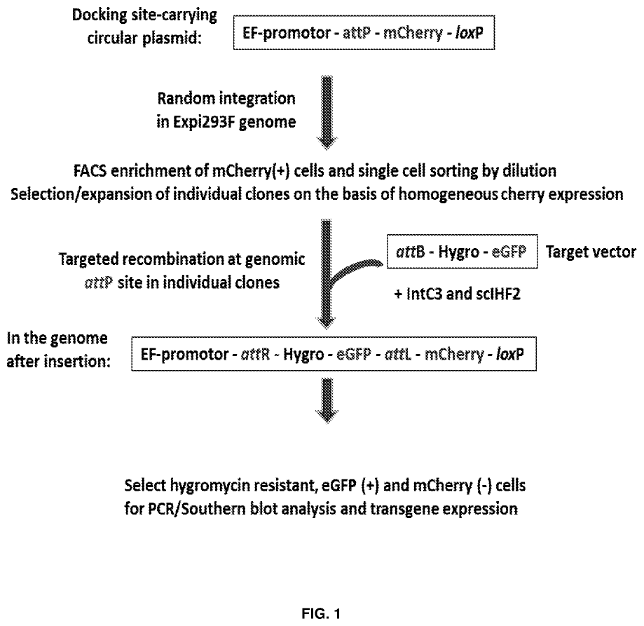

The following strategy was designed for the insertion of single copy landing pads for the A-Integrase genome insertion platform (FIG. 1): A bacterial plasmid that carries a eukaryotic RNA polymerase II (RNAP II) promoter (e.g. EF promoter) driving transcription of a fluorescent marker protein (e.g. mCherry) was generated by standard cloning procedures. The promoter and the marker gene flank the recombination sequence attP from the λ-Integrase-based recombination platform as described in PCT/DE00/02947, which is incorporated by its entirety herein. This attP sequence (SEQ ID NO:1, See Table 1) was altered to remove ATG start codons in its 5′-3′ (P—O—P′) sequence, which would otherwise interfere with the expression of the downstream coding region. Furthermore, one copy of a cognate sequence IoxP for the Cre recombinase was placed immediately downstream of the polyA signal of the mCherry gene. It will be appreciated that a recombination site (FRT) for the FLP recombinase may be included in the landing pad immediately upstream of the EF promoter.

This circular bacterial plasmid was introduced into human Expi293F cells by standard transfection methods. Transfected cells were cultured for two days, and fluorescent cells were sorted three times at seven days intervals. Stable fluorescent cells were maintained as a bulk culture. The cells were subsequently diluted to obtain single cells which gave rise to monoclonal cell lines. Monoclonal cell lines that visually maintained homogenous mCherry expression and were confirmed by PCR to harbor the entire landing pad were expanded and split into aliquots.

A fraction of cells was transfected with a circular target vector. An example of such target vector (p-attB-hygro-eGFP) contains the wild-type attB sequence and a downstream promoter-free resistance marker gene (e.g. hygromycin). An expression cassette for a second fluorescent marker gene (e.g. EGFP) was placed immediately downstream of the hygromycin gene. It will be appreciated that recombination sequences FRT and IoxP can be included in the design of target vectors. These additional SSR sequences can ultimately be used for the removal of unwanted sequences from the targeted genomic landing pad using standard techniques, such as transfection of messenger RNAs for the corresponding recombinases Cre and Flp.

The target vector was co-transfected with expression plasmids for the λ-Integrase Int-C3 and co-factor scIHF2 into the selected monoclonal mCherry(+) Expi293F lines. The recombinase Int-C3 will catalyze DNA strand exchange between the modified genomic attP sequence (in the landing pad) and the incoming, target vector-born attB site to insert the entire circular vector at the genomic landing pad. Correct recombination places the hygromycin coding region under the control of the genomic promoter within the landing pad, which can be selected for by exposing cells to hygromycin in the culture medium.

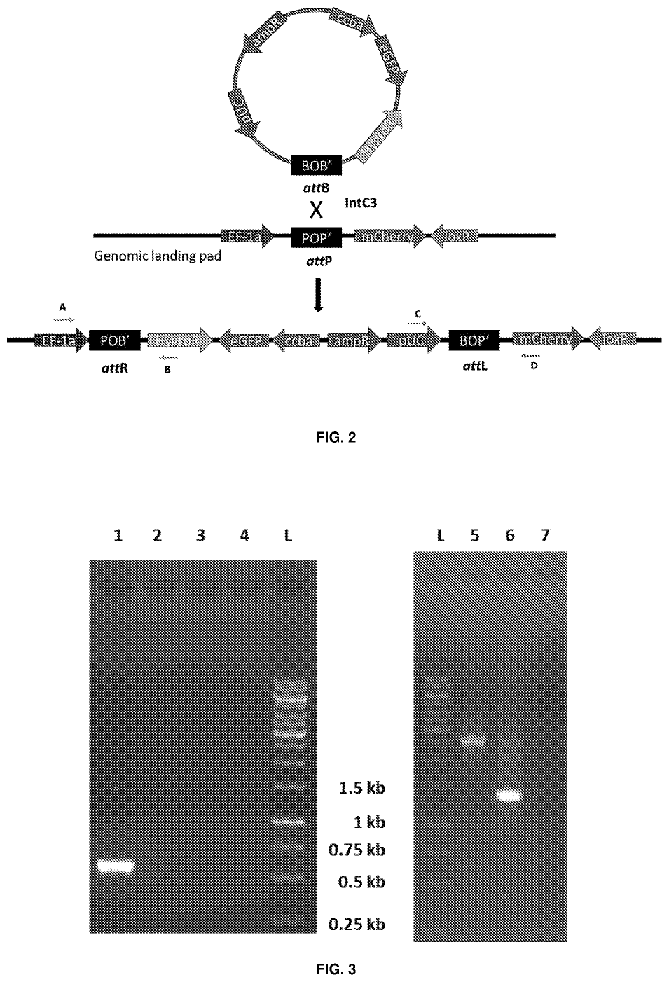

Furthermore, the desired insertion of the target vector will switch expression of fluorescent markers, e.g. here from mCherry(+) to EGFP(+), as depicted in FIG. 1 and FIG. 2. This switch can be used to isolate EGFP(+) and mCherry(−) cells. Successful landing pad targeting can subsequently be determined by genomic polymerase chain reaction (PCR) using primer pairs A&B and C&D which flank the resulting hybrid recombination junction attR and attL, respectively (FIG. 2).

### Verification of Successful Landing Pad Targeting in Bulk Cells by PCR

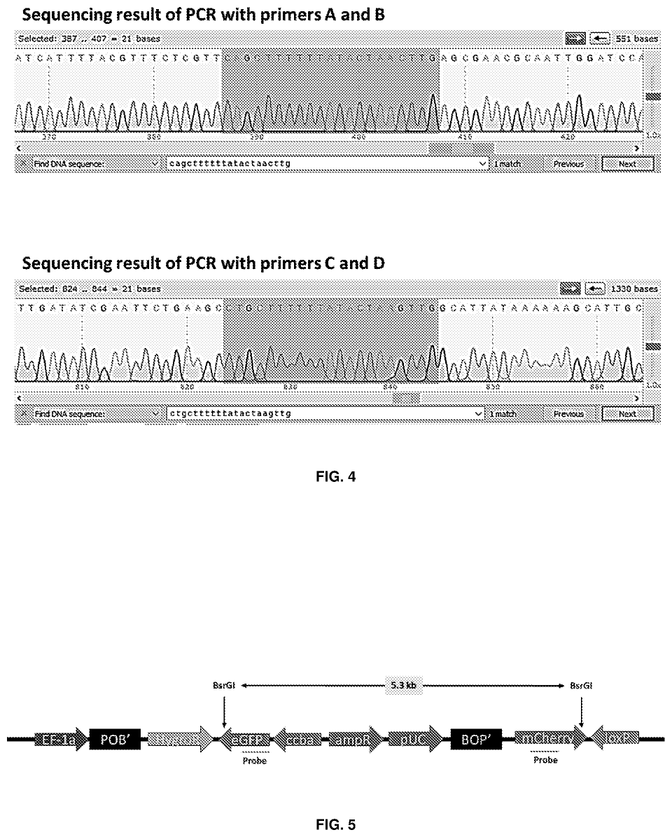

The monoclonal mCherry(+) Expi293F cell line #17, derived from the strategy outlined above, was transfected with the target vector p-attB-hygro-EGFP together with expression vectors for Int-C3 and scIHF2, followed by selection of successful targeting events with hygromycin. Surviving resistant cells (bulk, unsorted) were harvested, their genomic DNA isolated, and analyzed by PCR for the presence of both recombination junctions. Compared with parental #17 cells used as control, selected cells yielded the desired PCR products which were sequenced (FIG. 3). DNA sequencing confirmed that precise recombination between the genomic attP and attB on the target vector was mediated by Int-C3, and recombination resulted in two hybrid recombination junctions attR and attL (FIGS. 2 and 4).

### Verification of Successful Landing Pad Targeting in Monoclonal Cell Lines by PCR and Southern Blotting

Two monoclonal landing pad-containing mCherry(+) Expi293F cell lines, #17 and #29, were transfected with the target vector p-attB-hygro-EGFP followed by selection of targeting events with hygromycin as described. However, individual EGFP(+) or EGFP(−) monoclonal cell lines were established after hygromycin selection and genomic DNA isolated. FIG. 3 exemplifies results obtained with parental mCherry(+) line #17, where the chosen EGFP(+) cell line showed the expected PCR products for both junctions (lanes 2 and 9; arrows) like the positive controls using previously established bulk cultures (lanes 4 and 11). As expected, neither the EGFP(−) cell line (lanes 1 and 8) nor the negative control of untargeted clone #17 cells (lanes 3 and 10) yielded these PCR products.

In order to further verify successful recombineering into the artificial landing pad residing in the genome of mCherry(+) Expi293F cell lines #17 and #29, Southern blot (SB) analysis was performed with genomic DNA from respective monoclonal hygromycin resistant and EGFP(+) cell lines that were verified by PCR. This analysis included hygromycin-resistant yet EGFP(−) lines as well as the two parental mCherry(+) Expi293F lines as negative controls. Genomic DNA was digested with restriction enzyme BsrGI, which is predicted to cut DNA once in the target vector and once in the genomic landing pad, thus resulting in a 5.3 kb genomic DNA fragment. Probes complementary to the EGFP or mCherry gene were used on the target vector to identify DNA fragments (FIG. 5).

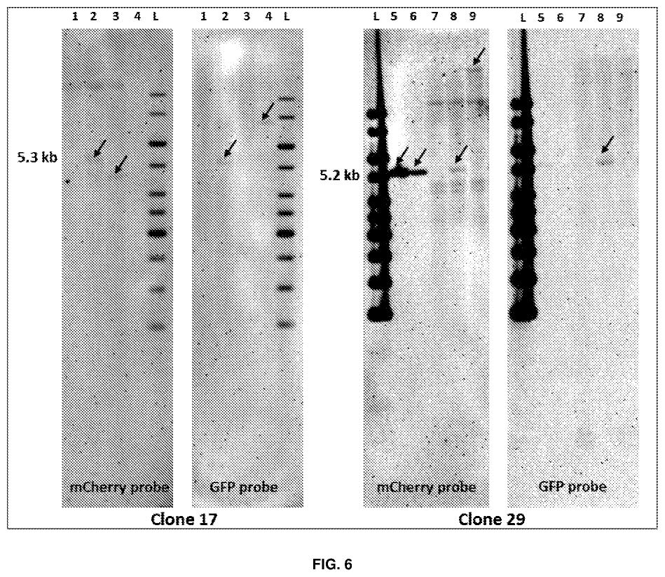

Using mCherry as a probe, the SB analysis revealed that each of the two monoclonal mCherry lines carry a single copy of the landing pad (FIG. 6; lanes 3 and 9) when compared to parental Expi293F cells as negative control (FIG. 6; lanes 1 and 7). Recombination between the target vector and genomic landing pad resulted in the expected 5.3 kb genomic fragment (FIG. 6; lanes 2 and 8). This conclusion was corroborated with the EGFP probe, which also generated a signal at 5.3 kb (FIG. 6; lanes 2 and 8). Controls for the functionality of the probes included linearized attB-hygro-EGFP target vector (FIG. 6; lane 4; 7.5 kb) and linearized landing pad-containing mCherry vector (FIG. 6; lanes 5 and 6; 5.2 kb). Combined, these data revealed that the two monoclonal mCherry(+) cell lines carry only a single copy of the landing pad and that Int-C3 can precisely insert a circular 7.2 kb target vector into the docking site.

Long-Term Homogeneous and Selection-Free Transgene Expression from Targeted Docking Sites

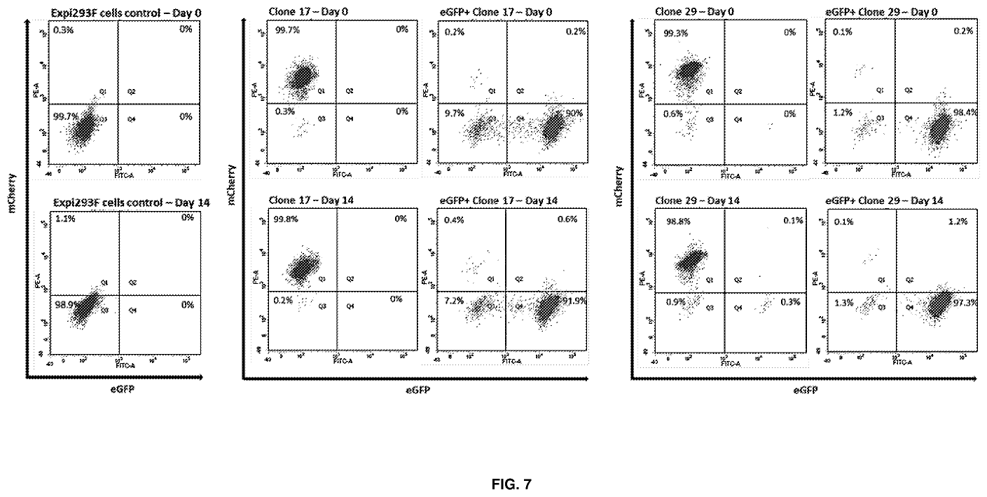

EGFP(+) clonal cell lines generated after p-attB-hygro-EGFP transfection in mCherry(+) Expi293F lines #17 and #29 and subsequent hygromycin selection were analyzed for their genetic stability and sustained transgene expression. In order to demonstrate this, EGFP(+) cell lines were maintained in hygromycin free media after confirmation of successful integration of p-attB-hygro-EGFP at the unique attP site. Cell lines were readapted to grow in suspension culture in Expi293 Expression media in a shaker incubator. Cells were maintained in suspension culture, and EGFP expressing cell populations were analyzed by flow cytometry at day 0 and day 14 (FIG. 7). Sustained expression of EGFP reporter was observed in both clones that remained constant for over two weeks during multiple cell passages. The percentage of EGFP expressing cells was >90% in both Expi293F targeted cell lines, as determined by flow cytometry. Furthermore, no significant change in the homogeneity was observed.

This data revealed that the transgene integration in the genome was stable and its expression was able to sustain for at least two weeks in the absence of selection pressure.

### Example 2

Generation of Expi293F Cell Lines with Single-Copy Artificial Docking Sites

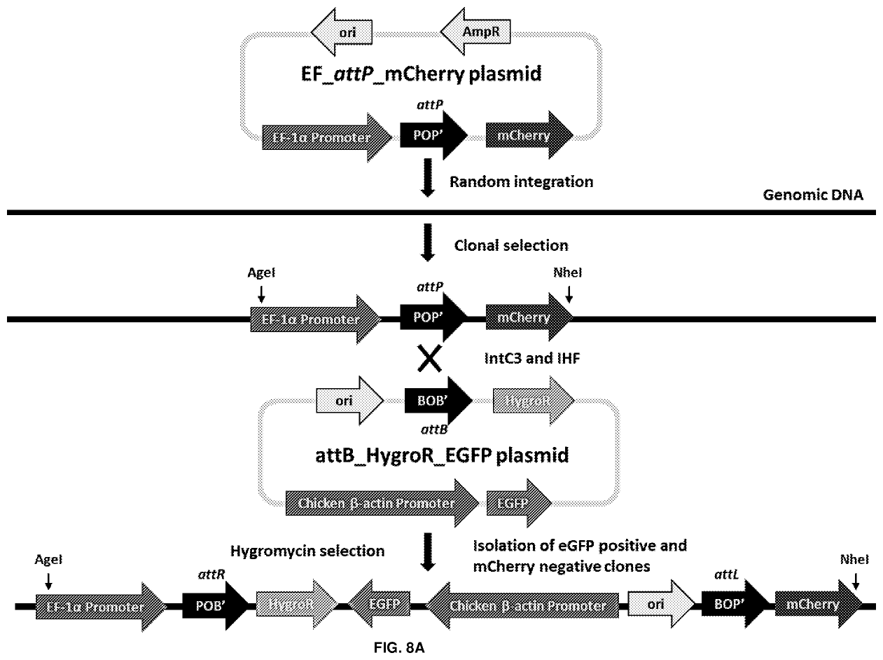

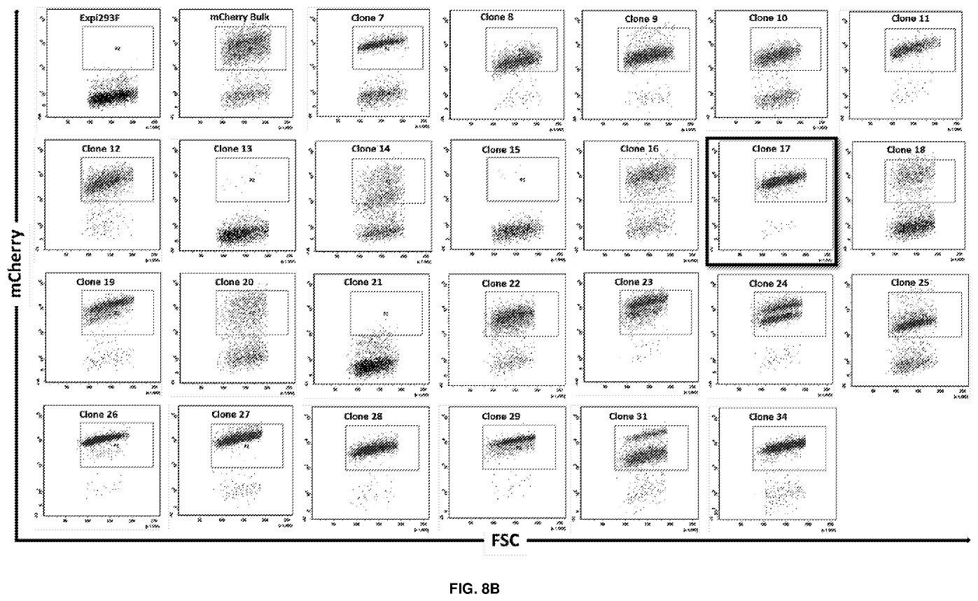

The following strategy was devised to generate clonal Expi293F cell lines containing a single copy landing pad for λ-integrase-mediated insertion of large transgene constructs (FIG. 8A). The eukaryotic elongation factor 1 alpha promoter (pEF-1a) and the coding region for fluorescent protein mCherry flank the recombination target sequence attP (241 bp) in plasmid pEF_attP_mCherry. The attP site lacks translational start codons thus enabling mCherry expression from the upstream pEF-1α promoter.

Expi293F cells in suspension culture were transfected with pEF_attP_mCherry and maintained with regular passages in the absence of selection pressure. mCherry⁺ cells were enriched by several rounds of bulk fluorescent cell sorting. Hence, after random integration, a stable mCherry⁺ expressing bulk cell population was obtained. To generate monoclonal mCherry⁺ cell lines, cells were adapted to adherent growth [50] as adherent cell culture offers advantages in downstream processes like colony picking and expansion of single cells in 96-well plates.

Adherent cells from the mCherry⁺ bulk population were serially diluted to attain single cells which were expanded with the aim to obtain monoclonal cell lines with stable and uniform mCherry expression as analysed by flow cytometry (FIG. 8B). Several clones showed homogenous mCherry⁺ expression, while a few, such as clone #24, showed two populations of cells with distinct mCherry expression levels most likely indicating the presence of two different population of cells each with a different transgene copy number. Based on this analysis, clone #17 was selected due to its high homogeneity and narrow mCherry expression pattern which presumably came from a single copy transgene.

Targeted Integration into attP of Expi293F Cells

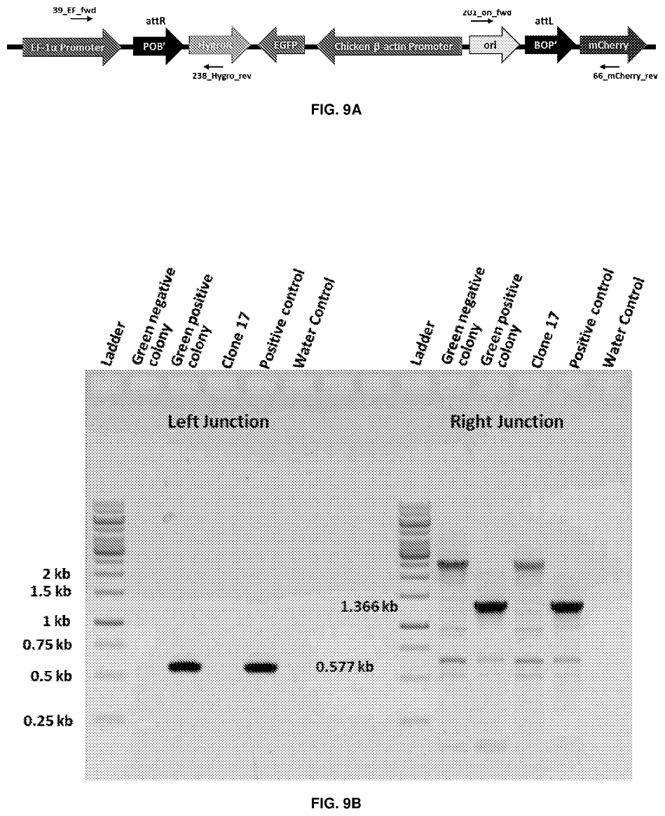

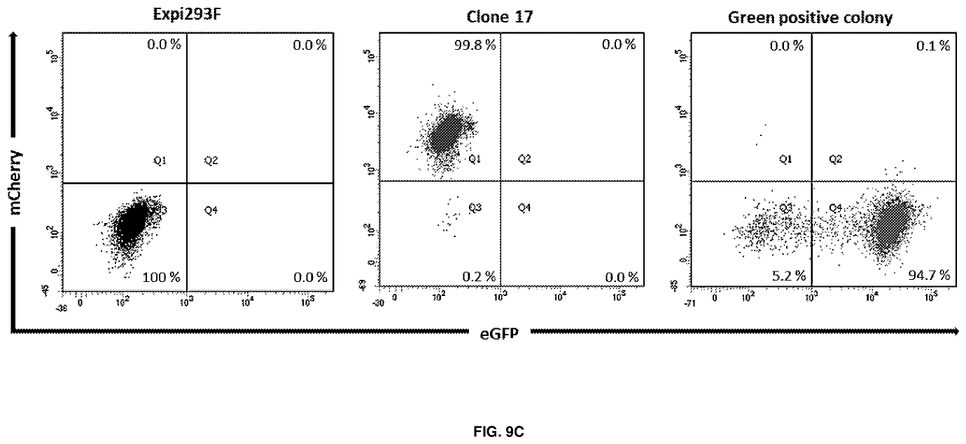

In order to target the attP site in mCherry⁺ cells, a target vector was generated containing an attB site as the corresponding recombination partner sequence (21 bp) for genomic attP and a downstream promoter-less hygromycin resistance gene plus an enhanced green fluorescent protein (eGFP) expression cassette driven by the Chicken β-actin promoter (FIG. 9A; pattB_HygroR-EGFP plasmid). This target vector was co-transfected with expression plasmids for λ-Integrase variant IntC3 and the single chain integration host factor 2 (sclHF2; as an optional IntC3 recombinase co-factor; [21]). Successful integration events catalysed by the recombinase should result in the insertion of the target vector leading to expression of hygromycin^{r} and eGFP⁺, the cessation of mCherry expression, and the creation of two genomic recombination junction sequences attR and attL, as depicted in FIG. 9A.

To demonstrate functionality of the attP site as a transgene landing pad in clone #17, co-transfections were performed followed by hygromycin selection. Surviving colonies were expected to be either double positive for mCherry and eGFP, or single positive for either eGFP or mCherry. The mCherry-expressing cells would most likely entail untargeted cells that carried truncated target vectors resulting in hygromycin resistance. The double positive cells would be either untargeted cells with non-specifically integrated target vectors or successfully targeted cells if there were more than a single landing pad construct present in the genome of parental clone #17 cells.

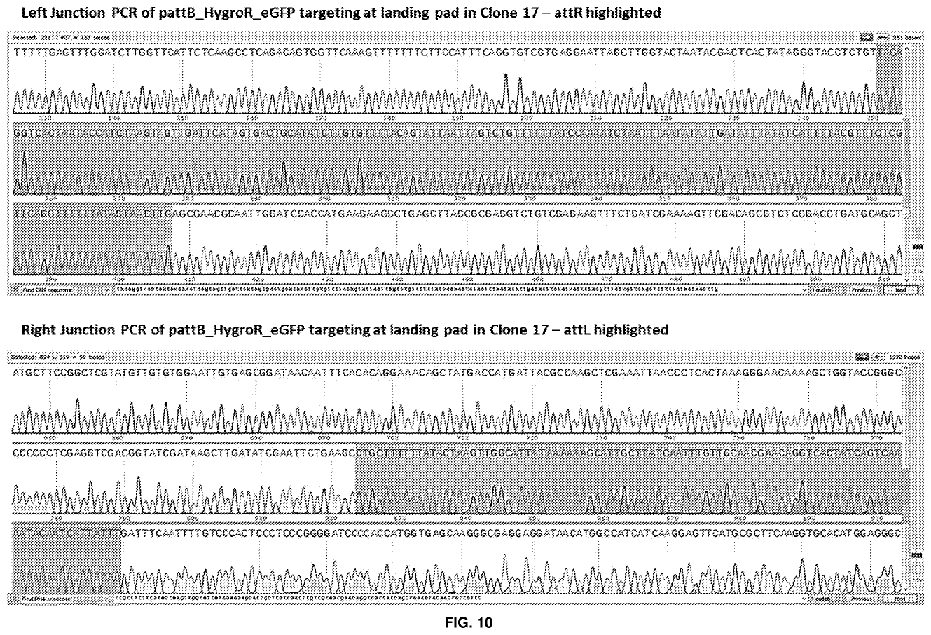

To confirm successful targeting, individual eGFP⁺ and eGFP⁻ colonies were expanded for junction PCR analysis as depicted in FIGS. 9A and 9B. Genomic DNA from these colonies was subjected to PCR analysis using the primers 39_EF_fwd and 238_Hygro_rev for the attR junction, and the primers 201_ori_fwd and 66_mCherry_rev for the attL junction. The presence of a 0.577 kb product (FIG. 9B, Green positive colony; Left Junction) and of a single product of 1.366 kb (FIG. 9B, Green positive colony; Right Junction) confirmed that singly eGFP⁺ cells had been successfully targeted (FIG. 9B). Faithful recombination by IntC3 was confirmed by sequencing of the PCR products (FIG. 10).

Flow cytometry of these eGFP⁺ cells revealed a highly homogenous (>90%) eGFP⁺ cell population and the absence of mCherry⁺ cells (FIG. 9C). It was concluded that upon successful targeting of the attP landing pad in clone #17 cells, eGFP expression completely replaced mCherry expression. This feature can be utilized for the selection of stably targeted cells for future applications.

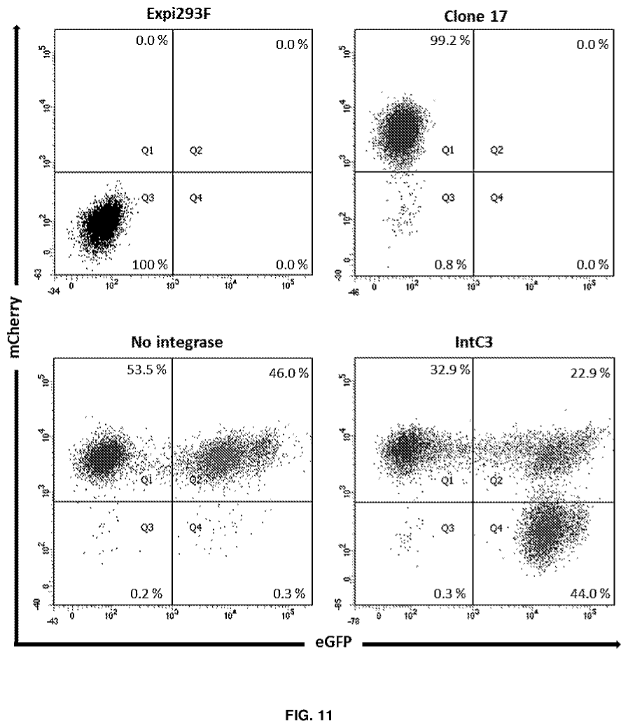

To evaluate the targeting efficiency of attP in #17 cells using IntC3, the same target vector was transfected with and without the IntC3 expression plasmid. After hygromycin selection, cells were analysed as bulk by flow cytometry to determine the fraction of eGFP⁺/mCherry⁻ cells. The results showed that only a negligible number of cells were found to be eGFP⁺ after transfection with target vector alone (FIG. 11, no integrase; Q4), while >40% eGFP⁺ cells were obtained after co-transfection with the IntC3 plasmid (FIG. 11, IntC3; Q4). These data revealed high targeting efficiency of close to half of the hygromycin resistant cell population.

### Expi293 Clone #17 Cells Harbour a Single Functional Landing Pad Site on Chromosome 2

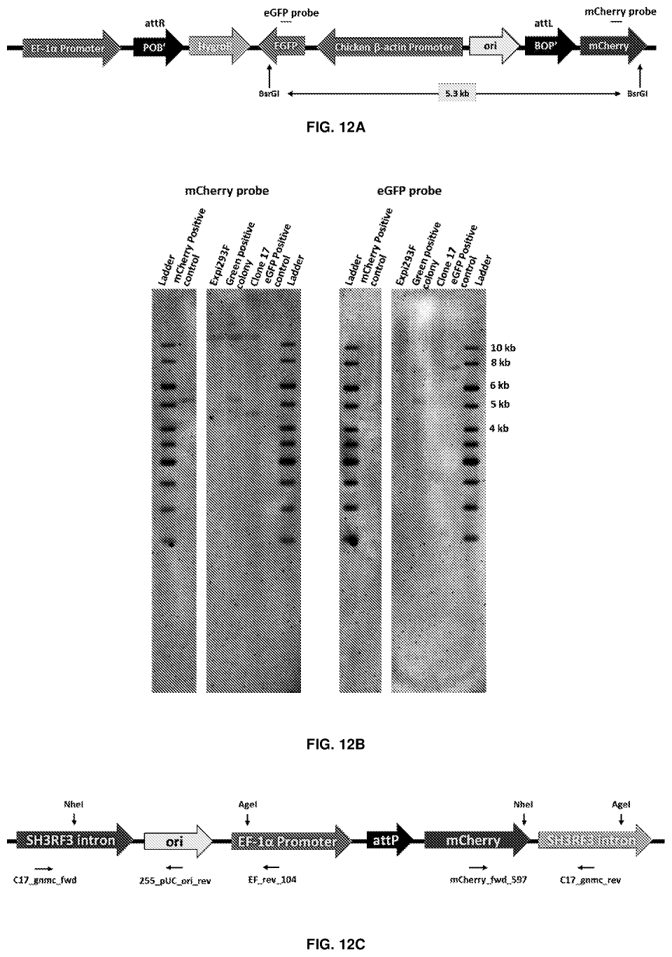

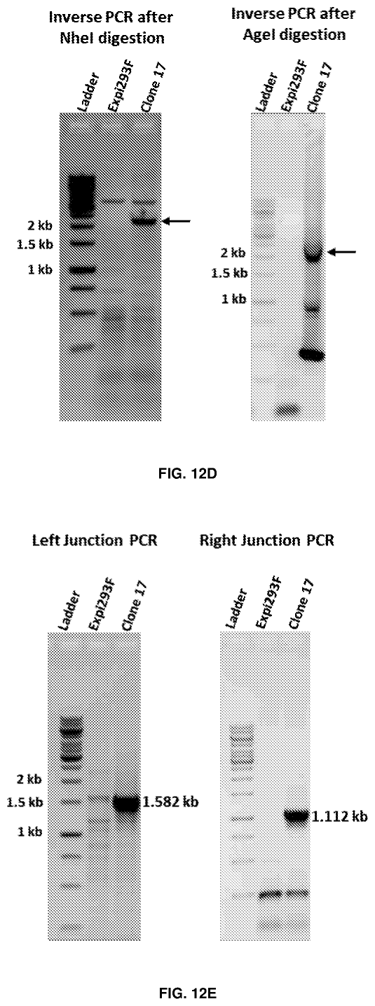

The results indicated that the artificial attP landing pad can be efficiently targeted by IntC3 in clone #17. Southern blot analysis was next performed to determine the copy number of attP sites. Genomic DNA from targeted (eGFP⁺) and untargeted (mCherry⁺) clonal cells was digested with the enzyme BsrGI, which is expected to cut two times in the transgenic DNA after integration of the target vector into the landing pad to generate a 5.3 kb fragment containing the eGFP, Chicken β-actin Promoter, ori, BOP′ and mCherry sequences (FIG. 12A). mCherry and eGFP probes were employed to determine the copy number of landing pads in parental clone #17, as well as the number of transgenes after targeting the landing pad in clone #17 (Green positive colony). As expected, a signal at 5.3 kb genomic fragment size was obtained with both probes in eGFP⁺ cells indicating that a single targeted attP-mCherry cassette is present in the genome (FIG. 12A,B). This conclusion was corroborated by the detection of a fragment of about 4.5 kb (FIG. 12B, mCherry probe, clone #17) using the mCherry probe in the untargeted cells. This fragment resulted from restriction cleavage at the end of the mCherry sequence and at an unknown locus within the genomic DNA. As expected, no fragment was detected in parental untargeted cells using the eGFP probe (FIG. 12B, eGFP probe, clone #17).

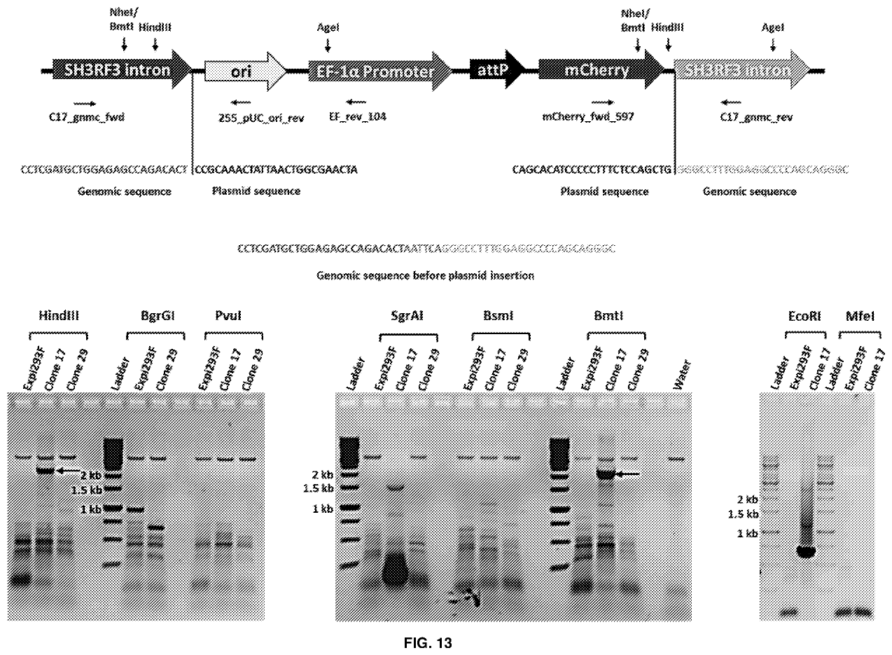

Having demonstrated that parental #17 cells contained one copy of the randomly inserted pEF_attP_mCherry cassette, inverse PCR was employed to identify the exact genomic location of the landing pad cassette. Genomic DNA was digested with either the restriction enzyme Nhel or Agel which have single sites within the landing pad sequence (FIG. 12C), purified and self-ligated, followed by inverse PCR using EF_rev_104 and mCherry_fwd_597 primers. Nhel digestion and self-ligation followed by inverse PCR yielded a >2 kb product (FIG. 12D, Inverse PCR after Nhel digestion, clone #17) which should contain flanking genomic sequences located 5′ of the EF-1α promoter. Agel digestion and self-ligation followed by inverse PCR yielded a ˜2 kb long product (FIG. 12D, Inverse PCR after Agel digestion, clone #17) which should contain flanking genomic sequences located 3′ of the integrated mCherry coding sequence. Sequencing of these PCR products revealed the same genomic locus as integration site of the landing pad cassette. Inverse PCR analyses after genomic DNA digestion with other restriction enzymes further corroborated these results (FIG. 13). Subsequent nucleotide sequence alignments revealed that the genomic locus of the landing pad cassette was identified in the third intron of the SH3 Domain Containing Ring Finger 3 gene (SH3RH3) on chromosome 2 (FIG. 12C, SH3RF3 intron), see also Table 2 below. The sequence alignment also revealed that the random pEF_attP_mCherry cassette integration resulted from a DNA double strand break in that intron with loss of only six nucleotides as depicted in FIG. 13.

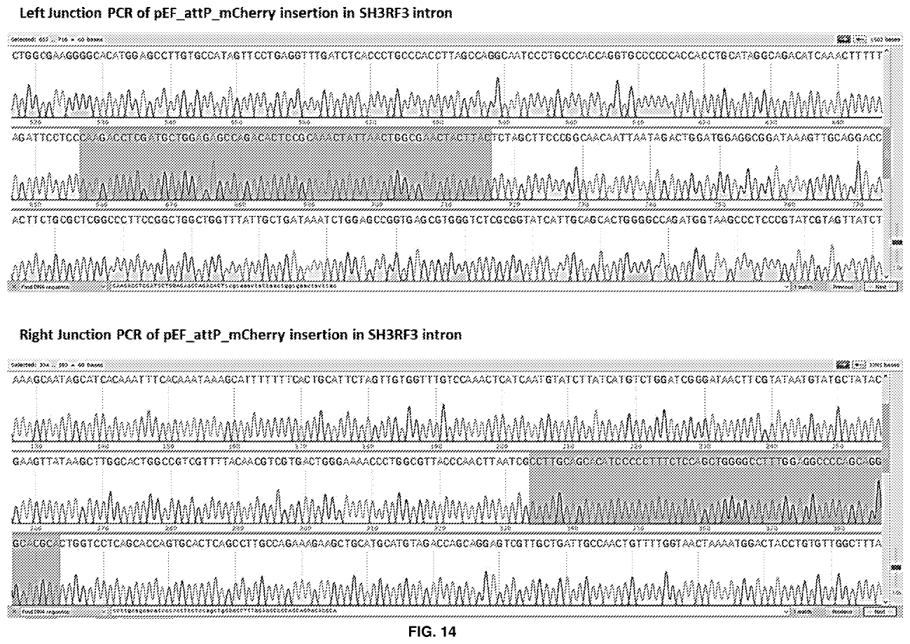

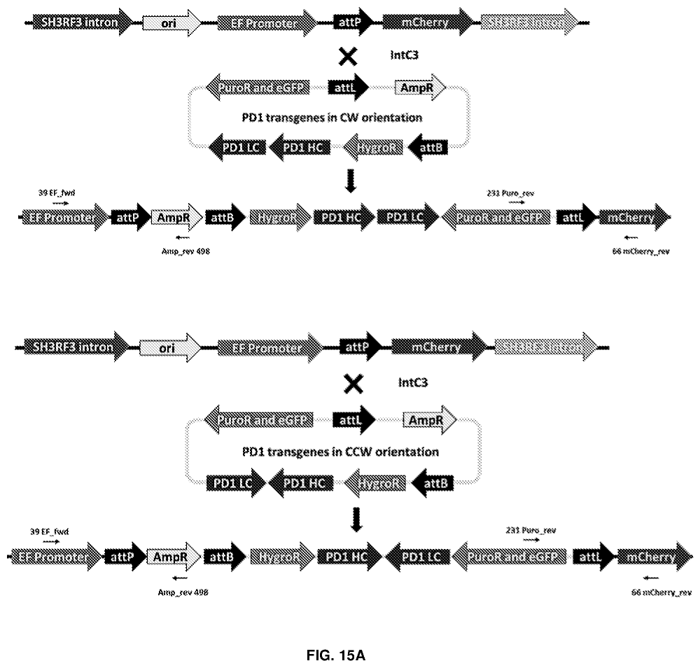

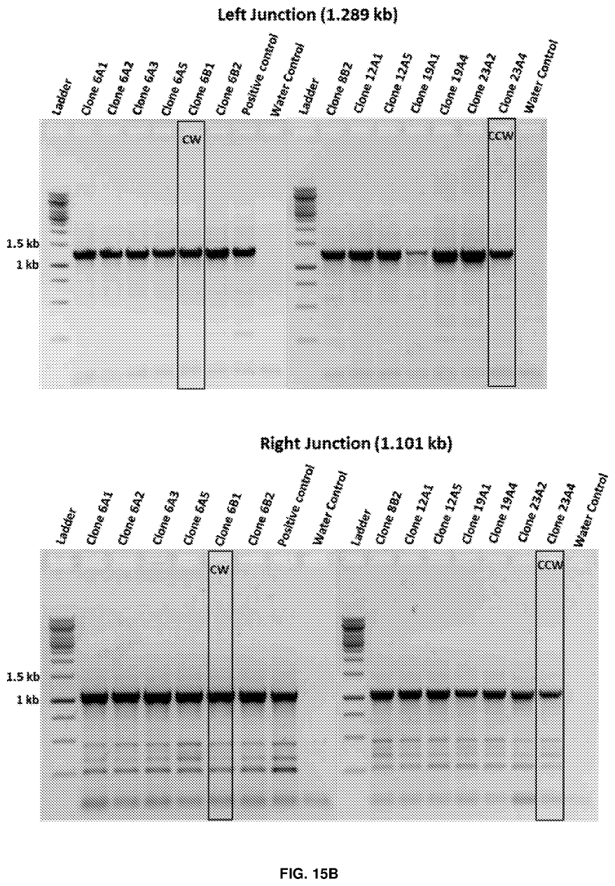

To directly confirm the sequence accuracy of the targeted genomic locus for landing pad insertion, direct genomic junction PCRs were performed on genomic DNA using primers C17_gnmc_fwd located in the SH3RH3 intron along with primer 255_pUC_ori_rev to obtain the predicted size for the left junction PCR product of 1.582 kb (FIG. 12E; Left Junction PCR, clone #17), and by using primers C17_gnmc_rev located in the SH3RH3 intron along with primer mCherry_fwd_597 to obtain the predicted size for the right junction PCR product of 1.112 kb (FIG. 12E; Right Junction PCR, clone #17). Sequencing results confirmed the site of insertion and break points in the plasmid (FIG. 14). It was concluded that this genomic locus can serve as a human safe harbor site for long-term transgenesis expression.

TABLE 2

| SEQ ID NO: | Description | Nucleotide sequence |
|---|---|---|
| 32 | Genomic safe harbor | GTTGTGTCTATGTCTGAACAGAGTGGTCCAGGAGGCAA |
| | targeting locus on | ATTCTTAGACCTATCAGGGAGAATAAGCAGTGGTCTTG |
| | human chromosome | CCTTCACCAGTAACCCACTGTTTCCCTTACCTTTCAATT |
| | #2 | GGATTAACGACATAAAGAAATAAATGAGTGTAAAGAAA |
| | Source: Homo sapien | TGAGACAAGACTACATAAGAGGGAGACTCCAGTCGCAT |
| | | TCATGGTTGGGGTCTGTACATTTTCTTGCCGGGATCTG |
| | | ACTGCCAAGGCGTGATCCACACATACATGGGTGTTCAT |
| | | TGTAGTTTGGGTTTGTTTGTCTCCAGCAAAAATAGGTA |
| | | GGTGCCTAGGTACCGGCTCGCCCTGATTTCTCTCTTCT |
| | | TAAGTCTTTTTGAGGTCTTCGTGAAATTAACATCTTATA |
| | | ATAGAATATCTTACATTCCATTGCTGCTGGGAGTCTTGT |
| | | TGCTGTGATTGTTTTGTCTCCCTAAGCCCCTTTGAGGC |
| | | CAGACCTGTAACTCACAGTTGCTTCCCTTCTGGGTCCT |
| | | GGCACAGGGTGGGGCCCAGGTGCTAGCTACCCTGTG |
| | | CACCTGTGCAATGTTATTGGGTTGTGGTTCAGTGTTTTA |
| | | CAGAGCTCATCAAGCATGGGGAATGCCCCACTGACCA |
| | | TCTCTAAATGGGTCTAAGCTGGGAAGCTTGCAGCAGTT |
| | | CTTGTATATCTGTGGCAAGACAGCTTAGTAAGTTTTTGT |
| | | GAAGCCCCTTGTCCTCGCTGCCAGCCTCCCTGGGAGC |
| | | TTCTGTGGGGACAGTGAGTAGCCCTGACCGCAGCCTG |
| | | AGTGGCTCAGTCATCACTGGGGCCACAGCCCACCTTA |
| | | AATGTTGTCAGGGCCTGGCGAAGGGGCACATGGAGCC |
| | | TTGTGCCATAGTTCCTGAGGTTTGATCTCACCCTGCCC |
| | | ACCTTAGCCAGGCAATCCCTGCCCACCAGGTGCCCCC |
| | | CACCACCTGCATAGGCAGACATCAAACTTTTTAGATTC |
| | | CTCCCAAGACCTCGATGCTGGAGAGCCAGACACTAATT |
| | | CAGGGCCTTTGGAGGCCCCAGCAGGGCACGCACTGG |
| | | TCCTCAGCACCAGTGCACTCAGCCTTGCCAGAAAGAA |
| | | GCTGCATGCATGTAGACCAGCAGGAGTCGTTGCTGATT |
| | | GCCGACTGTTTTGGTAACTAAAATGGACTACCTGTGTT |
| | | GGCTTTAAGCAGTAGTTGTGAAATTGTGTGTCAGGAAG |
| | | AGGGCTTCCTCCACGGGTCTACCAAGTGGTCTACCACT |
| | | TGCTGAGTGCAGAGCCTTGTCCTGGTCTGGATGCGAC |
| | | GATGGAAGGCTTAAGTGAGTCATCTCCTGAAGAGACTA |
| | | CTCAGGACTGGAAGTGTGTCAGCCTAGGGGCAGCTGA |
| | | ACACCCAGGTGGCCCTGGAGCAGGCTGCTGGAGCGT |
| | | CTGGCCTGCAGCCGGTGGAAGTAACTGAGCTCCAATC |
| | | TTGTGTCCAGACTGCTTCTCCCCTAGGCTCCAGCTGTT |
| | | TTTCTGCTTCTCCTGTCACTCATATGGACAGTGCACTTT |
| | | CCACTGAGCAGGTCAATGGCGGGGTTACTTTTACACCT |
| | | AACTTTAGCTCCTCTCTGGGCCCTGCTGGTCTCAGCAC |
| | | GCACACCCACCCAATCCTACGGAGACAGAGTTCTCACA |
| | | CCTACCCTCCGCAGGGCATCTTGGGCCATCCACGTCC |
| | | ACCTGCCCTCCCTTCAGCGTGGCCATCACGGTGCCCT |
| | | GACTCAGCTTCCAGTCCCCCGGGCTTCCATGTTCTTGT |
| | | CTTCCCTTTCACTTCTACCTTTCCTGCTTGAGTAAGTTA |
| | | GGCTGTGGCTCGCAAACTTCAGTGAGCATTTGAGTGTT |
| | | GTCAGAAGGCCCCCAGGATGGCTAAATAGTAGAAAGG |
| | | ACAGCTTTACTGGTGACATCAGTTTGCAAGCCAGGAAT |
| | | AGATAGTCTCTGACATGGAACGAAGGTGCTCTCGCTTC |
| | | GCAGAAGGGGAAGGGCAGTTGGATTTTATGGCTCGCA |
| | | GGGTCTGTGTTACATAGTAGAATCAGACATGTTTAGCA |
| | | GGTTTGGGGGAAAGCTG |

### Targeted Expression of Monoclonal Anti PD-1 Antibodies in Expi293F Cells

To exemplify utility of the Expi293F transgenic cell platform, complex multi-transgene target vectors were generated harbouring anti-PD-1 human IgG heavy and light chain monoclonal antibody genes [22] expressed from different promoters. Heavy (PD-1 HC) and light (PD-1 LC) chains were placed in two orientations with respect to each other, i.e. head-to-tail (CW) or head-to-head (CCW) (FIG. 15A), to evaluate a possible impact on antibody expression. In addition, these targeting plasmids also contain a puromycin resistance gene for stringent selection, an eGFP gene as a marker and an attL recombination site. The promoter-lacking hygro^{r} gene is located downstream of a second recombination site, attB. These plasmids were designed to simultaneously test which recombination site pairing (i.e. genomic attP×vector attB; vector attL×vector attB; genomic attP×vector attB) would ultimately yield targeted genome insertion in parental clone #17 cells. IntC3-mediated intramolecular recombination between attL and attB sites that occur before intermolecular genomic insertion would result in seamless vectors as previously described [23].

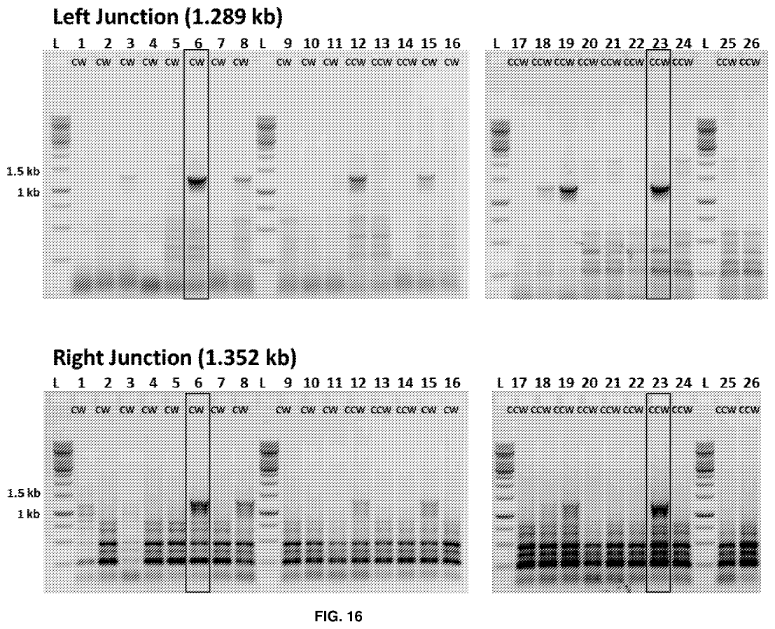

Adherent #17 cells were co-transfected with PD-1 target vectors (antibody genes either in CW or CCW orientation) and IntC3 plus scIHF2 expressing plasmids. After antibiotic selection, 26 colonies that initially appeared eGFP⁺ and mCherry− were isolated, expanded and screened for attP-targeted genomic integration using specific PCR primer combinations that considered the different possibilities of att site pair utilization as mentioned above. Targeted attP site transgene insertion on chromosome 2 in eight colonies was confirmed, with four colonies carrying the PD-1 genes in CW orientation and four colonies in CCW orientation (FIG. 16). Surprisingly, it was found that all eight carried the transgene cassette as a result of IntC3-mediated attP (genome)×attL (vector) recombination events (FIG. 15A). This result thus revealed a very strong preference for this pair of att sites for targeted genome integration in attP since parallel PCR screening of the 26 colonies for attP (genome)×attB (vector) recombination events produced no positive results.

### Impact of IgG Chain Gene Arrangement on Antibody Expression

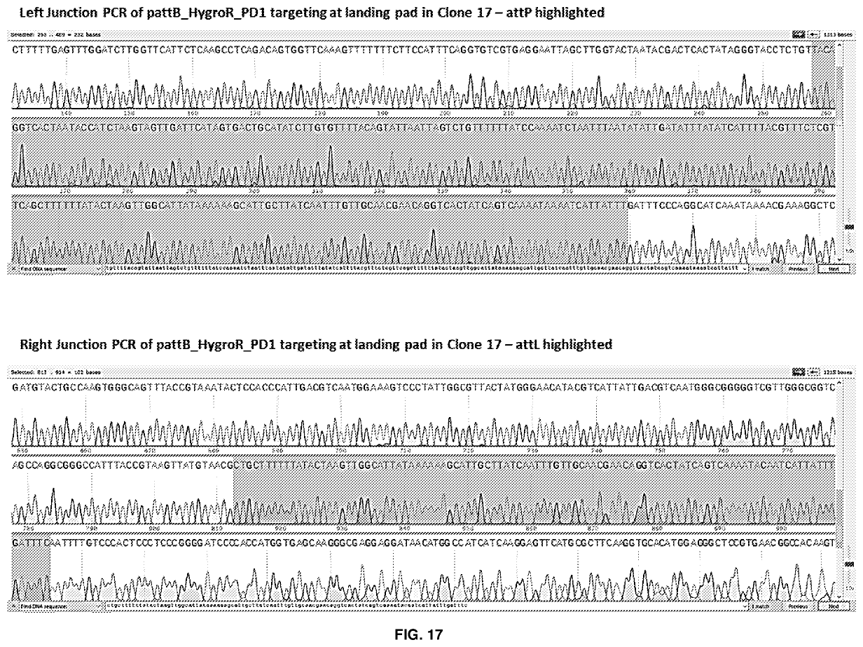

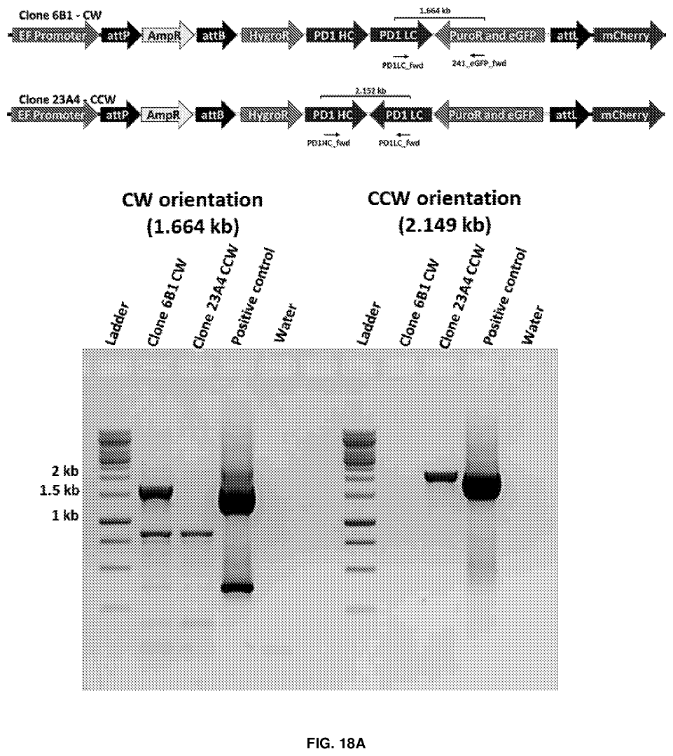

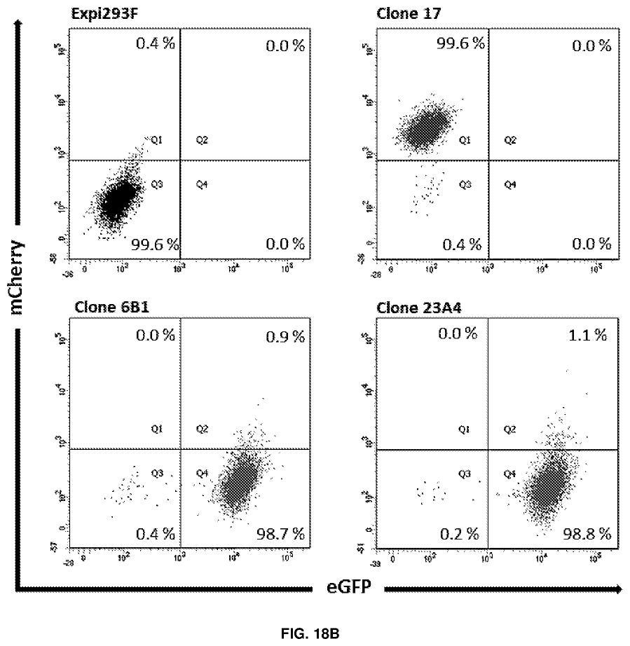

Antibiotic selection was removed from targeted colonies, and pure cell sub-clones from both target vectors were obtained by two-fold serial cell dilution from clones 6 (CW) and 23 (CCW) (FIG. 16). Faithful transgene insertion resulting from attP (genome)×attL (vector) site-specific recombination was confirmed in all 13 sub-clones analysed by PCR/sequencing of both junctions (FIG. 15B and FIG. 17). One individual sub-clone obtained with each target vector, i.e. #6B1 (CW) and #23A4 (CCW), was selected for detailed PD-1 antibody expression analysis.

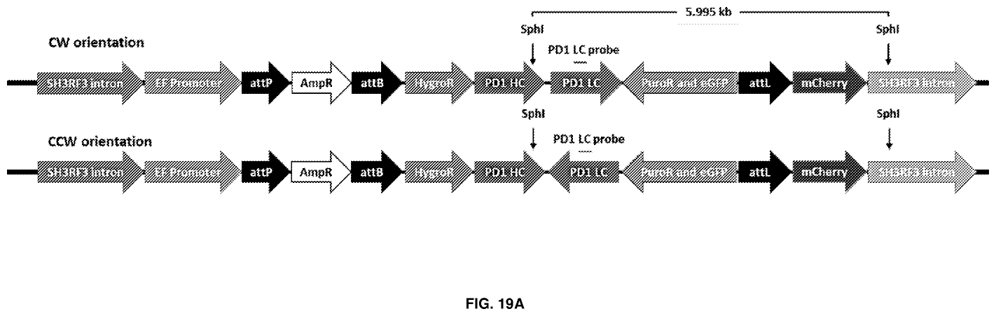

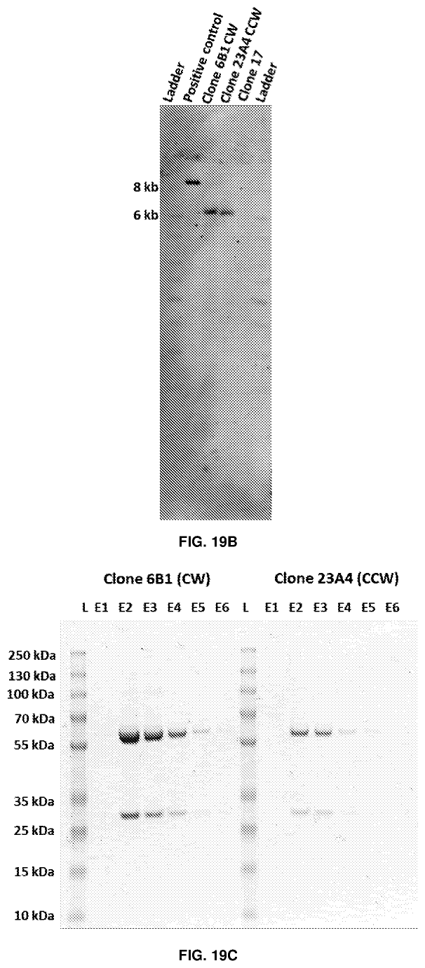

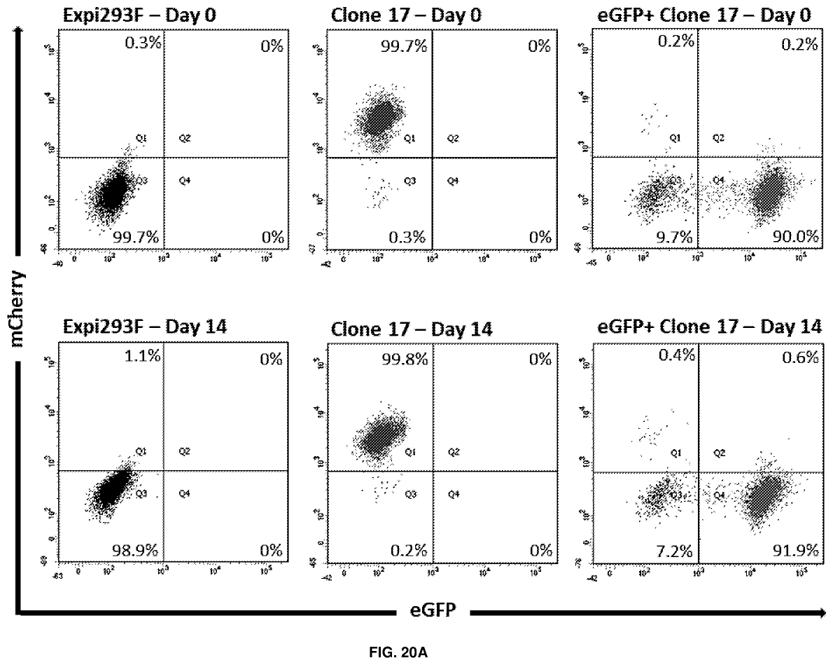

IntC3 has previously been shown to site-specifically insert large (>8 kb) plasmids into the human genome [20]. Here, it has been demonstrated that a >10 kb plasmid containing 5 transgenes could be successfully delivered. To confirm the accuracy of the inserted PD-1 transgene constructs, PCR and sequencing analysis was used on the two selected sub-clones #6B1 and #23A4 and revealed that both clones had the correct internal sequence indicative of their transgene orientations (CW or CCW) without cross-contamination (FIG. 18A). Both cell clones were further subjected to Southern blot analysis to verify single copy plasmid integration at the genomic attP target site on chromosome 2 using a IgG light chain gene probe. Genomic DNA was digested with Sphl which yields a single diagnostic fragment of 5.995 kb for both PD-1 gene orientations (FIG. 19A). This fragment was detected in both analysed subclones (FIG. 19B, clone 6B1 and clone 23A4) but not in the parental cell line containing the untargeted landing pad (FIG. 19B, clone 17). The appearance of a single fragment thus confirmed single copy integration since the downstream Sphl site lies within the predicted genomic (SH3RF3 intron) sequence flanking the landing pad.

Levels of antibody secretion were next compared for the two selected subclones which differed in heavy and light chain gene arrangements but were otherwise isogenic. Secreted IgG was affinity purified from the cell culture medium via stepwise elution from protein G agarose resins. The purity of eluted fractions were analysed by SDS-PAGE (FIG. 19C; E lanes), and the total combined IgG yield determined by spectrophotometry. Isolated IgG from both clones was found to be homogeneous without visible contaminations. It was found that from a small suspension culture volume (100 ml), IgG yield from clone #6B1 was about 4-fold higher than from clone #23A4 (41.75±0.25 μg versus 10.35±1.25 μg) suggesting that significantly higher antibody production can be achieved when the two transcription units are oriented as direct (head-to-tail) repeats as opposed to the inverted (head-to-head) orientation at the same genomic locus. Both cell sub-clones were also analysed by flow cytometry and were found to be more than 98% single eGFP⁺ (FIG. 18B).

### Long-Term Homogeneous and Selection-Free Transgene Expression

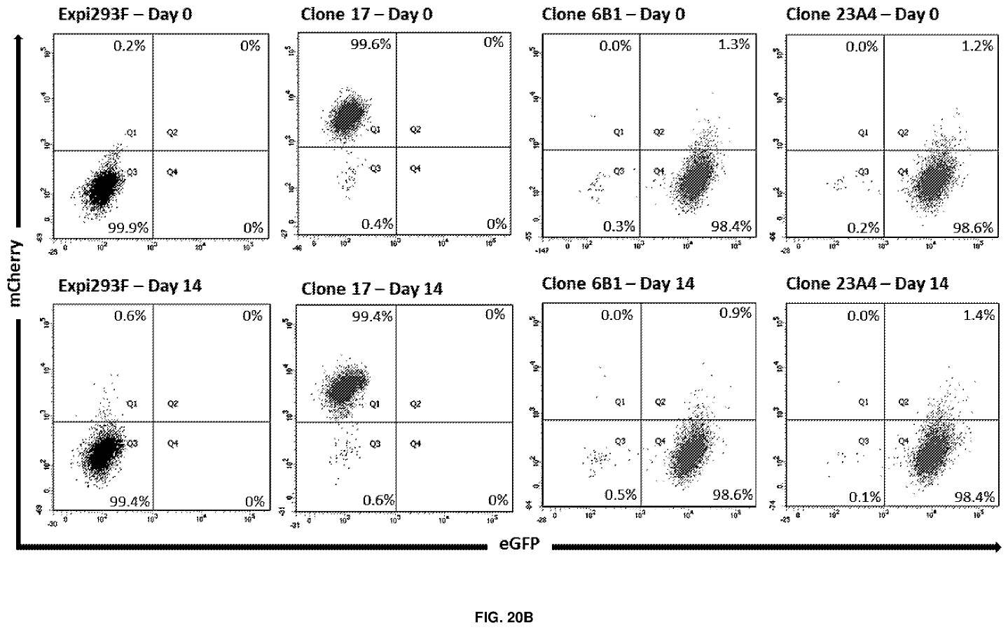

For several downstream applications, it is critical that cells harbouring transgenes remain stable over long periods in the absence of any selection pressures. To determine genetic and epigenetic stability, cells from a pattB HygroR_eGFP targeted colony were examined by flow cytometry before and after 14 days of continuous culture and were found to maintain homogeneity at >90% (FIG. 20A). Similarly, FACS analysis showed sustained and homogenous (>98% eGFP⁺) in PD-1 antibody expressing sub-clones #6B1 and #23A4 after 14 days of culture without selection (FIG. 20B), indicating that integration at this landing pad locus on chromosome 2 is genetically stable as well as transcriptionally active over a long periods of time. In fact, since the generation of these PD-1 expressing subclones was already performed in the absence of any selection pressure over more than six weeks, the transgene expression from this genomic locus remains unchanged over several months.

IgG Secretion from Encapsulated Expi293F Platform Cells

The Expi293F #17 cell platform can be used to engineer cells for perennial expression and secretion of biologics. Although the transgene's genomic locus is well defined and genetically and functionally stable, expression levels from a single transgene will generally be lower than transient expression from multi copy episomal plasmids. However, finely tuned and steady expression could become important for other applications, such as cell therapy using mini-bioreactors comprising encapsulated cells engineered for therapeutic secretion. These mini-bioreactors can secrete and accumulate biotherapeutics at high concentrations at the site of transplantation in the body. Encapsulation of cells prevents their escape from the site of implantation and it also protects them from the patient's immune responses. The capsules also act as a safety device as they protect the patient from the foreign cells implanted.

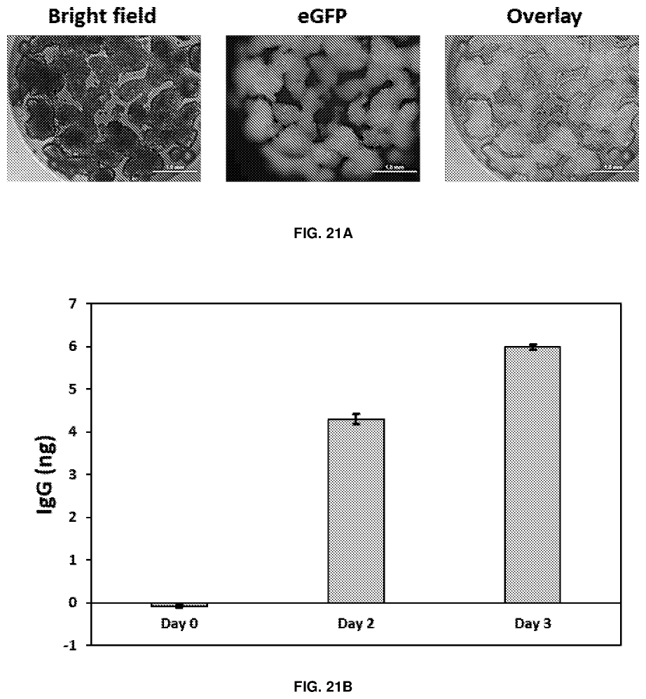

Using a cellulose sulfate-based encapsulation strategy (Cell-in-a-Box®), we encapsulated anti-PD1 antibody producing #6B1 cells and examined individual capsules for eGFP expression. As shown in FIG. 21A with one isolated capsule as an example, eGFP+ cells which had been encapsulated at low cell density of about 5 k cells/capsule continued to proliferate and eventually occupied previously empty spaces within capsules. Secretion of human IgG from the encapsulated cells into the media was quantified by ELISA and revealed a steady 50% increase of IgG secretion per day (FIG. 21B).

### DISCUSSION

The rapid emergence of novel biologics warrants more time and cost-effective cell-based tools for production. In this context, GMP-grade Expi293F cells of human origin are an increasingly attractive alternative to the currently used CHO cell lines. The development of Expi293F cell lines combined with safe genome editing tools to produce master clones provides additional advantages and has been the aim of the present invention.

Targeted genome manipulation by site-specific recombinases such as IntC3 employed here can enable precise, locus-specific knock-in of transgene expression constructs into stable and well-characterized sites in the host cell genome. As disclosed herein, a strategy has been devised to first select cell clones based on homogenous and stable fluorescent reporter expression in the absences of any selection pressure. This provided a simple screening method to subsequently derive master cell lines carrying a functional single copy artificial transgene landing pad, attP, by switching off fluorescent marker expression and establishing conditional antibiotic resistance after recombinase-mediated transgene insertion.

As a proof of concept, a master Expi293F cell line #17 which carries attP in an intron of the SH3RF3 gene on chromosome 2 has been fully characterized. The SH3 domain containing ring finger 3 protein, SH3RF3, has E3 ubiquitin-protein ligase activity and is involved in JNK signalling and protein autoubiquitination [24,25]. The corresponding mRNA is found to be enriched in a few cell types such as in the kidney [26]. There appears to be no compelling evidence that aberrant expression of this protein is causally linked to human pathologies, which renders the unique attP site in these cells as a potential safe harbour for transgene insertion. Furthermore, the characterization of targeted cells clearly revealed long-term genetic transgene stability and expression from the targeted genomic locus which only lacks six endogenous nucleotides in the SH3RF3 gene. The availability of GMP-grade Expi293F cells in combination with programmable endonuclease-induced homologous recombination should enable the future generation of a GMP-based #17 master cell line homologue for clinical applications.

As an example of the utility of the exemplified #17 Expi293F cell line, two distinct large (>10 kb) transgene vectors were inserted for monoclonal anti PD-1 antibody expression into the genomic landing pad attP. It was confirmed that resulting cell subclones carried only one intact copy of the respective transgene construct which differed solely in the orientation of heavy and light chain transcription units with respect to each other. The quantitative antibody expression/secretion analysis revealed that a 4-fold higher antibody production is achieved when the two transcription units are oriented as direct (head-to-tail) repeats as opposed to the inverted (head-to-head) orientation at the same genomic locus. A possible explanation for such a pronounced difference could be transcription-induced DNA supercoiling which would lead to high levels of positive DNA supercoil accumulating in front of the two translocating RNA polymerases moving towards each other in the head-to-head gene orientation. This, in turn, may lead to inhibition of mRNA synthesis.

Furthermore, the targeting of the single copy attP site with the foregoing antibody expression constructs revealed that the preferred recombination partner sequence for the genomic attP on the incoming target vector is attL. This was demonstrated by placing both attB, which is the natural recombination partner for attP in the wild-type phage lambda recombination system, and attL on the target vector thus creating competition for recombination with attP on chromosome 2. While it was clearly demonstrated that attB can be used efficiently by IntC3 to recombine with attP in #17 cells when there is no attL site present (FIG. 14), it appears that attL under competing conditions is the preferred attP partner. Likewise, attL appears to preferentially recombine in trans with genomic attP instead of attB, even when attB is present in cis on the same DNA molecule.

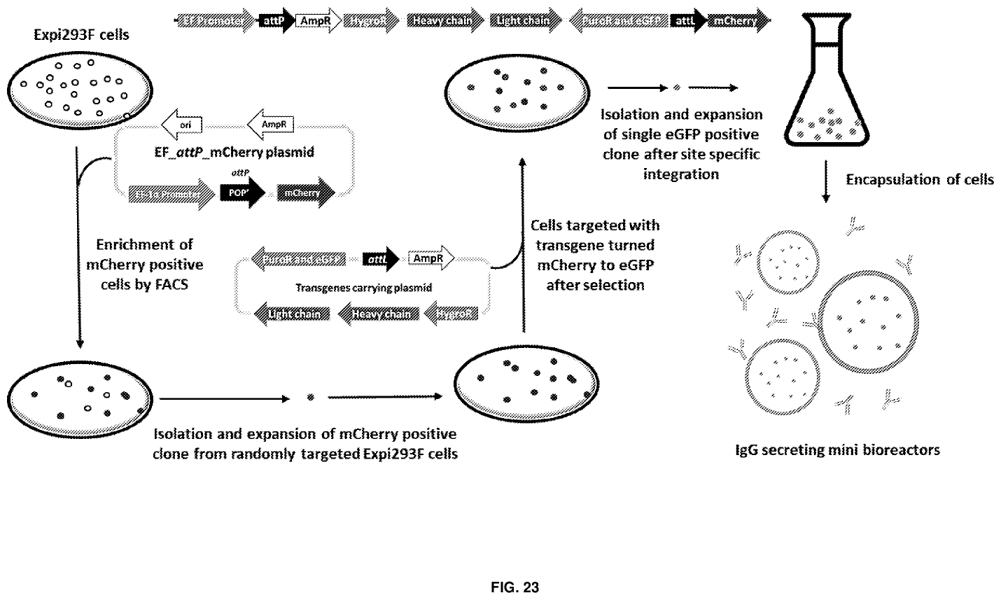

For future therapeutic purposes, anti PD-1 antibody expressing cells were encapsulated to create cellulose-based mini-bioreactors and confirmed continued, steady IgG secretion from the capsules. FIG. 23 illustrates a graphical representation of the process steps in producing said cellulose-based mini-bioreactors.

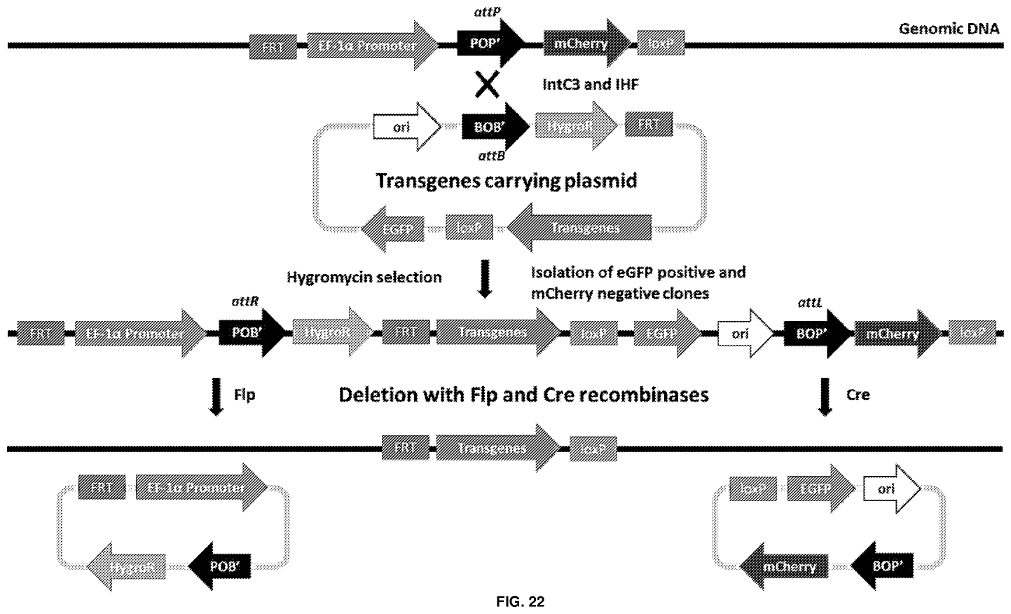

Future applications could utilize allogeneic transplantation of such bioreactors into a patient's body to deliver, for example, high antibody concentrations near sites of malignant cell growth. Considering the high costs of immunotherapies currently in clinical practice, mini-bioreactors could provide a safe and cost-effective alternative and have recently been shown to work efficiently in animal models [27]. In this context, the core platform disclosed herein offers future inclusion of additional site-specific recombinases as part of a modular system. For example, transgenic cell lines lacking remaining marker sequences for bioreactor production could easily be obtained by co-placing directly repeated pairs of each recombinase cognate sites loxP and FRT in the transgenic locus on chromosome 2. The Cre and Flp recombinases expressed from transfected mRNA can then excise undesirable DNA segments as modelled in FIG. 22. In addition, a single cognate sequence for the serine recombinase Bxb1, which works efficiently in human cells, can be included in the design. After IntC3-mediated recombination into the genomic attP, Bxb1-mediated intermolecular recombination can deliver an additional transgene construct into the same genomic locus to achieve, for example, tightly regulated large scale expression of (multi-)transgenes that exhibit severe mammalian cell toxicity.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention.

The methods and specific cells described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

[1] Tripathi N K, Shrivastava A. Recent Developments in Bioprocessing of Recombinant Proteins: Expression Hosts and Process Development. Front Bioeng Biotechnol 2019; 7:420.
[2] Kuriakose A, Chirmule N, Nair P. Immunogenicity of Biotherapeutics: Causes and Association with Posttranslational Modifications. J Immunol Res 2016; 2016.
[3 ] Yang Y, Mariati, Chusainow J, Yap M G S. DNA methylation contributes to loss in productivity of monoclonal antibody-producing CHO cell lines. J Biotechnol 2010; 147:180-5.
[4 ] Spencer S, Gugliotta A, Koenitzer J, Hauser H, Wirth D. Stability of single copy transgene expression in CHOK1 cells is affected by histone modifications but not by DNA methylation. J Biotechnol 2015; 195:15-29.
[5 ] Fang X T, Sehlin D, Lannfelt L, Syvanen S, Hultqvist G. Efficient and inexpensive transient expression of multispecific multivalent antibodies in Expi293 cells. Biol Proced Online 2017; 19:11.
[6 ] Ayuso E. Manufacturing of recombinant adeno-associated viral vectors: new technologies are welcome. Mol Ther Methods Clin Dev 2016; 3:15049.
[7 ] Salmons B, Hauser 0, GOnzburg W, Tabotta W. GMP Production of an Encapsulated Cell Therapy Product: Issues and Considerations. BioProcessing Journal 2007; 6:37-44.
[8 ] Kim T K, Eberwine J H. Mammalian cell transfection: the present and the future. Anal Bioanal Chem 2010; 397:3173-8.
[9 ] Chong Z X, Yeap S K, Ho W Y. Transfection types, methods and strategies: a technical review. PeerJ 2021; 9.
[10 ] Kim M, O'Callaghan P M, Droms K A, James D C. A mechanistic understanding of production instability in CHO cell lines expressing recombinant monoclonal antibodies. Biotechnol Bioeng 2011; 108:2434-46.
[11 ] Malphettes L, Freyvert Y, Chang J, Liu P Q, Chan E, Miller J C, et al. Highly efficient deletion of FUT8 in CHO cell lines using zinc-finger nucleases yields cells that produce completely nonfucosylated antibodies. Biotechnol Bioeng 2010; 106:774-83.
[12 ] Lee J S, Grav L M, Lewis N E, Kildegaard H F. CRISPR/Cas9-mediated genome engineering of CHO cell factories: Application and perspectives. Biotechnol J 2015; 10:979-94.
[13 ] Sakuma T, Takenaga M, Kawabe Y, Nakamura T, Kamihira M, Yamamoto T. Homologous Recombination-Independent Large Gene Cassette Knock-in in CHO Cells Using TALEN and MMEJ-Directed Donor Plasmids. Int J Mol Sci 2015; 16:23849-66.
[14 ] Turan S, Galla M, Ernst E, Qiao J, Voelkel C, Schiedlmeier B, et al. Recombinase-mediated cassette exchange (RMCE): Traditional concepts and current challenges. J Mol Biol 2011; 407:193-221.
[15 ] Zhou R, Droge P. High-Precision Genome Surgery in Human Stem Cells. Curr Genomics 2006; 7:427-33.
[16 ] Hamaker N K, Lee K H. Site-specific Integration Ushers in a New Era of Precise CHO Cell Line Engineering. Curr Opin Chem Eng 2018; 22:152-60.
[17 ] Siau J W, Chee S, Makhija H, Wai C M M, Chandra S H V, Peter S, et al. Directed evolution of λ integrase activity and specificity by genetic derepression. Protein Eng Des Sel 2015; 28:211-20.
[18 ] Makhija H, Roy S, Hoon S, Ghadessy F J, Wong D, Jaiswal R, et al. A novel λ integrase-mediated seamless vector transgenesis platform for therapeutic protein expression. Nucleic Acids Res 2018; 46.
[19 ] Crescioli S, Chiaruttini G, Mele S, Ilieva K M, Pellizzari G, Spencer D I R, et al. Engineering and stable production of recombinant IgE for cancer immunotherapy and AllergoOncology. J Allergy Clin Immunol 2018; 141:1519-1523.e9.
[20 ] Vijaya Chandra S H, Makhija H, Peter S, Myint Wai C M, Li J, Zhu J, et al. Conservative site-specific and single-copy transgenesis in human LINE-1 elements. Nucleic Acids Res 2016; 44.
[21 ] Corona T, Bao Q, Christ N, Schwartz T, Li J, Dröge P. Activation of site-specific DNA integration in human cells by a single chain integration host factor. Nucleic Acids Res 2003; 31:5140-8.
[22 ] Wang, Cheng-I; Oh, Hsueh Ling Janice; Yeo, Siok Ping; Low, Jianrong Lionel; Tan H C. Anti-pd-1 antibodies, 2015.
[23 ] Makhija H, Roy S, Hoon S, Ghadessy F J, Wong D, Jaiswal R, et al. A novel λ integrase-mediated seamless vector transgenesis platform for therapeutic protein expression. Nucleic Acids Res 2018; 46.
[24 ] Fu S, Ma C, Tang X, Ma X, Jing G, Zhao N, et al. MiR-192-5p inhibits proliferation, migration, and invasion in papillary thyroid carcinoma cells by regulation of SH3RF3. Biosci Rep 2021; 41.
[25 ] Zhang P, Liu Y, Lian C, Cao X, Wang Y, Li X, et al. SH3RF3 promotes breast cancer stem-like properties via JNK activation and PTX3 upregulation. Nat Commun 2020; 11.
[26 ] Uhlén M, Fagerberg L, Hallström BM, Lindskog C, Oksvold P, Mardinoglu A, et al. Tissue-based map of the human proteome. Science (1979) 2015; 347.
[27 ] Bose S, Volpatti L R, Thiono D, Yesilyurt V, McGladrigan C, Tang Y, et al. A retrievable implant for the long-term encapsulation and survival of therapeutic xenogeneic cells. Nat Biomed Eng 2020; 4:814-26.

1. A method for production of a eukaryotic host cell or cell line comprising a landing pad for λ-integrase-mediated recombination, the method comprising:
(i) transfecting a bacterial plasmid into a eukaryotic host cell at suitable conditions to induce said transfection, wherein the bacterial plasmid comprises a landing pad comprising:
a. an RNA polymerase promoter;
b. a modified lambda integrase recombination sequence attP or derivative thereof, downstream of the RNA polymerase promoter, wherein the modified phage lambda integrase recombination sequence has been modified to remove ATG start codons; and
c. a first fluorescent marker gene downstream of the modified lambda integrase recombination sequence and operably linked to the RNA polymerase promoter;
(ii) culturing the transfected eukaryotic host cell under conditions that allow expression of the first fluorescent marker gene;
(iii) identifying a eukaryotic host cell into which the bacterial plasmid has stably integrated based on the detectable expression of the first fluorescent marker gene, preferably by flow cytometry; and
(iv) isolating the identified eukaryotic host cell to obtain the eukaryotic host cell or cell line comprising the landing pad for λ-integrase-mediated recombination.

2. The method of claim 1, wherein the eukaryotic host cell is a higher eukaryotic host cell, preferably a mammalian host cell, more preferably a human cell, more preferably an embryonic kidney 293 (HEK 293) cell such as a human Expi293F cell.

3. The method of claim 1, wherein the eukaryotic host cell is a human cell, more preferably a human Expi293F cell,
wherein the RNA polymerase promoter is an EF-1α promoter;
wherein the modified lambda integrase recombination sequence attP comprises or consists of a nucleotide sequence according to SEQ ID NO:5 or a derivative thereof, and is downstream of the RNA polymerase promoter; and
wherein the first fluorescent marker gene is downstream of the modified lambda integrase recombination sequence attP sequence and is operably linked to the EF-1α promoter.

4. The method of claim 1, wherein the landing pad further comprises one or more additional recombination sequences such as loxP and/or FRT and/or attP.

5. The method of claim 1, wherein step (iv) comprises serial dilution or single cell FACS of the identified eukaryotic host cell to obtain a clonal eukaryotic host cell line comprising the landing pad for λ-integrase-mediated recombination, optionally further comprising generating a eukaryotic host cell line comprising the landing pad, preferably the eukaryotic host cell line is a monoclonal cell line, preferably the eukaryotic host cell line exhibits homogenous and stable long-term expression levels of the first fluorescent marker gene, preferably confirmed by flow cytometry analysis, in the absence of selection pressure.

6-7. (canceled)

8. The method of claim 1, further comprising, after step (iv), screening the isolated eukaryotic host cells for competency of λ-integrase-mediated recombination.

9. The method of claim 1, further comprising, after step (iv), confirming the eukaryotic host cell of step (iv) contains a single copy of the landing pad by Southern blotting analysis.

10. The method of claim 1, further comprising, after step (iv), confirming the integration of the landing pad into the genome of the eukaryotic host cell, by PCR analysis.

11. A eukaryotic host cell or cell line obtained from the method according to claim 1.

12. A method of λ-integrase-mediated insertion of a DNA sequence of interest into a eukaryotic host cell, the method comprising:
(i) providing a first circular DNA molecule comprising a lambda integrase recombination partner sequence of attP, and a DNA sequence of interest comprising a selection marker gene, preferably a hygromycin resistance gene, and a second fluorescent marker gene, preferably eGFP, operably linked to a constitutive promoter;
(ii) providing a second circular DNA molecule comprising a nucleotide sequence encoding a lambda integrase (IntC3) having the amino acid sequence set forth in SEQ ID NO:6 or a functional variant or fragment thereof,
(iii) co-transfecting a eukaryotic host cell according to claim 11 with the first circular DNA molecule of (i) and the second circular DNA molecule of (ii) at suitable conditions to induce said co-transfection; and
(iv) inducing the expression of the lambda integrase to facilitate recombination and stable integration of the first circular DNA construct into the landing pad comprised in the eukaryotic host cell;
(v) identifying a eukaryotic host cell comprising a landing pad into which the first circular DNA construct has stably integrated based on the detectable expression of the selection marker gene and second fluorescent marker gene, and the absence of the detectable expression of the first fluorescent marker gene.

13. The method of claim 12, wherein step (iv) results in the creation of two genomic recombination junction sequences flanking the DNA sequence of interest, optionally wherein the first fluorescent marker gene is downstream of a right genomic recombination junction sequence, and the RNA polymerase promoter is upstream of a left genomic recombination junction sequence.

14. (canceled)

15. The method of claim 12, wherein step (iv) comprises culturing the eukaryotic host cell in a selection medium under conditions that allow growth of the eukaryotic host cell expressing the selection marker gene and subsequently analysing the expression levels of the second fluorescent marker gene, and the first fluorescent marker gene, by using flow cytometry.

16. The method of claim 12, further comprising, after step (v), confirming the stable integration of the first circular DNA construct into the landing pad of the eukaryotic host cell, by PCR analysis, and/or digesting the genomic DNA of the eukaryotic host cell with one or more restriction enzymes and analysing the digested fragments by using Southern blot.

17. (canceled)

18. The method of claim 12, further comprising isolating the identified eukaryotic host cell comprising a landing pad into which the first circular DNA construct has stably integrated, by serial dilution.

19. The method of claim 12, wherein in the first circular DNA molecule, the selection marker gene is downstream of the lambda integrase recombination partner sequence of attP, and the second fluorescent marker gene is downstream of the selection marker gene.

20. The method of claim 12, wherein the second fluorescent marker gene is comprised in an expression cassette with the constitutive promoter suitable for controlling expression of the second fluorescent marker gene, preferably the constitutive promoter is a Chicken β-actin promoter, and optionally the expression cassette comprises a second selection marker gene, preferably a puromycin resistance gene.

21. The method of claim 12, wherein the second circular DNA molecule further comprises a nucleotide sequence encoding for an integration host factor, preferably single chain integration host factor 2 (scIHF2), optionally the lambda integrase and integration host factor are comprised in an expression cassette.

22. (canceled)

23. The method of claim 12, wherein the DNA sequence of interest comprises one or more additional genes, optionally the one or more additional genes comprises two genes, wherein the two genes are orientated head-to-tail (CW) or head-to-head (CCW) relative to each other, preferably the one or more additional genes comprise monoclonal antibody IgG PD-1 heavy and light chain genes, wherein the two IgG PD-1 genes are orientated head-to-tail (CW) or head-to-head (CCW) relative to each other.

24-25. (canceled)

26. The method of claim 12, wherein the expression of genes comprised in the DNA sequence of interest is stable and sustained for at least two weeks in the absence of selection pressure.

27. The method of claim 12, further comprising, after step (iv), encapsulating the eukaryotic host cell, preferably using a cellulose sulfate-based encapsulation protocol.

28. (canceled)


SEQUENCE LISTING (32 sequences)

SEQ ID NO: 1 - DNA, 243 bp, Enterobacteria phage lambda
  source 1..243: mol_type = genomic DNA
tctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttc

SEQ ID NO: 2 - DNA, 20 bp, Escherichia coli
  source 1..20: mol_type = genomic DNA
ctgcttttttatactaactt

SEQ ID NO: 3 - DNA, 103 bp, Escherichia coli
  source 1..103: mol_type = genomic DNA
ctgcttttttatactaagttggcattataaaaaagcattgcttatcaatttgttgcaacgaacaggtcactatcagtcaaaataaaaatcattatttgatttc

SEQ ID NO: 4 - DNA, 162 bp, Escherichia coli
  source 1..162: mol_type = genomic DNA
tctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttg

SEQ ID NO: 5 - DNA, 243 bp, Synthetic construct
  source 1..243: mol_type = other DNA
tctgttacaggtcactaataccatctaagtagttgattcatagtgactgcatatcttgtgttttacagtattaattagtctgttttttatccaaaatctaatttaatatattgatatttatatcattttacgtttctcgttcagcttttttatactaagttggcattataaaaaagcattgcttatcaatttgttgcaacgaacaggtcactatcagtcaaaatacaatcattatttgatttc

SEQ ID NO: 6 - protein, 355 aa, Synthetic construct
MGRRRSHERRDLPPNLYIRNNGYYCYRDPRTGKEFGLGRDRRFAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNHVAATRAAKSKVRRSRLTADEYLKIYQAAESSPCWLRLAMELAWTGQRVGDLCKMKWSDIVDGYLYVEQSKTGVKIAIPTALHIDALGISMKETLDKCKEILGGETIIASTRREPLSSGTVSRYFMRARKASGLSFEGDPPTFHELRSLSARLYGKQISDKFAQHLLGHKSVTMASQYRDDRGREWDKIEIK

SEQ ID NO: 7 - DNA, 24 bp, synthetic construct
  source 1..24: mol_type = other DNA
gtccaggcacctcgattagttctc

SEQ ID NO: 8 - DNA, 25 bp, synthetic construct
  source 1..25: mol_type = other DNA
cgaagctgaaagcacgagattcttc

SEQ ID NO: 9 - DNA, 24 bp, synthetic construct
  source 1..24: mol_type = other DNA
gataagtcgtgtcttaccgggttg

SEQ ID NO: 10 - DNA, 24 bp, synthetic construct
  source 1..24: mol_type = other DNA
cttgtagatgaactcgccgtcctg

SEQ ID NO: 11 - DNA, 22 bp, synthetic construct
  source 1..22: mol_type = other DNA
atggtgagcaagggcgaggagg

SEQ ID NO: 12 - DNA, 23 bp, synthetic construct
  source 1..23: mol_type = other DNA
gcttcaagtagtcggggatgtcg

SEQ ID NO: 13 - DNA, 21 bp, synthetic construct
  source 1..21: mol_type = other DNA
ccgaccacatgaagcagcacg

SEQ ID NO: 14 - DNA, 22 bp, synthetic construct
  source 1..22: mol_type = other DNA
gttctgctggtagtggtcggcg

SEQ ID NO: 15 - DNA, 27 bp, synthetic construct
  source 1..27: mol_type = other DNA
aatggctagagacttatcgaaagcagc

SEQ ID NO: 16 - DNA, 23 bp, synthetic construct
  source 1..23: mol_type = other DNA
cgacatccccgactacttgaagc

SEQ ID NO: 17 - DNA, 23 bp, synthetic construct
  source 1..23: mol_type = other DNA
cgacatccccgactacttgaagc

SEQ ID NO: 18 - DNA, 28 bp, synthetic construct
  source 1..28: mol_type = other DNA
tacacgacatcactttcccagtttaccc

SEQ ID NO: 19 - DNA, 27 bp, synthetic construct
  source 1..27: mol_type = other DNA
gtcaacatcaagttggacatcacctcc

SEQ ID NO: 20 - DNA, 26 bp, synthetic construct
  source 1..26: mol_type = other DNA
gtagtttgggtttgtttgtctccagc

SEQ ID NO: 21 - DNA, 24 bp, synthetic construct
  source 1..24: mol_type = other DNA
ctttctcatagctcacgctgtagg

SEQ ID NO: 22 - DNA, 27 bp, synthetic construct
  source 1..27: mol_type = other DNA
gaagtgaaagggaagacaagaacatgg

SEQ ID NO: 23 - DNA, 25 bp, synthetic construct
  source 1..25: mol_type = other DNA
cgtcgtttggtatggcttcattcag

SEQ ID NO: 24 - DNA, 25 bp, synthetic construct
  source 1..25: mol_type = other DNA
gaggaagagttcttgcagctcggtg

SEQ ID NO: 25 - DNA, 23 bp, synthetic construct
  source 1..23: mol_type = other DNA
ggaaccgctcaactcggccatgc

SEQ ID NO: 26 - DNA, 25 bp, synthetic construct
  source 1..25: mol_type = other DNA
tgacggtactgggacaacctaaagc

SEQ ID NO: 27 - DNA, 25 bp, synthetic construct
  source 1..25: mol_type = other DNA
gactacggggcaggtccttactact

SEQ ID NO: 28 - DNA, 21 bp, synthetic construct
  source 1..21: mol_type = other DNA
ccaaagcgtcctcactcaacc

SEQ ID NO: 29 - DNA, 23 bp, synthetic construct
  source 1..23: mol_type = other DNA
gtaacagacggagccgctttagg

SEQ ID NO: 30 - DNA, 33 bp, synthetic construct
  source 1..33: mol_type = other DNA
ctatggatccccaccatggtgagcaagggcgag

SEQ ID NO: 31 - DNA, 60 bp, synthetic construct
  source 1..60: mol_type = other DNA
tgccaagcttataacttcgtatagcatacattatacgaagttatcccgatccagacatga

SEQ ID NO: 32 - DNA, 1980 bp, Homo sapiens
  source 1..1980: mol_type = genomic DNA
gttgtgtctatgtctgaacagagtggtccaggaggcaaattcttagacctatcagggagaataagcagtggtcttgccttcaccagtaacccactgtttcccttacctttcaattggattaacgacataaagaaataaatgagtgtaaagaaatgagacaagactacataagagggagactccagtcgcattcatggttggggtctgtacattttcttgccgggatctgactgccaaggcgtgatccacacatacatgggtgttcattgtagtttgggtttgtttgtctccagcaaaaataggtaggtgcctaggtaccggctcgccctgatttctctcttcttaagtctttttgaggtcttcgtgaaattaacatcttataatagaatatcttacattccattgctgctgggagtcttgttgctgtgattgttttgtctccctaagcccctttgaggccagacctgtaactcacagttgcttcccttctgggtcctggcacagggtggggcccaggtgctagctaccctgtgcacctgtgcaatgttattgggttgtggttcagtgttttacagagctcatcaagcatggggaatgccccactgaccatctctaaatgggtctaagctgggaagcttgcagcagttcttgtatatctgtggcaagacagcttagtaagtttttgtgaagccccttgtcctcgctgccagcctccctgggagcttctgtggggacagtgagtagccctgaccgcagcctgagtggctcagtcatcactggggccacagcccaccttaaatgttgtcagggcctggcgaaggggcacatggagccttgtgccatagttcctgaggtttgatctcaccctgcccaccttagccaggcaatccctgcccaccaggtgccccccaccacctgcataggcagacatcaaactttttagattcctcccaagacctcgatgctggagagccagacactaattcagggcctttggaggccccagcagggcacgcactggtcctcagcaccagtgcactcagccttgccagaaagaagctgcatgcatgtagaccagcaggagtcgttgctgattgccgactgttttggtaactaaaatggactacctgtgttggctttaagcagtagttgtgaaattgtgtgtcaggaagagggcttcctccacgggtctaccaagtggtctaccacttgctgagtgcagagccttgtcctggtctggatgcgacgatggaaggcttaagtgagtcatctcctgaagagactactcaggactggaagtgtgtcagcctaggggcagctgaacacccaggtggccctggagcaggctgctggagcgtctggcctgcagccggtggaagtaactgagctccaatcttgtgtccagactgcttctcccctaggctccagctgtttttctgcttctcctgtcactcatatggacagtgcactttccactgagcaggtcaatggcggggttacttttacacctaactttagctcctctctgggccctgctggtctcagcacgcacacccacccaatcctacggagacagagttctcacacctaccctccgcagggcatcttgggccatccacgtccacctgccctcccttcagcgtggccatcacggtgccctgactcagcttccagtcccccgggcttccatgttcttgtcttccctttcacttctacctttcctgcttgagtaagttaggctgtggctcgcaaacttcagtgagcatttgagtgttgtcagaaggcccccaggatggctaaatagtagaaaggacagctttactggtgacatcagtttgcaagccaggaatagatagtctctgacatggaacgaaggtgctctcgcttcgcagaaggggaagggcagttggattttatggctcgcagggtctgtgttacatagtagaatcagacatgtttagcaggtttgggggaaagctg